(19) United States
(12) Patent Application Publication
(10) Pub. No.: US 2024/0376091 A1
(43) Pub. Date: Nov. 14, 2024

(54) THIADIAZOLEAMIDE DERIVATIVES AND THEIR APPLICATIONS

(71) Applicant: ZHEJIANG UNIVERSITY, HANGZHOU, Zhejiang Province (CN)

(72) Inventors: TINGJUN HOU, HANGZHOU, ZHEJIANG PROVINCE (CN); DAN LI, HANGZHOU, ZHEJIANG PROVINCE (CN); RONG SHENG, HANGZHOU, ZHEJIANG PROVINCE (CN); WEITAO FU, HANGZHOU, ZHEJIANG PROVINCE (CN); CHENXIAN HU, HANGZHOU, ZHEJIANG PROVINCE (CN); HAO YANG, HANGZHOU, ZHEJIANG PROVINCE (CN); MINKUI ZHANG, HANGZHOU, ZHEJIANG PROVINCE (CN); JIANING LIAO, HANGZHOU, ZHEJIANG PROVINCE (CN)

(21) Appl. No.: 18/292,906

(22) PCT Filed: Aug. 19, 2021
(86) PCT No.: PCT/CN2021/113459

§ 371 Date: Jan. 27, 2024

(30) Foreign Application Priority Data
Jul. 30, 2021 (CN) 202110870155.0

(51) Int. Cl. C07D 417/12; A61K 31/433; A61K 31/4439; A61K 31/454; A61K 31/496; A61K 31/5377; A61P 35/00; C07D 417/14

(52) U.S. Cl. CPC C07D 417/12; A61K 31/433; A61K 31/4439; A61K 31/454; A61K 31/496; A61K 31/5377; A61P 35/00; C07D 417/14

(57) ABSTRACT

This invention pertains to compounds of the general formula (I) and their applications. The invention introduces a novel class of compounds exhibiting androgen receptor (AR) antagonistic activity. In particular, these compounds target a binding pocket located in the dimer interface of AR ligand binding domain (AR-LBD). These compounds effectively disrupt AR dimerization, thereby suppressing AR signaling. The high AR antagonistic activity and favorable safety profile of the compounds provided by the present invention have been substantiated through biochemical assays and animal models. Therefore, these compounds can be utilized in the formulation of medications to treat diseases characterized by abnormal expression of AR, including but not limited to prostate cancer, metastatic prostate cancer, castration-resistant prostate cancer, breast cancer and ovarian cancer.

### FIELD OF INVENTION

The present invention pertains to the field of medicine, specifically to the medicinal use of thiadiazolamide compounds, and their application in drug preparation for treating prostate cancer.

### BACKGROUND TECHNOLOGY

Globally, prostate cancer (PCa) has been seriously threatening the health of men, and continues to be the second most common cancer and also the fifth leading cause of cancer death. PCa patients are generally asymptomatic in the early stages, and the diagnosis often occurs when the cancer is already in the middle or advanced stages. Normal prostate cells and prostate cancer cells are both dependent on androgen for growth. Surgical or drug castration can greatly reduce the level of androgen for PCa patients in the early stages, thereby inhibiting androgen-androgen receptor (AR)-dependent signaling pathways and further inhibiting the growth of PCa cells. However, as the course of PCa progresses, most of the PCa patients eventually develop castration-resistant PCa.

To date, targeted small molecule therapeutics for PCa mainly fall into two categories: those targeting P450 CYP17A1 (Abiraterone) or AR (Bicalutamide, Enzalutamide, etc.). For AR targeting drugs, disrupting the interaction between AR and androgen is one of the crucial approaches in treating advanced and metastatic PCa.

AR is a member of the nuclear receptor (NR) family, and it contains 920 amino acids with four functional modules: a N-terminal domain (NTD), a DNA binding domain (DBD), a hinge region, and a C-terminal ligand binding domain (LBD). Androgens bind to the ligand binding pocket (LBP) of the AR-LBD. The unactivated AR is normally located in the cytoplasm and forms a stable complex with heat shock proteins. When androgens binding. AR dissociates from the heat shock protein and translocates into the cell nucleus, and acts as a transcriptional factor as homodimer. During the process, the recruitment of coactivators to the activation function-2 (AF-2) of the AR-LBD, binding to the androgen response element (ARE) in the DBD domain, and the recruitment of a series of other cofactors are also important. Finally, the activation or repression of specific gene transcription can regulate the growth, proliferation and metastasis of PCa cells.

At present, except for the flexible hinge region, many small-molecule antagonists targeting the domains of AR-NTD. AR-DBD, and AR-LBD have been reported. However, all the approved drugs target the LBP of the AR-LBD, including the first-generation AR antagonists (Flutamide, Nilutamide, R-bicalutamide) and the second-generation AR antagonists (Enzalutamide (Enz), Apalutamide, and Darolutamide). Especially, the second-generation antagonists have demonstrated encouraging therapeutic effects in clinical practice. Nevertheless, the emergence of intrinsic or acquired resistance would eventually reduce the clinical benefits of drugs, including both first- and second-generation AR antagonists.

To date, most drug resistance mutations have emerged in the LBP of AR-LBD, including F876L, T878A, H875Y, and W742C, etc. These resistance mutations directly affect the binding of drugs to the AR LBP, resulting in less effective. Meanwhile, the emergence of drug resistance mutations often leads to multi-drug resistance, severely limiting clinical benefits.

The design of novel AR antagonists with new scaffolds or new molecules bound to a novel binding site is still urgently required. This could contribute to overcoming drug resistance and greatly benefit patients by providing more selections of treatment plans.

### SUMMARY OF THE INVENTION

The object of the present invention is to provide a novel class of compounds with AR antagonistic activity for the preparation of drugs and the treatment of AR-related diseases (PCa, etc.).

To achieve the above objectives, the present invention adopts the following technical scheme:

A thiadiazolamide compound of Formula (I):

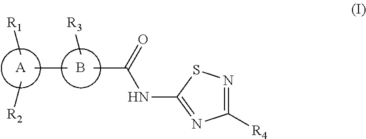

wherein:
ring A is selected from

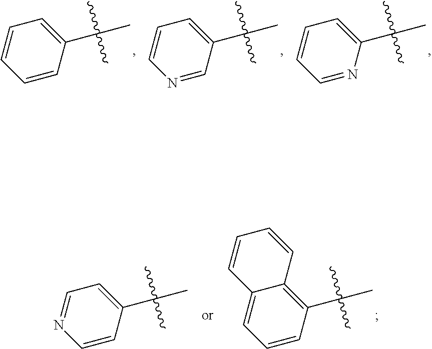 [SMILES: *c1ccccc1 ; *c1cccnc1 ; *c1ccccn1 ; *c1ccncc1 ; *c1cccc2ccccc12]

R₁ and R₂ are each independently selected from hydrogen, halogen, —NO₂, —CN, —OH, —NH₂, —SCH₃, carboxyl, ester, C₁₋₆ alkyl, C₁₋₆ fluoroalkyl, C₁₋₆ alkoxy, C₁₋₆ fluoroalkoxy, C₁₋₆ alkylamine group, C₁₋₆ dialkylamine group, C₃₋₆ cycloalkyl amine group, C₁₋₃ alkyl sulfonyl, C₁₋₃ fluoroalkyl sulfonyl, —NR₅R₆, CH₃ (CH₂), CONH— (n=1-6), —CONH₂, —CONR₅R₆, —SO₂NH₂, —SO₂NR₅R₆;
ring B is selected from

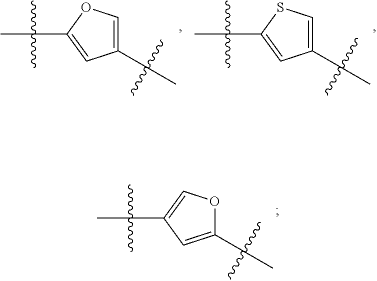 [SMILES: *c1coc(*)c1 ; *c1csc(*)c1 ; *c1coc(*)c1]

R₃ is selected from hydrogen, halogen, —NO₂, —CN, —OH, —NH₂, —SO₂NH₂, C₁₋₆ alkyl, C₁₋₆ fluoroalkyl, C₁₋₆ alkoxy, C₁₋₆ fluoroalkoxy;
R₄ is selected from hydrogen, halogen, C₁₋₆ alkyl, C₁₋₆ fluoroalkyl, C₁₋₆ alkoxy, C₁₋₆ fluoroalkoxy, C₁₋₆ alkylamine group, C₁₋₆ dialkylamine group, C₃₋₆ cycloalkyl amine group, CH₃ (CH₂), CONH— (n=1-6), —CONH₂, —CONR₅R₆, —CH₂COCH₃, —CH₂NR₅R₆, —CH₂CF₂CH₃, —CH₂C(═CF₂) CH₃, —CH₂C(═NHOH) CH₃, —CH₂C(═NHOCH₃)CH₃, —CH₂R₇, —CH₂C(R₇R₈)CH₃;
R₅ and R₆ are each independently selected from hydrogen, C₁₋₄ alkyl, C₁₋₄ cycloalkyl, or NR₅R₆ is 3-8 optionally substituted cyclic amine selected from morpholine, piperazine, methylpiperazine, pyrrolidine, piperidine, the substituent group is selected from hydrogen, C₁₋₆ alkyl, C₁₋₆ alkoxy, C₂₋₆ unsaturated aliphatic hydrocarbon group, C₃₋₈ cycloalkyl, C₃₋₈ unsaturated alicyclic group, C₃₋₈ saturated heterocyclic group, halogen, —NH₂, —CN;
R₇ and R₈ are each independently selected from hydrogen, fluoro, —OH, —NH₂, —CN, C₁₋₆ alkyl, C₁₋₆ fluoroalkyl, C₁₋₆ alkoxy, C₁₋₆ fluoroalkoxy, C₁₋₆ alkylamine group, C₁₋₆ dialkylamine group, optionally substituted C₃₋₈ cyclic amine selected from morpholine, piperazine, methylpiperazine, pyrrolidine, piperidine, the substituent group is selected from hydrogen, C₁₋₆ alkyl, C₁₋₆ alkoxy, C₂₋₆ unsaturated aliphatic hydrocarbon group, C₃₋₈ cycloalkyl, C₃₋₈ unsaturated alicyclic group, C₃₋₈ saturated heterocyclic group, halogen, —NH₂, —CN.

Preferably, the R₁ and R₂ of compounds are each independently selected from hydrogen, —F, —Cl, —NO₂, —CN, —CH₃, —OCH₃, —OCF₂H, —OCF₃, difluoroethoxy, trifluoroethoxy, CH₃SO₂—, CF₃SO₂—, —N(CH₃)₂, —N(CH₂CH₃)₂, CH₃CONH—, —CONH₂, —CONHCH₃, —SO₂NH₂, —SO₂NHCH₃.
R₃ is selected from hydrogen, —F, —Cl, —NO₂, —CH₃, —CH₂CH₃, —CF₃, —OCH₃, —OCF₂H, —OCF₃.
R₄ is selected from hydrogen, —F, —Cl, —CH₃, —N(CH₃)₂, —CONH₂, —CONH₂CH₂CH₃,

More preferably, the chemical structures are illustrated in table 1.

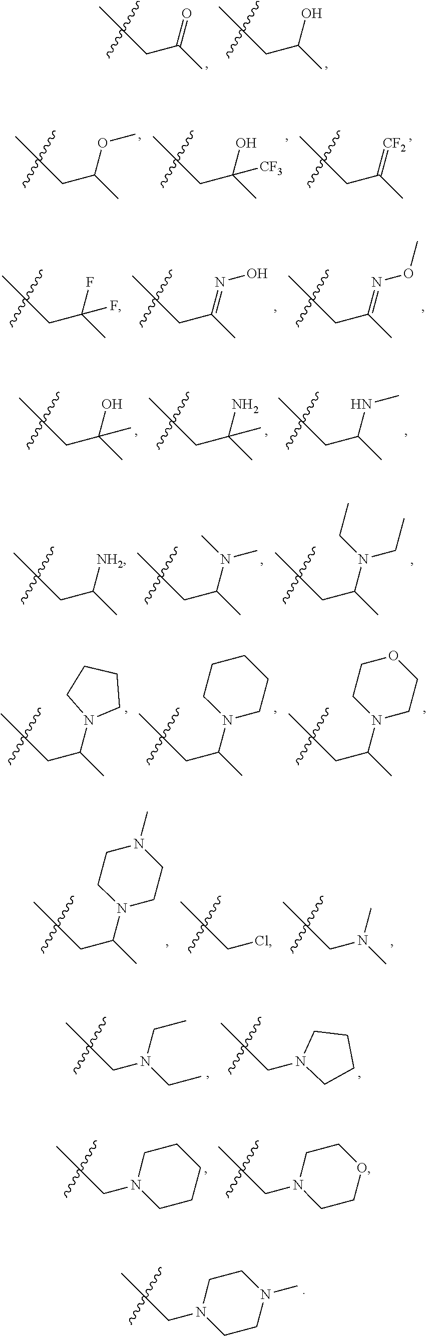

More preferably, the chemical structures are illustrated in table 1.

TABLE 1

| NO. | Structure |
|---|---|
| A-1 | 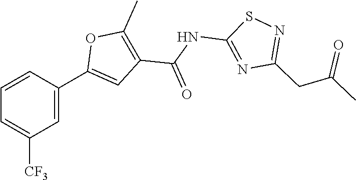 [SMILES: CC(=O)Cc1nsc(NC(=O)c2cc(-c3cccc(C(F)(F)F)c3)oc2C)n1] |
| A-2 | 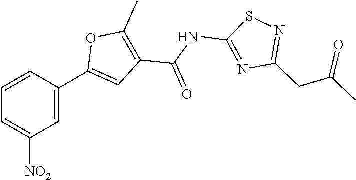 [SMILES: CC(=O)Cc1nsc(NC(=O)c2cc(-c3cccc([N+](=O)[O-])c3)oc2C)n1] |
| A-3 | 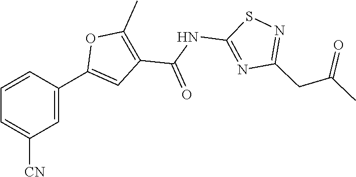 [SMILES: CC(=O)Cc1nsc(NC(=O)c2cc(-c3cccc(C#N)c3)oc2C)n1] |
| A-4 | 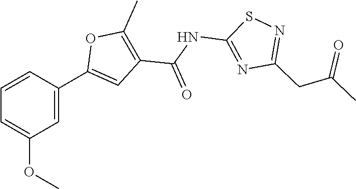 [SMILES: COc1cccc(-c2cc(C(=O)Nc3nc(CC(C)=O)ns3)c(C)o2)c1] |
| A-5 | 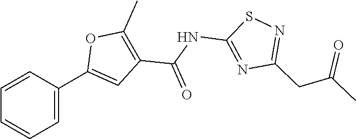 [SMILES: CC(=O)Cc1nsc(NC(=O)c2cc(-c3ccccc3)oc2C)n1] |
| A-6 | 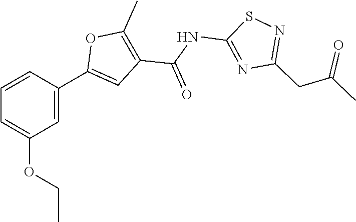 [SMILES: CCOc1cccc(-c2cc(C(=O)Nc3nc(CC(C)=O)ns3)c(C)o2)c1] |
| A-7 | 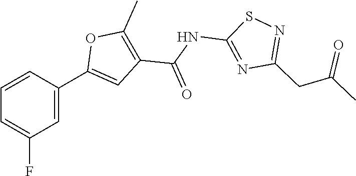 [SMILES: CC(=O)Cc1nsc(NC(=O)c2cc(-c3cccc(F)c3)oc2C)n1] |
| A-8 | 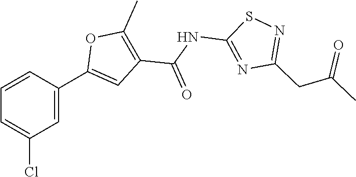 [SMILES: CC(=O)Cc1nsc(NC(=O)c2cc(-c3cccc(Cl)c3)oc2C)n1] |
| A-9 | 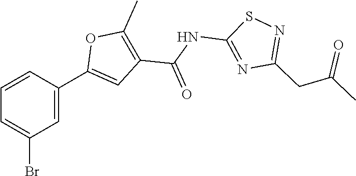 [SMILES: CC(=O)Cc1nsc(NC(=O)c2cc(-c3cccc(Br)c3)oc2C)n1] |
| A-10 | 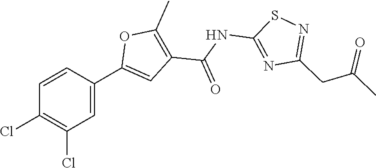 [SMILES: CC(=O)Cc1nsc(NC(=O)c2cc(-c3ccc(Cl)c(Cl)c3)oc2C)n1] |
| A-11 | 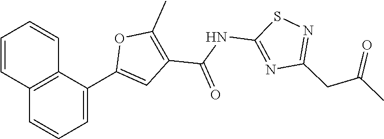 [SMILES: CC(=O)Cc1nsc(NC(=O)c2cc(-c3cccc4ccccc34)oc2C)n1] |
| A-12 | 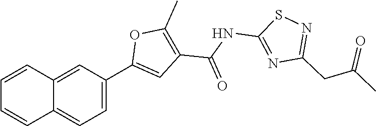 [SMILES: CC(=O)Cc1nsc(NC(=O)c2cc(-c3ccc4ccccc4c3)oc2C)n1] |
| A-13 | 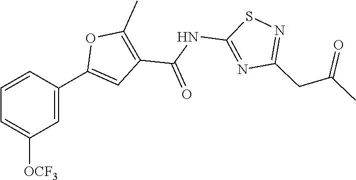 [SMILES: CC(=O)Cc1nsc(NC(=O)c2cc(-c3cccc(OC(F)(F)F)c3)oc2C)n1] |
| A-14 | 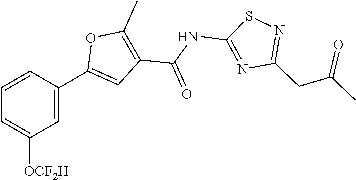 |
| A-24 | 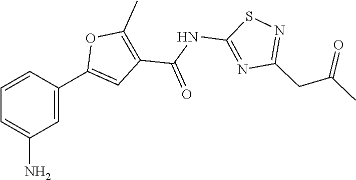 [SMILES: CC(=O)Cc1nsc(NC(=O)c2cc(-c3cccc(N)c3)oc2C)n1] |
| A-25 | 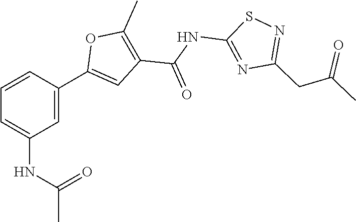 [SMILES: CC(=O)Cc1nsc(NC(=O)c2cc(-c3cccc(NC(C)=O)c3)oc2C)n1] |
| A-26 | 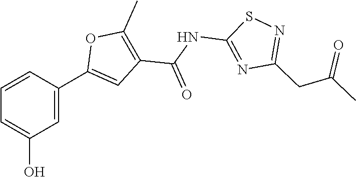 [SMILES: CC(=O)Cc1nsc(NC(=O)c2cc(-c3cccc(O)c3)oc2C)n1] |
| A-27 | 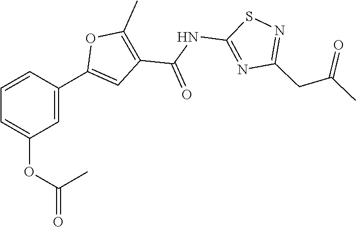 [SMILES: CC(=O)Cc1nsc(NC(=O)c2cc(-c3cccc(OC(C)=O)c3)oc2C)n1] |
| A-28 | 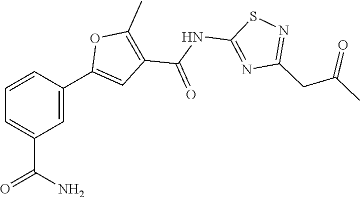 [SMILES: CC(=O)Cc1nsc(NC(=O)c2cc(-c3cccc(C(N)=O)c3)oc2C)n1] |
| A-29 | 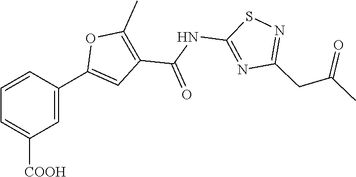 [SMILES: CC(=O)Cc1nsc(NC(=O)c2cc(-c3cccc(C(=O)O)c3)oc2C)n1] |
| A-30 | 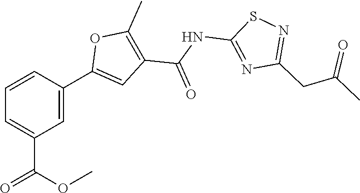 [SMILES: COC(=O)c1cccc(-c2cc(C(=O)Nc3nc(CC(C)=O)ns3)c(C)o2)c1] |
| A-31 | 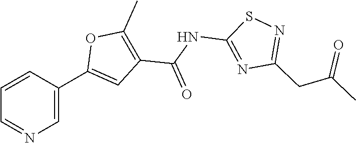 [SMILES: CC(=O)Cc1nsc(NC(=O)c2cc(-c3cccnc3)oc2C)n1] |
| A-34 | 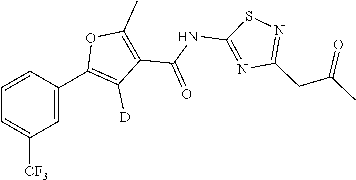 [SMILES: [2H]c1c(-c2cccc(C(F)(F)F)c2)oc(C)c1C(=O)Nc1nc(CC(C)=O)ns1] |
| A-35 | 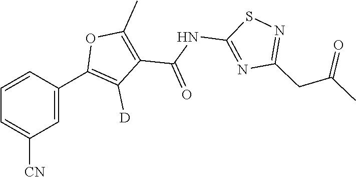 [SMILES: [2H]c1c(-c2cccc(C#N)c2)oc(C)c1C(=O)Nc1nc(CC(C)=O)ns1] |
| A-36 | 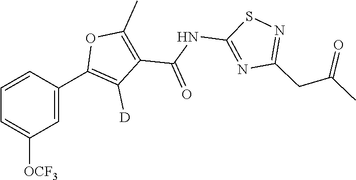 [SMILES: [2H]c1c(-c2cccc(OC(F)(F)F)c2)oc(C)c1C(=O)Nc1nc(CC(C)=O)ns1] |
| A-37 | 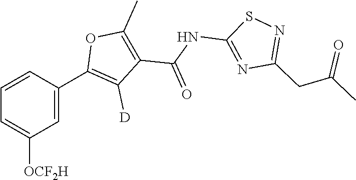 |
| A-38 | 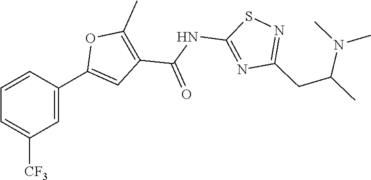 [SMILES: Cc1oc(-c2cccc(C(F)(F)F)c2)cc1C(=O)Nc1nc(CC(C)N(C)C)ns1] |
| A-39 | 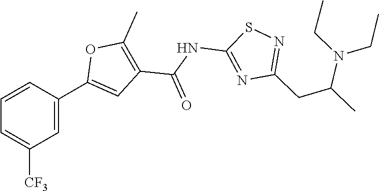 [SMILES: CCN(CC)C(C)Cc1nsc(NC(=O)c2cc(-c3cccc(C(F)(F)F)c3)oc2C)n1] |
| A-40 | 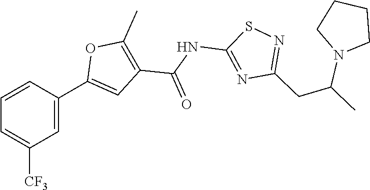 [SMILES: Cc1oc(-c2cccc(C(F)(F)F)c2)cc1C(=O)Nc1nc(CC(C)N2CCCC2)ns1] |
| A-41 | 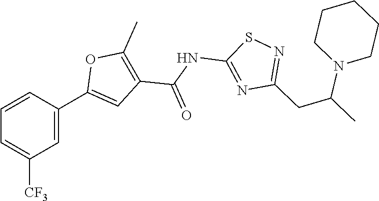 [SMILES: Cc1oc(-c2cccc(C(F)(F)F)c2)cc1C(=O)Nc1nc(CC(C)N2CCCCC2)ns1] |
| A-42 | 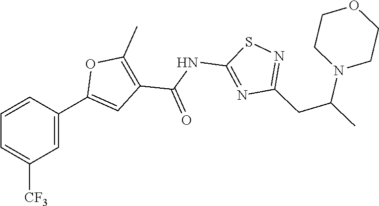 [SMILES: Cc1oc(-c2cccc(C(F)(F)F)c2)cc1C(=O)Nc1nc(CC(C)N2CCOCC2)ns1] |
| A-43 | 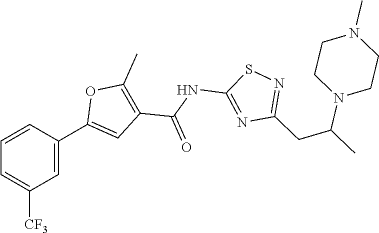 [SMILES: Cc1oc(-c2cccc(C(F)(F)F)c2)cc1C(=O)Nc1nc(CC(C)N2CCN(C)CC2)ns1] |
| A-44 | 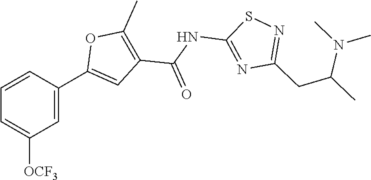 [SMILES: Cc1oc(-c2cccc(OC(F)(F)F)c2)cc1C(=O)Nc1nc(CC(C)N(C)C)ns1] |
| A-45 | 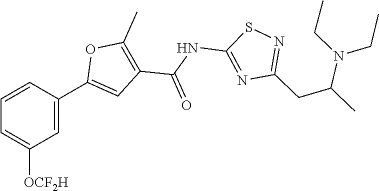 |
| A-46 | 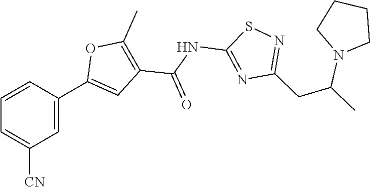 [SMILES: Cc1oc(-c2cccc(C#N)c2)cc1C(=O)Nc1nc(CC(C)N2CCCC2)ns1] |
| A-49 | 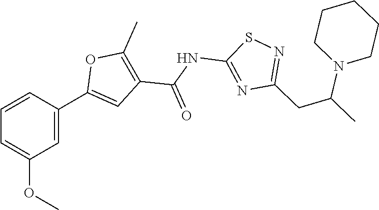 [SMILES: COc1cccc(-c2cc(C(=O)Nc3nc(CC(C)N4CCCCC4)ns3)c(C)o2)c1] |
| A-52 | 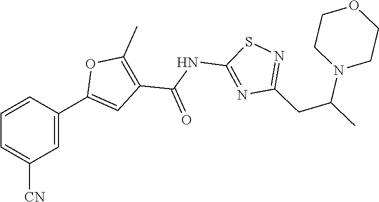 [SMILES: Cc1oc(-c2cccc(C#N)c2)cc1C(=O)Nc1nc(CC(C)N2CCOCC2)ns1] |
| A-56 | 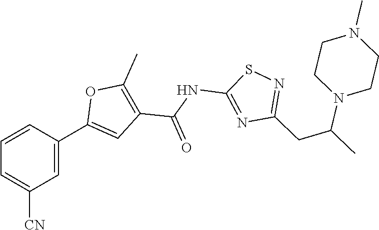 [SMILES: Cc1oc(-c2cccc(C#N)c2)cc1C(=O)Nc1nc(CC(C)N2CCN(C)CC2)ns1] |
| A-60 | 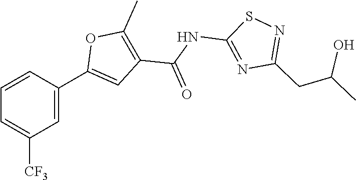 [SMILES: Cc1oc(-c2cccc(C(F)(F)F)c2)cc1C(=O)Nc1nc(CC(C)O)ns1] |
| A-61 | 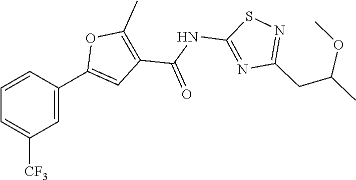 [SMILES: COC(C)Cc1nsc(NC(=O)c2cc(-c3cccc(C(F)(F)F)c3)oc2C)n1] |
| A-66 | 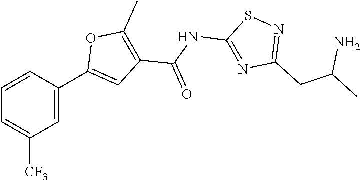 [SMILES: Cc1oc(-c2cccc(C(F)(F)F)c2)cc1C(=O)Nc1nc(CC(C)N)ns1] |
| A-69 | 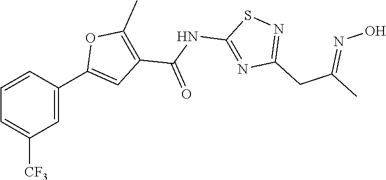 [SMILES: C/C(Cc1nsc(NC(=O)c2cc(-c3cccc(C(F)(F)F)c3)oc2C)n1)=N\O] |
| A-70 | 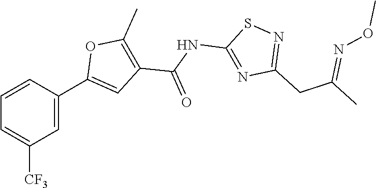 [SMILES: CO/N=C(\C)Cc1nsc(NC(=O)c2cc(-c3cccc(C(F)(F)F)c3)oc2C)n1] |
| A-75 | 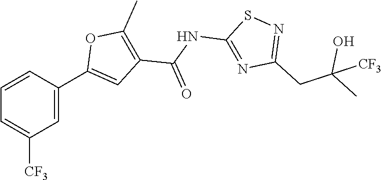 [SMILES: Cc1oc(-c2cccc(C(F)(F)F)c2)cc1C(=O)Nc1nc(CC(C)(O)C(F)(F)F)ns1] |
| A-80 | 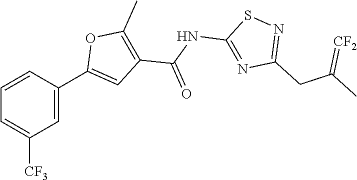 |
| A-85 | 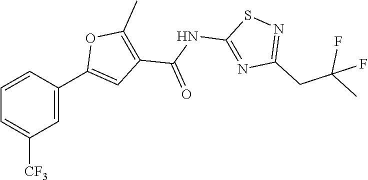 [SMILES: Cc1oc(-c2cccc(C(F)(F)F)c2)cc1C(=O)Nc1nc(CC(C)(F)F)ns1] |
| A-90 | 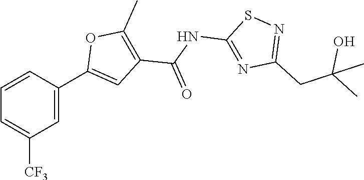 [SMILES: Cc1oc(-c2cccc(C(F)(F)F)c2)cc1C(=O)Nc1nc(CC(C)(C)O)ns1] |
| A-93 | 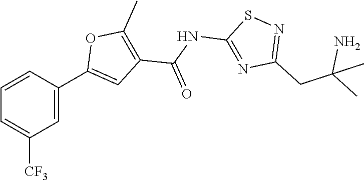 [SMILES: Cc1oc(-c2cccc(C(F)(F)F)c2)cc1C(=O)Nc1nc(CC(C)(C)N)ns1] |
| A-96 | 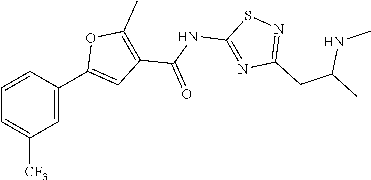 [SMILES: CNC(C)Cc1nsc(NC(=O)c2cc(-c3cccc(C(F)(F)F)c3)oc2C)n1] |
| A-99 | 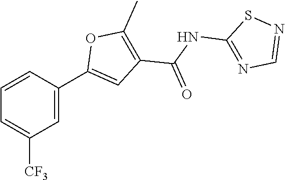 [SMILES: Cc1oc(-c2cccc(C(F)(F)F)c2)cc1C(=O)Nc1ncns1] |
| A-100 | 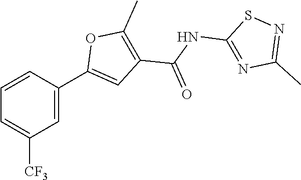 [SMILES: Cc1nsc(NC(=O)c2cc(-c3cccc(C(F)(F)F)c3)oc2C)n1] |
| A-105 | 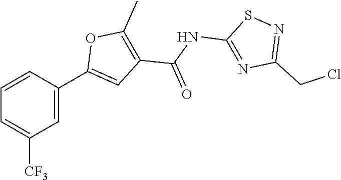 [SMILES: Cc1oc(-c2cccc(C(F)(F)F)c2)cc1C(=O)Nc1nc(CCl)ns1] |
| A-106 | 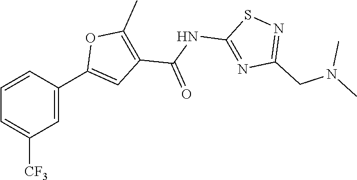 [SMILES: Cc1oc(-c2cccc(C(F)(F)F)c2)cc1C(=O)Nc1nc(CN(C)C)ns1] |
| A-107 | 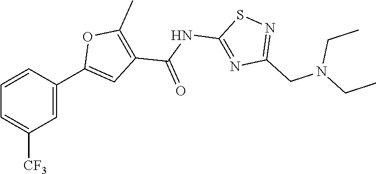 [SMILES: CCN(CC)Cc1nsc(NC(=O)c2cc(-c3cccc(C(F)(F)F)c3)oc2C)n1] |
| A-108 | 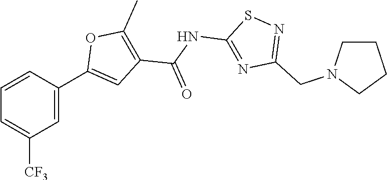 [SMILES: Cc1oc(-c2cccc(C(F)(F)F)c2)cc1C(=O)Nc1nc(CN2CCCC2)ns1] |
| A-109 | 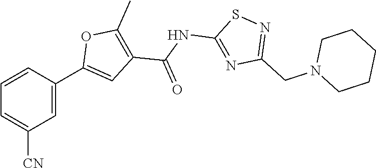 [SMILES: Cc1oc(-c2cccc(C#N)c2)cc1C(=O)Nc1nc(CN2CCCCC2)ns1] |
| A-110 | 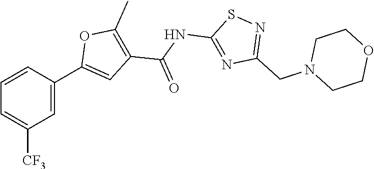 [SMILES: Cc1oc(-c2cccc(C(F)(F)F)c2)cc1C(=O)Nc1nc(CN2CCOCC2)ns1] |
| A-111 | 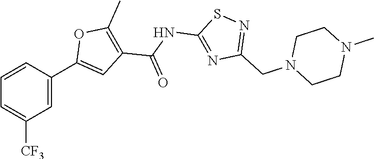 [SMILES: Cc1oc(-c2cccc(C(F)(F)F)c2)cc1C(=O)Nc1nc(CN2CCN(C)CC2)ns1] |
| A-112 | 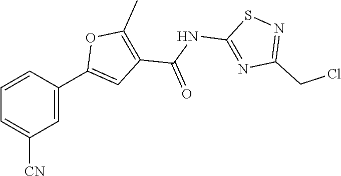 [SMILES: Cc1oc(-c2cccc(C#N)c2)cc1C(=O)Nc1nc(CCl)ns1] |
| A-118 | 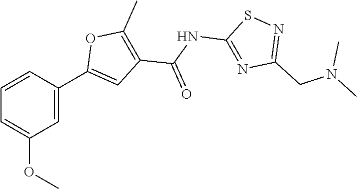 [SMILES: COc1cccc(-c2cc(C(=O)Nc3nc(CN(C)C)ns3)c(C)o2)c1] |
| A-123 | 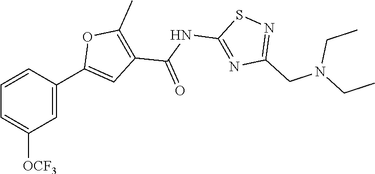 [SMILES: CCN(CC)Cc1nsc(NC(=O)c2cc(-c3cccc(OC(F)(F)F)c3)oc2C)n1] |
| A-124 | 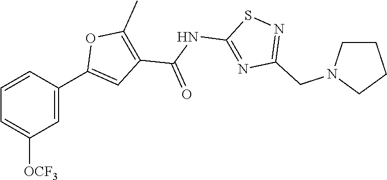 [SMILES: Cc1oc(-c2cccc(OC(F)(F)F)c2)cc1C(=O)Nc1nc(CN2CCCC2)ns1] |
| A-129 | 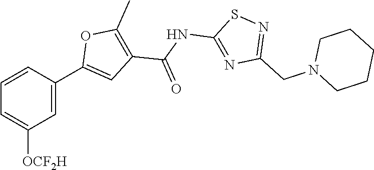 |
| A-130 | 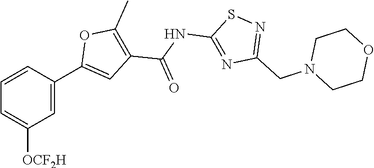 |
| A-131 | 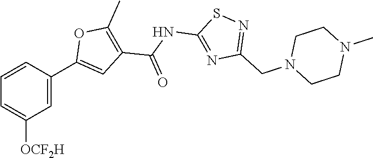 |
| B-1 | 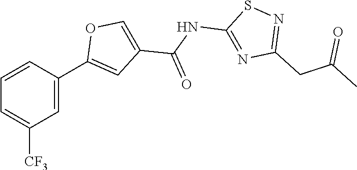 [SMILES: CC(=O)Cc1nsc(NC(=O)c2coc(-c3cccc(C(F)(F)F)c3)c2)n1] |
| B-2 | 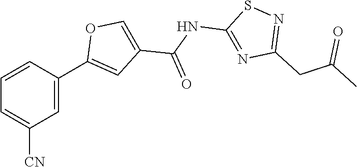 [SMILES: CC(=O)Cc1nsc(NC(=O)c2coc(-c3cccc(C#N)c3)c2)n1] |
| B-3 | 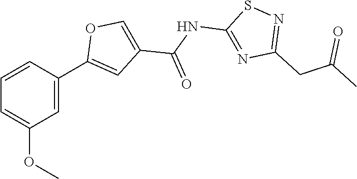 [SMILES: COc1cccc(-c2cc(C(=O)Nc3nc(CC(C)=O)ns3)co2)c1] |
| B-4 | 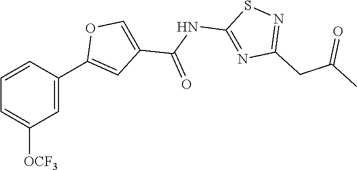 [SMILES: CC(=O)Cc1nsc(NC(=O)c2coc(-c3cccc(OC(F)(F)F)c3)c2)n1] |
| B-5 | 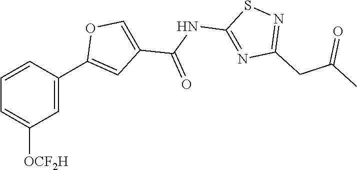 |
| B-6 | 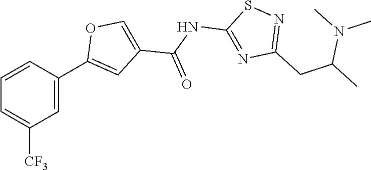 [SMILES: CC(Cc1nsc(NC(=O)c2coc(-c3cccc(C(F)(F)F)c3)c2)n1)N(C)C] |
| B-7 | 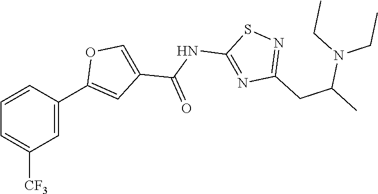 [SMILES: CCN(CC)C(C)Cc1nsc(NC(=O)c2coc(-c3cccc(C(F)(F)F)c3)c2)n1] |
| B-12 | 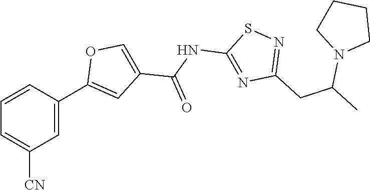 [SMILES: CC(Cc1nsc(NC(=O)c2coc(-c3cccc(C#N)c3)c2)n1)N1CCCC1] |
| B-14 | 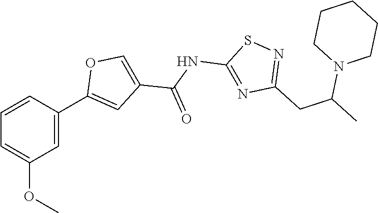 [SMILES: COc1cccc(-c2cc(C(=O)Nc3nc(CC(C)N4CCCCC4)ns3)co2)c1] |
| B-16 | 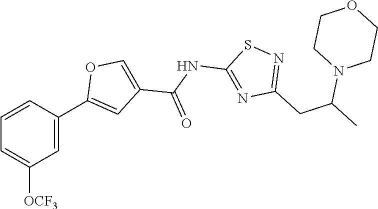 [SMILES: CC(Cc1nsc(NC(=O)c2coc(-c3cccc(OC(F)(F)F)c3)c2)n1)N1CCOCC1] |
| B-19 | 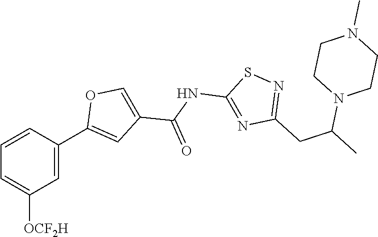 |
| B-20 | 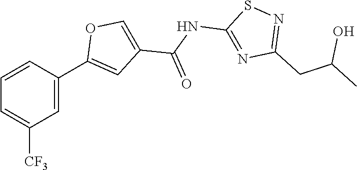 [SMILES: CC(O)Cc1nsc(NC(=O)c2coc(-c3cccc(C(F)(F)F)c3)c2)n1] |
| B-25 | 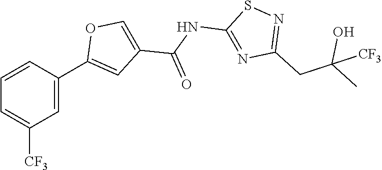 [SMILES: CC(O)(Cc1nsc(NC(=O)c2coc(-c3cccc(C(F)(F)F)c3)c2)n1)C(F)(F)F] |
| B-30 | 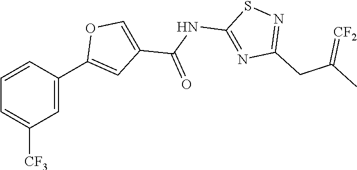 |
| B-35 | 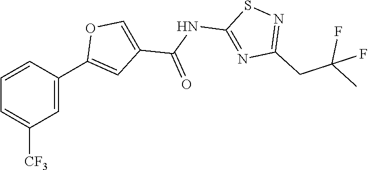 [SMILES: CC(F)(F)Cc1nsc(NC(=O)c2coc(-c3cccc(C(F)(F)F)c3)c2)n1] |
| B-40 | 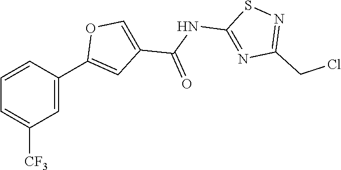 [SMILES: O=C(Nc1nc(CCl)ns1)c1coc(-c2cccc(C(F)(F)F)c2)c1] |
| B-47 | 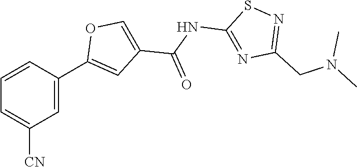 [SMILES: CN(C)Cc1nsc(NC(=O)c2coc(-c3cccc(C#N)c3)c2)n1] |
| B-50 | 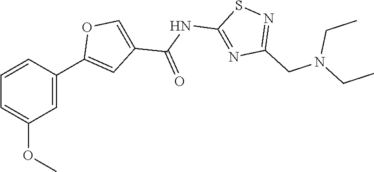 [SMILES: CCN(CC)Cc1nsc(NC(=O)c2coc(-c3cccc(OC)c3)c2)n1] |
| B-52 | 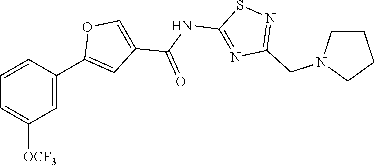 [SMILES: O=C(Nc1nc(CN2CCCC2)ns1)c1coc(-c2cccc(OC(F)(F)F)c2)c1] |
| B-56 | 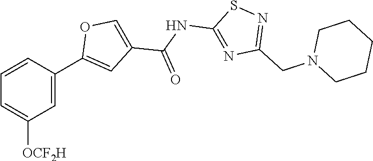 |
| B-57 | 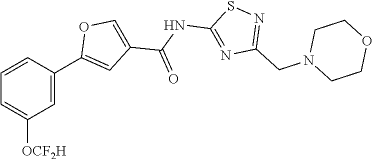 |
| B-58 | 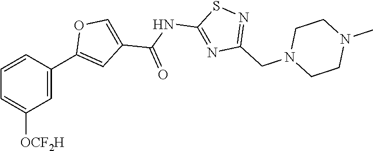 |
| C-1 | 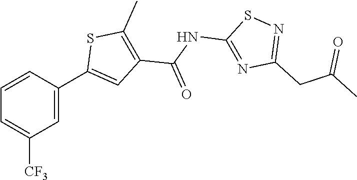 [SMILES: CC(=O)Cc1nsc(NC(=O)c2cc(-c3cccc(C(F)(F)F)c3)sc2C)n1] |
| C-2 | 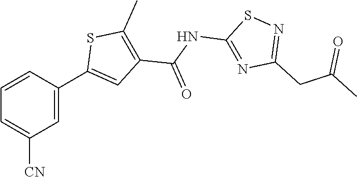 [SMILES: CC(=O)Cc1nsc(NC(=O)c2cc(-c3cccc(C#N)c3)sc2C)n1] |
| C-3 | 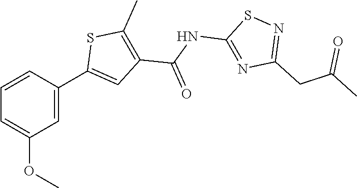 [SMILES: COc1cccc(-c2cc(C(=O)Nc3nc(CC(C)=O)ns3)c(C)s2)c1] |
| C-4 | 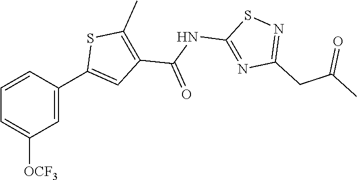 [SMILES: CC(=O)Cc1nsc(NC(=O)c2cc(-c3cccc(OC(F)(F)F)c3)sc2C)n1] |
| C-5 | 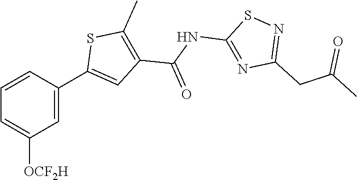 |
| C-6 | 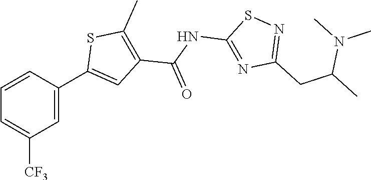 [SMILES: Cc1sc(-c2cccc(C(F)(F)F)c2)cc1C(=O)Nc1nc(CC(C)N(C)C)ns1] |
| C-7 | 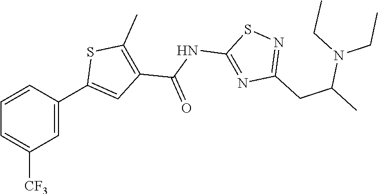 [SMILES: CCN(CC)C(C)Cc1nsc(NC(=O)c2cc(-c3cccc(C(F)(F)F)c3)sc2C)n1] |
| C-12 | 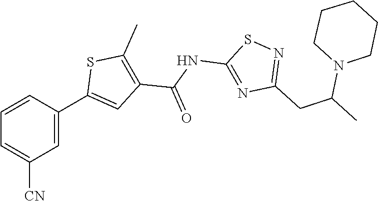 [SMILES: Cc1sc(-c2cccc(C#N)c2)cc1C(=O)Nc1nc(CC(C)N2CCCCC2)ns1] |
| C-14 | 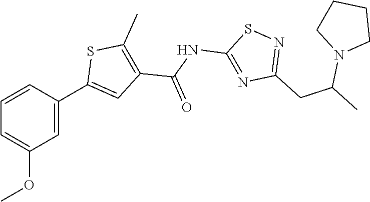 [SMILES: COc1cccc(-c2cc(C(=O)Nc3nc(CC(C)N4CCCC4)ns3)c(C)s2)c1] |
| C-17 | 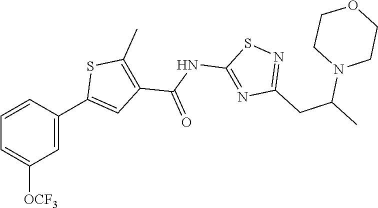 [SMILES: Cc1sc(-c2cccc(OC(F)(F)F)c2)cc1C(=O)Nc1nc(CC(C)N2CCOCC2)ns1] |
| C-19 | 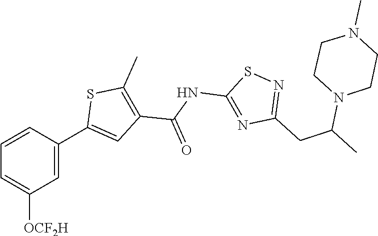 |
| C-20 | 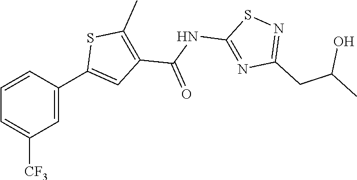 [SMILES: Cc1sc(-c2cccc(C(F)(F)F)c2)cc1C(=O)Nc1nc(CC(C)O)ns1] |
| C-25 | 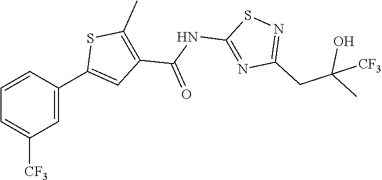 [SMILES: Cc1sc(-c2cccc(C(F)(F)F)c2)cc1C(=O)Nc1nc(CC(C)(O)C(F)(F)F)ns1] |
| C-30 | 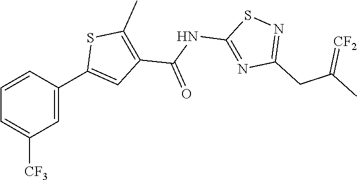 |
| C-35 | 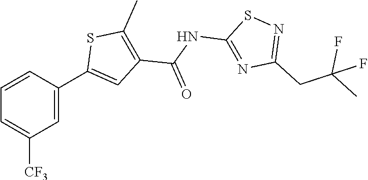 [SMILES: Cc1sc(-c2cccc(C(F)(F)F)c2)cc1C(=O)Nc1nc(CC(C)(F)F)ns1] |
| C-40 | 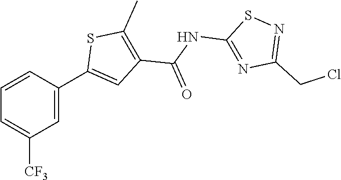 [SMILES: Cc1sc(-c2cccc(C(F)(F)F)c2)cc1C(=O)Nc1nc(CCl)ns1] |
| C-47 | 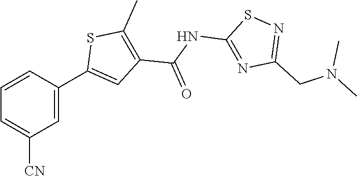 [SMILES: Cc1sc(-c2cccc(C#N)c2)cc1C(=O)Nc1nc(CN(C)C)ns1] |
| C-50 | 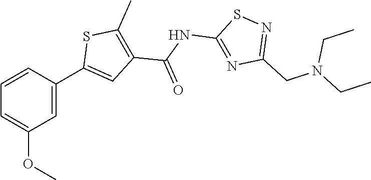 [SMILES: CCN(CC)Cc1nsc(NC(=O)c2cc(-c3cccc(OC)c3)sc2C)n1] |
| C-53 | 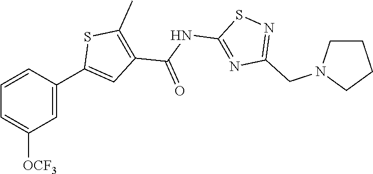 [SMILES: Cc1sc(-c2cccc(OC(F)(F)F)c2)cc1C(=O)Nc1nc(CN2CCCC2)ns1] |
| C-56 | 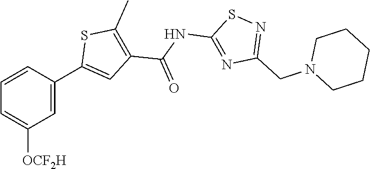 |
| C-57 | 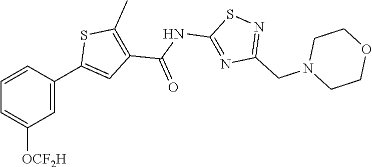 |
| C-58 | 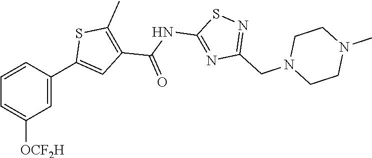 |
| D-1 | 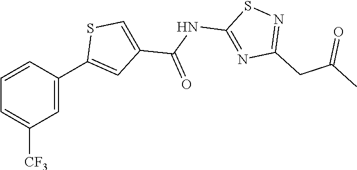 [SMILES: CC(=O)Cc1nsc(NC(=O)c2csc(-c3cccc(C(F)(F)F)c3)c2)n1] |
| D-2 | 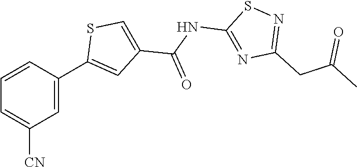 [SMILES: CC(=O)Cc1nsc(NC(=O)c2csc(-c3cccc(C#N)c3)c2)n1] |
| D-3 | 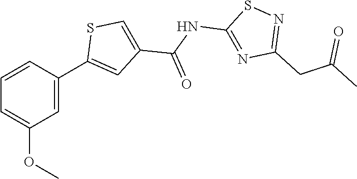 [SMILES: COc1cccc(-c2cc(C(=O)Nc3nc(CC(C)=O)ns3)cs2)c1] |
| D-4 | 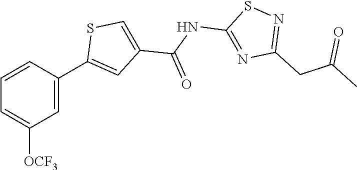 [SMILES: CC(=O)Cc1nsc(NC(=O)c2csc(-c3cccc(OC(F)(F)F)c3)c2)n1] |
| D-5 | 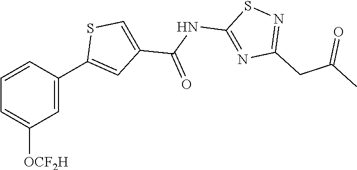 |
| D-6 | 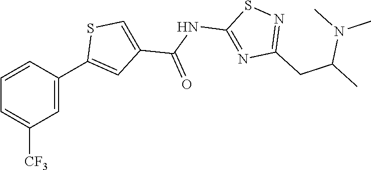 [SMILES: CC(Cc1nsc(NC(=O)c2csc(-c3cccc(C(F)(F)F)c3)c2)n1)N(C)C] |
| D-7 | 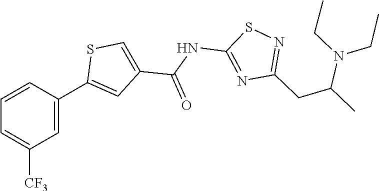 [SMILES: CCN(CC)C(C)Cc1nsc(NC(=O)c2csc(-c3cccc(C(F)(F)F)c3)c2)n1] |
| D-12 | 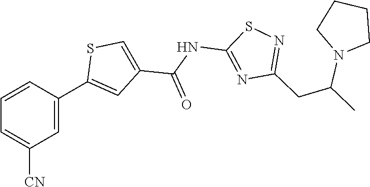 [SMILES: CC(Cc1nsc(NC(=O)c2csc(-c3cccc(C#N)c3)c2)n1)N1CCCC1] |
| D-14 | 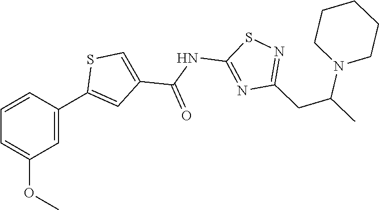 [SMILES: COc1cccc(-c2cc(C(=O)Nc3nc(CC(C)N4CCCCC4)ns3)cs2)c1] |
| D-16 | 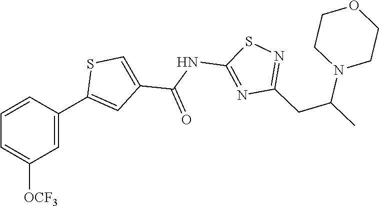 [SMILES: CC(Cc1nsc(NC(=O)c2csc(-c3cccc(OC(F)(F)F)c3)c2)n1)N1CCOCC1] |
| D-19 | 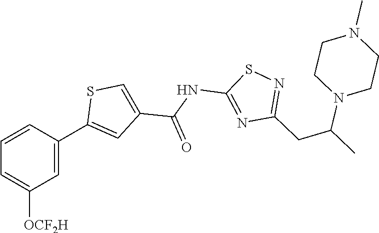 |
| D-20 | 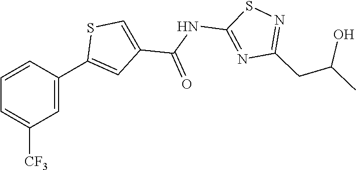 [SMILES: CC(O)Cc1nsc(NC(=O)c2csc(-c3cccc(C(F)(F)F)c3)c2)n1] |
| D-25 | 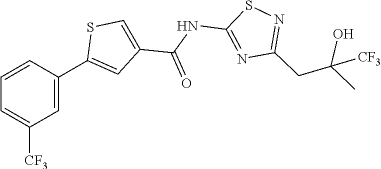 [SMILES: CC(O)(Cc1nsc(NC(=O)c2csc(-c3cccc(C(F)(F)F)c3)c2)n1)C(F)(F)F] |
| D-30 | 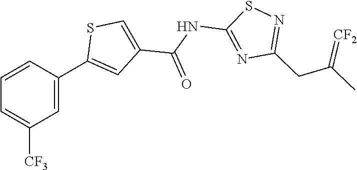 |
| D-35 | 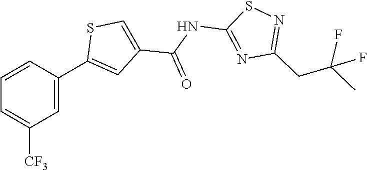 [SMILES: CC(F)(F)Cc1nsc(NC(=O)c2csc(-c3cccc(C(F)(F)F)c3)c2)n1] |
| D-40 | 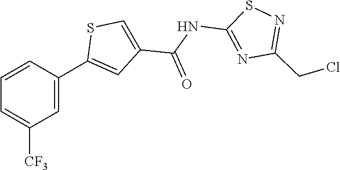 [SMILES: O=C(Nc1nc(CCl)ns1)c1csc(-c2cccc(C(F)(F)F)c2)c1] |
| D-47 | 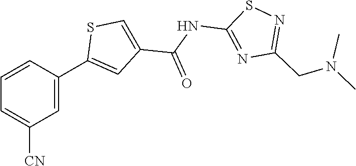 [SMILES: CN(C)Cc1nsc(NC(=O)c2csc(-c3cccc(C#N)c3)c2)n1] |
| D-50 | 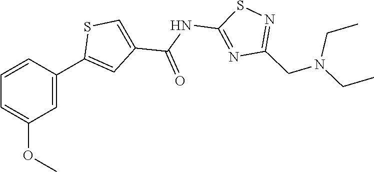 [SMILES: CCN(CC)Cc1nsc(NC(=O)c2csc(-c3cccc(OC)c3)c2)n1] |
| D-53 | 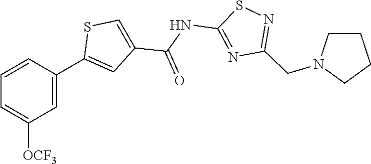 [SMILES: O=C(Nc1nc(CN2CCCC2)ns1)c1csc(-c2cccc(OC(F)(F)F)c2)c1] |
| D-56 | 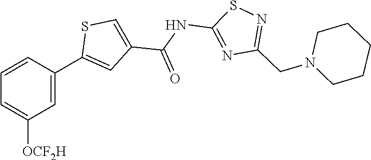 |
| D-57 | 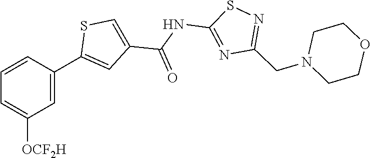 |
| D-58 | 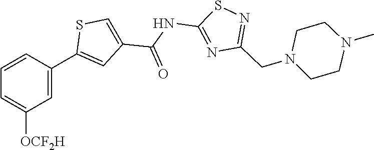 |
| E-1 | 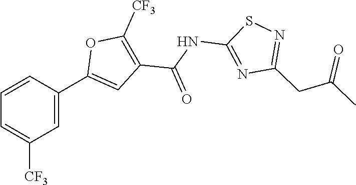 [SMILES: CC(=O)Cc1nsc(NC(=O)c2cc(-c3cccc(C(F)(F)F)c3)oc2C(F)(F)F)n1] |
| E-2 | 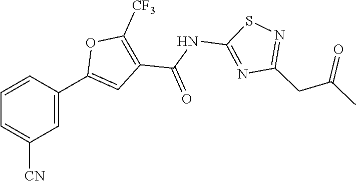 [SMILES: CC(=O)Cc1nsc(NC(=O)c2cc(-c3cccc(C#N)c3)oc2C(F)(F)F)n1] |
| E-3 | 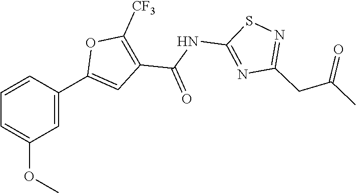 [SMILES: COc1cccc(-c2cc(C(=O)Nc3nc(CC(C)=O)ns3)c(C(F)(F)F)o2)c1] |
| E-4 | 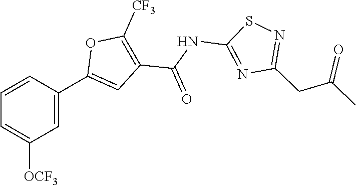 [SMILES: CC(=O)Cc1nsc(NC(=O)c2cc(-c3cccc(OC(F)(F)F)c3)oc2C(F)(F)F)n1] |
| E-5 | 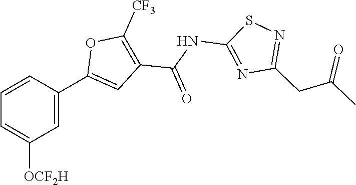 |
| E-6 | 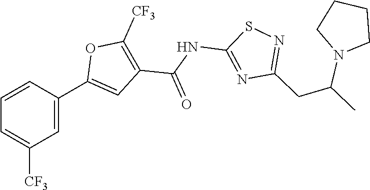 [SMILES: CC(Cc1nsc(NC(=O)c2cc(-c3cccc(C(F)(F)F)c3)oc2C(F)(F)F)n1)N1CCCC1] |
| E-8 | 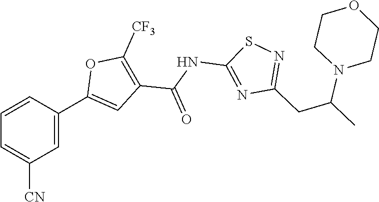 [SMILES: CC(Cc1nsc(NC(=O)c2cc(-c3cccc(C#N)c3)oc2C(F)(F)F)n1)N1CCOCC1] |
| E-9 | 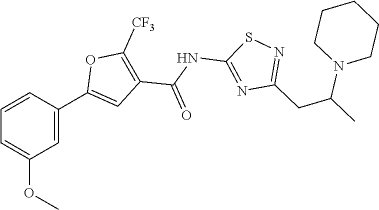 [SMILES: COc1cccc(-c2cc(C(=O)Nc3nc(CC(C)N4CCCCC4)ns3)c(C(F)(F)F)o2)c1] |
| E-10 | 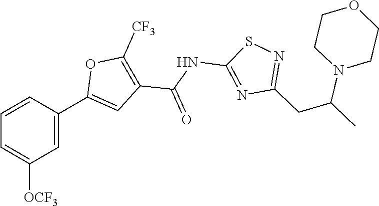 [SMILES: CC(Cc1nsc(NC(=O)c2cc(-c3cccc(OC(F)(F)F)c3)oc2C(F)(F)F)n1)N1CCOCC1] |
| E-11 | 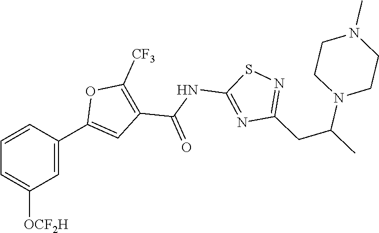 |
| E-12 | 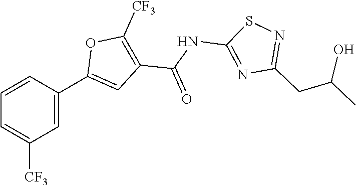 [SMILES: CC(O)Cc1nsc(NC(=O)c2cc(-c3cccc(C(F)(F)F)c3)oc2C(F)(F)F)n1] |
| E-17 | 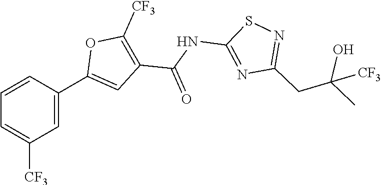 [SMILES: CC(O)(Cc1nsc(NC(=O)c2cc(-c3cccc(C(F)(F)F)c3)oc2C(F)(F)F)n1)C(F)(F)F] |
| E-22 | 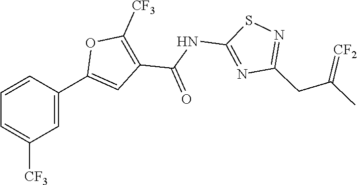 |
| E-27 | 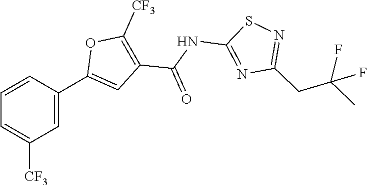 [SMILES: CC(F)(F)Cc1nsc(NC(=O)c2cc(-c3cccc(C(F)(F)F)c3)oc2C(F)(F)F)n1] |
| E-32 | 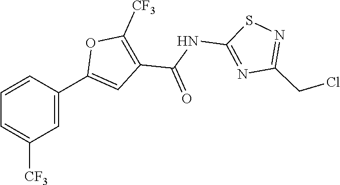 [SMILES: O=C(Nc1nc(CCl)ns1)c1cc(-c2cccc(C(F)(F)F)c2)oc1C(F)(F)F] |
| E-38 | 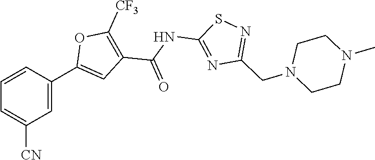 [SMILES: CN1CCN(Cc2nsc(NC(=O)c3cc(-c4cccc(C#N)c4)oc3C(F)(F)F)n2)CC1] |
| E-39 | 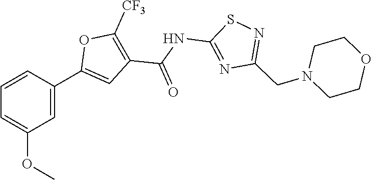 [SMILES: COc1cccc(-c2cc(C(=O)Nc3nc(CN4CCOCC4)ns3)c(C(F)(F)F)o2)c1] |
| E-40 | 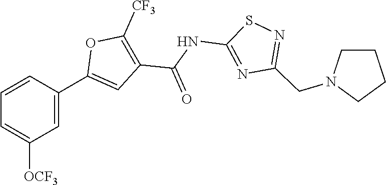 [SMILES: O=C(Nc1nc(CN2CCCC2)ns1)c1cc(-c2cccc(OC(F)(F)F)c2)oc1C(F)(F)F] |
| E-41 | 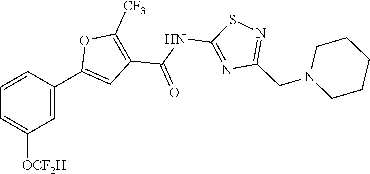 |
| F-1 | 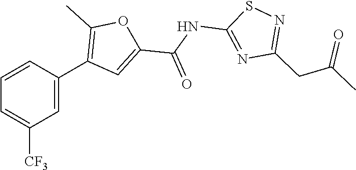 [SMILES: CC(=O)Cc1nsc(NC(=O)c2cc(-c3cccc(C(F)(F)F)c3)c(C)o2)n1] |
| F-2 | 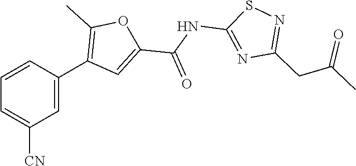 [SMILES: CC(=O)Cc1nsc(NC(=O)c2cc(-c3cccc(C#N)c3)c(C)o2)n1] |
| F-3 | 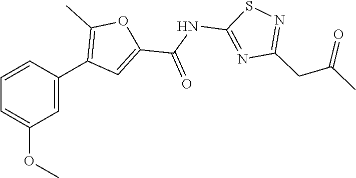 [SMILES: COc1cccc(-c2cc(C(=O)Nc3nc(CC(C)=O)ns3)oc2C)c1] |
| F-4 | 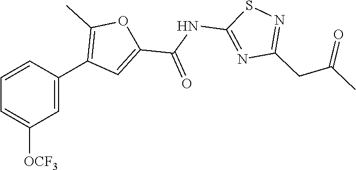 [SMILES: CC(=O)Cc1nsc(NC(=O)c2cc(-c3cccc(OC(F)(F)F)c3)c(C)o2)n1] |
| F-5 | 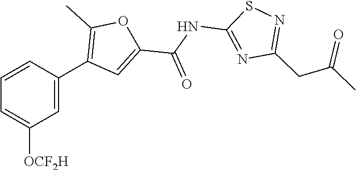 |
| F-6 | 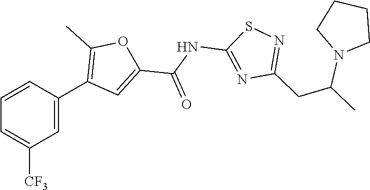 [SMILES: Cc1oc(C(=O)Nc2nc(CC(C)N3CCCC3)ns2)cc1-c1cccc(C(F)(F)F)c1] |
| F-7 | 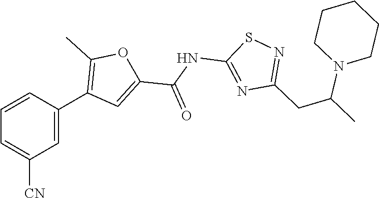 [SMILES: Cc1oc(C(=O)Nc2nc(CC(C)N3CCCCC3)ns2)cc1-c1cccc(C#N)c1] |
| F-8 | 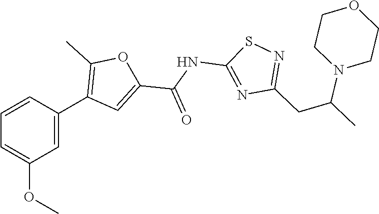 [SMILES: COc1cccc(-c2cc(C(=O)Nc3nc(CC(C)N4CCOCC4)ns3)oc2C)c1] |
| F-9 | 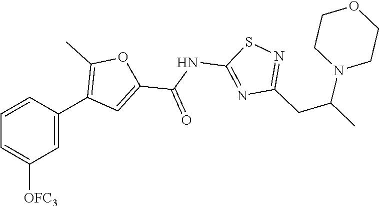 |
| F-10 | 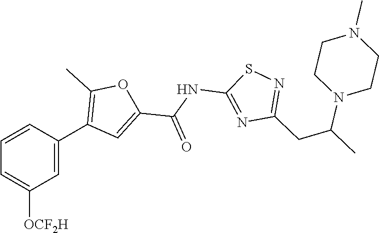 |
| F-11 | 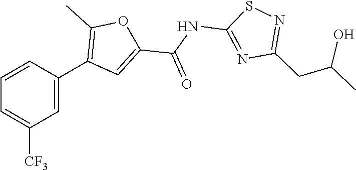 [SMILES: Cc1oc(C(=O)Nc2nc(CC(C)O)ns2)cc1-c1cccc(C(F)(F)F)c1] |
| F-13 | 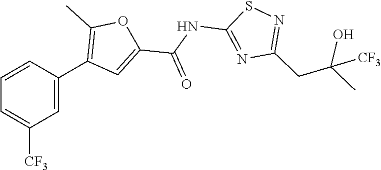 [SMILES: Cc1oc(C(=O)Nc2nc(CC(C)(O)C(F)(F)F)ns2)cc1-c1cccc(C(F)(F)F)c1] |
| F-15 | 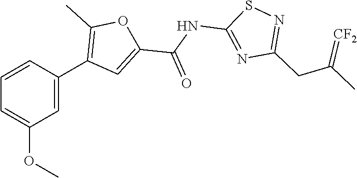 |
| F-17 | 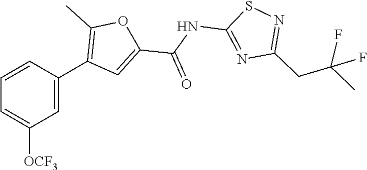 [SMILES: Cc1oc(C(=O)Nc2nc(CC(C)(F)F)ns2)cc1-c1cccc(OC(F)(F)F)c1] |
| F-19 | 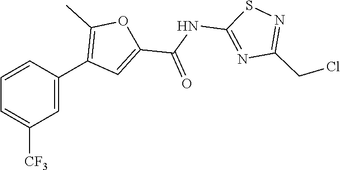 [SMILES: Cc1oc(C(=O)Nc2nc(CCl)ns2)cc1-c1cccc(C(F)(F)F)c1] |
| F-24 | 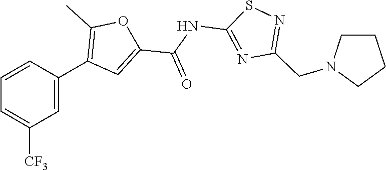 [SMILES: Cc1oc(C(=O)Nc2nc(CN3CCCC3)ns2)cc1-c1cccc(C(F)(F)F)c1] |
| F-25 | 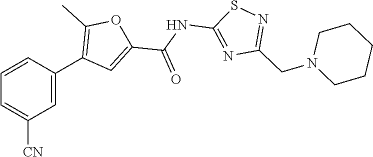 [SMILES: Cc1oc(C(=O)Nc2nc(CN3CCCCC3)ns2)cc1-c1cccc(C#N)c1] |
| F-26 | 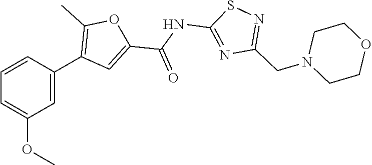 [SMILES: COc1cccc(-c2cc(C(=O)Nc3nc(CN4CCOCC4)ns3)oc2C)c1] |
| F-27 | 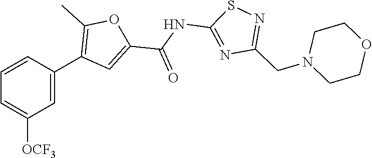 [SMILES: Cc1oc(C(=O)Nc2nc(CN3CCOCC3)ns2)cc1-c1cccc(OC(F)(F)F)c1] |
| F-28 | 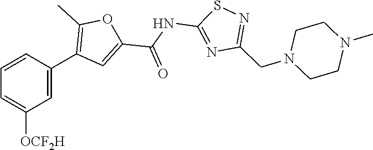 |
| G-1 | 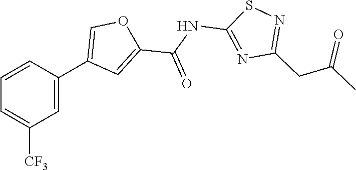 [SMILES: CC(=O)Cc1nsc(NC(=O)c2cc(-c3cccc(C(F)(F)F)c3)co2)n1] |
| G-2 | 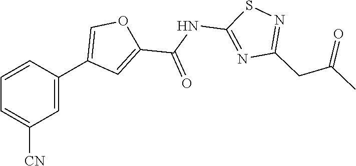 [SMILES: CC(=O)Cc1nsc(NC(=O)c2cc(-c3cccc(C#N)c3)co2)n1] |
| G-3 | 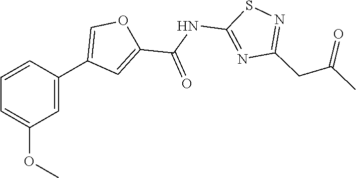 [SMILES: COc1cccc(-c2coc(C(=O)Nc3nc(CC(C)=O)ns3)c2)c1] |
| G-4 | 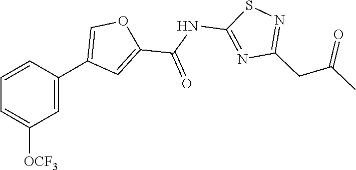 [SMILES: CC(=O)Cc1nsc(NC(=O)c2cc(-c3cccc(OC(F)(F)F)c3)co2)n1] |
| G-5 | 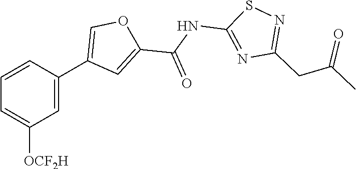 |
| G-6 | 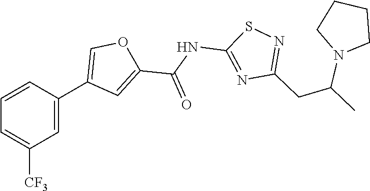 [SMILES: CC(Cc1nsc(NC(=O)c2cc(-c3cccc(C(F)(F)F)c3)co2)n1)N1CCCC1] |
| G-7 | 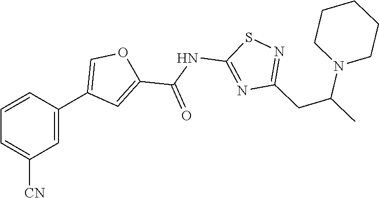 [SMILES: CC(Cc1nsc(NC(=O)c2cc(-c3cccc(C#N)c3)co2)n1)N1CCCCC1] |
| G-8 | 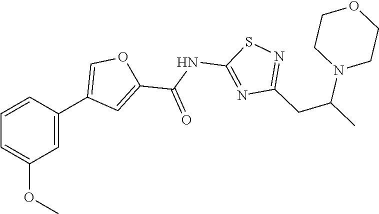 [SMILES: COc1cccc(-c2coc(C(=O)Nc3nc(CC(C)N4CCOCC4)ns3)c2)c1] |
| G-9 | 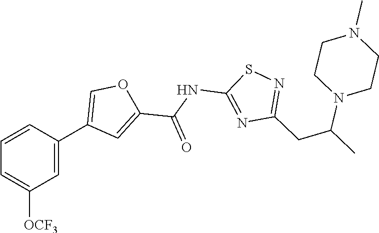 [SMILES: CC(Cc1nsc(NC(=O)c2cc(-c3cccc(OC(F)(F)F)c3)co2)n1)N1CCN(C)CC1] |
| G-10 | 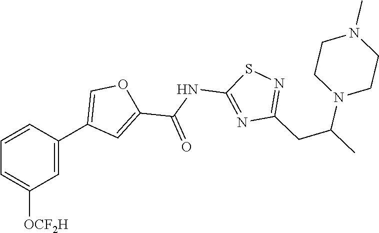 |
| G-11 | 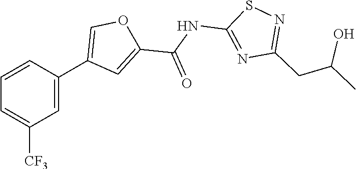 [SMILES: CC(O)Cc1nsc(NC(=O)c2cc(-c3cccc(C(F)(F)F)c3)co2)n1] |
| G-13 | 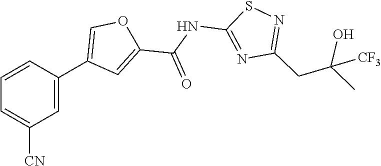 [SMILES: CC(O)(Cc1nsc(NC(=O)c2cc(-c3cccc(C#N)c3)co2)n1)C(F)(F)F] |
| G-15 | 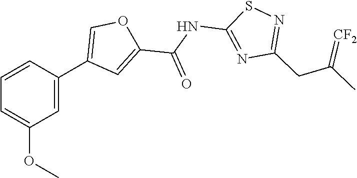 |
| G-17 | 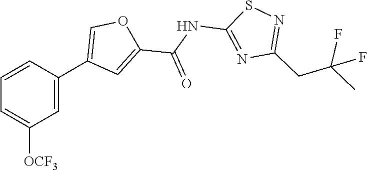 [SMILES: CC(F)(F)Cc1nsc(NC(=O)c2cc(-c3cccc(OC(F)(F)F)c3)co2)n1] |
| G-19 | 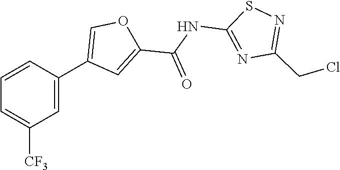 [SMILES: O=C(Nc1nc(CCl)ns1)c1cc(-c2cccc(C(F)(F)F)c2)co1] |
| G-24 | 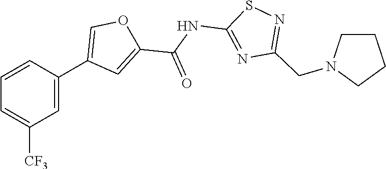 [SMILES: O=C(Nc1nc(CN2CCCC2)ns1)c1cc(-c2cccc(C(F)(F)F)c2)co1] |
| G-25 | 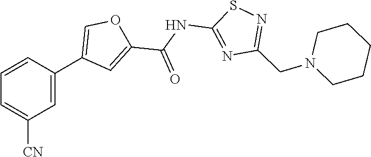 [SMILES: N#Cc1cccc(-c2coc(C(=O)Nc3nc(CN4CCCCC4)ns3)c2)c1] |
| G-26 | 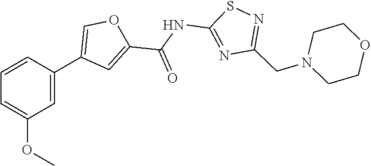 [SMILES: COc1cccc(-c2coc(C(=O)Nc3nc(CN4CCOCC4)ns3)c2)c1] |
| G-27 | 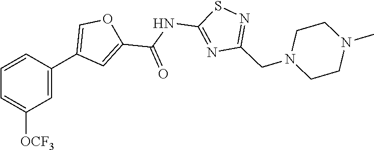 [SMILES: CN1CCN(Cc2nsc(NC(=O)c3cc(-c4cccc(OC(F)(F)F)c4)co3)n2)CC1] |
| G-28 | 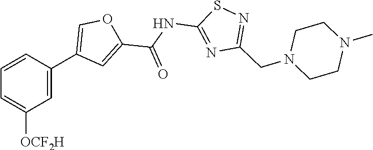 |

The present invention also provides stereoisomers of the thiadiazoleamide compounds, as well as prodrugs, pharmaceutically acceptable salts, solvates, and deuterated products.

The term “solvate” refers to complexes formed by solute (compounds provided by formula (I)) and solvents. The solvents (pharmaceutically acceptable solvents) include, but are not limited to water, methanol, ethanol, acetic acid, etc.

The present invention also provides “polymorphs” include, but not limited to compounds provided by formula (I), or their salts, or their solvates. For example, the polymorphs possess different crystalline forms and have different physical properties such as density, stability and solubility, etc.

The thiadiazoleamide compounds provided by formula (I) exhibited excellent AR antagonistic activities and effectively inhibits the proliferation of tumor cells with high safety profiles. Mechanism research has demonstrated that these compounds are capable of blocking AR dimerization by binding to the AR-LBD dimer interface. Therefore, the thiadiazoleamide compounds can be applied to the treatment of tumors related to abnormal AR expression or mutations.

Specifically, the applications of the present invention provided stereoisomers of the thiadiazoleamide compounds, or prodrugs, or pharmaceutically acceptable salts, or solvate, or deuterated products include, but not limited to the treatment of PCa, metastatic PCa, castration-resistant PCa, breast cancer and ovarian cancer.

The therapeutic mechanism of the thiadiazoleamide compounds includes: its active ingredient thiadiazole amide derivative inhibits the proliferation of tumor cells by antagonizing the activity of the androgen receptor, further, the thiadiazole amide derivative blocks the dimerization of the androgen receptor ligand domain and thus Play an antagonistic role on the androgen receptor.

The present invention also provides a pharmaceutical composition for the treatment of AR abnormal expression or mutations related tumors, which contains a therapeutically effective amount of one or more compounds provided by formula (I), or pharmaceutically acceptable salts, stereoisomers, prodrugs, or deuterated products, along with pharmaceutically acceptable carriers.

The active component of the pharmaceutical preparation is one or more of compounds provided by formula (I), or pharmaceutically acceptable salts, or stereoisomers, or prodrugs, or deuterated products. The carriers commonly used in pharmaceutical field include, but not limited to excipient, filler, disintegrant, diluent, surfactant, absorption enhancer, adsorption carrier, binder, lubricant, humectant, flavoring agent, sweetener, etc. The pharmaceutical preparation of the present invention can be prepared according to conventional methods in the pharmaceutical field, include, but not limited to capsules, granules, powders, tablets, oral liquids, injections, etc.

The term “pharmaceutically acceptable salts” refers to salts prepared by conventional methods, including, but not limited to organic acid salts, inorganic acid salts, organic base salts, inorganic base salts. The organic acid salts include but not limited to oxalate, lactate, tosilate, malate, citrate, fumarate, camphorsulfonate, methanesulfonate, etc. The inorganic acid salts include, but not limited to nitrates, sulfates, hydrohalites, phosphates, etc. The organic base salts include, but not limited to sodium methoxide, potassium methoxide, sodium ethoxide, potassium ethoxide, triethylamine, tert-butylamine, etc. The inorganic base salts include, but not limited to sodium hydroxide, potassium hydroxide, lithium hydroxide, etc.

The compounds provided by the present invention can be used in combination with antitumor drugs including, but not limited to antitubulin drugs (e.g., taxol), antimitotic drugs (e.g., vinblastine, vindesine, etc.), antimetabolites (e.g., 5-fluorouracil, methotrexate, etc.), alkylating agents (cisplatin, carboplatin, etc.), antibiotic drugs (e.g., mitomycin, etc.), etc.

The present invention has the following beneficial effects:

The present invention provides a novel class of thiadiazoleamide compounds exhibiting excellent AR antagonistic activities. These compounds effectively block AR dimerization by binding to the AR-LBD dimer interface. The compounds provided by the present invention possess high AR antagonistic activity at the molecular, cellular and animal levels, with high safety profiles. Therefore, these compounds can be applied to the preparation of medicaments for the treatment of tumors associated with abnormal AR expression, including but not limited to PCa, metastatic PCa, castration-resistant PCa, breast cancer and ovarian cancer.

### BRIEF DESCRIPTION OF THE DRAWINGS

FIG. 1 is the synthesis route of compounds A-1˜A-23.

FIG. 2 is the synthesis route of compound A-1.

FIG. 3 is the synthesis route of compounds A-24˜A-30.

FIG. 4 is the synthesis route of compound A-31.

FIG. 5 is the synthesis route of compounds A-34˜A-37.

FIG. 6 is the synthesis route of compound A-34.

FIG. 7 is the synthesis route of compounds A-38˜A-43.

FIG. 8 is the synthesis route of compound A-38.

FIG. 9 is the synthesis route of compounds A-60˜A-61.

FIG. 10 is the synthesis route of compound A-66.

FIG. 11 is the synthesis route of compounds A-69˜A-70.

FIG. 12 is the synthesis route of compound A-75.

FIG. 13 is the synthesis route of compound A-80.

FIG. 14 is the synthesis route of compound A-85.

FIG. 15 is the synthesis route of compound A-90.

FIG. 16 is the synthesis route of compound A-93.

FIG. 17 is the synthesis route of compound A-96.

FIG. 18 is the synthesis route of compounds A-99˜A-104.

FIG. 19 is the synthesis route of compound A-99.

FIG. 20 is the synthesis route of compounds A-105˜A-111.

FIG. 21 is the synthesis route of compounds A-105 and A-106.

FIG. 22 is the synthesis route of compounds B-1˜B-5.

FIG. 23 is the synthesis route of compound B-1.

FIG. 24 is the synthesis route of compounds B-6˜B-19.

FIG. 25 is the synthesis route of compounds B-20˜B-24.

FIG. 26 is the synthesis route of compounds B-25˜B-29.

FIG. 27 is the synthesis route of compounds B-30˜B-34.

FIG. 28 is the synthesis route of compounds B-35˜B-39.

FIG. 29 is the synthesis route of compounds B-40˜B-58.

FIG. 30 is the synthesis route of compounds C-1˜C-5.

FIG. 31 is the synthesis route of compound C-1.

FIG. 32 is the synthesis route of compounds C-6˜C-19.

FIG. 33 is the synthesis route of compounds C-20˜C-24.

FIG. 34 is the synthesis route of compounds C-25˜C-29.

FIG. 35 is the synthesis route of compounds C-30˜C-34.

FIG. 36 is the synthesis route of compounds C-35˜C-39.

FIG. 37 is the synthesis route of compounds C-40˜C-58.

FIG. 38 is the synthesis route of compounds D-1˜D-5.

FIG. 39 is the synthesis route of compound D-1.

FIG. 40 is the synthesis route of compounds D-6˜D-19.

FIG. 41 is the synthesis route of compounds D-20˜D-24.

FIG. 42 is the synthesis route of compounds D-25˜D-29.

FIG. 43 is the synthesis route of compounds D-30˜D-34.

FIG. 44 is the synthesis route of compounds D-35˜D-39.

FIG. 45 is the synthesis route of compounds D-40˜D-58.

FIG. 46 is the synthesis route of compounds E-1˜E-5.

FIG. 47 is the synthesis route of compound E-1.

FIG. 48 is the synthesis route of compounds E-6˜E-11.

FIG. 49 is the synthesis route of compounds E-12˜E-16.

FIG. 50 is the synthesis route of compounds E-17˜E-21.

FIG. 51 is the synthesis route of compounds E-22˜E-26.

FIG. 52 is the synthesis route of compounds E-27˜E-31.

FIG. 53 is the synthesis route of compounds E-32˜E-42.

FIG. 54 is the synthesis route of compounds F-1˜F-5.

FIG. 55 is the synthesis route of compound F-1.

FIG. 56 is the synthesis route of compounds F-6˜F-10.

FIG. 57 is the synthesis route of compounds F-11˜F-12.

FIG. 58 is the synthesis route of compounds F-13˜F-14.

FIG. 59 is the synthesis route of compounds F-15˜F-16.

FIG. 60 is the synthesis route of compounds F-17˜F-18.

FIG. 61 is the synthesis route of compounds F-19˜F-28.

FIG. 62 is the synthesis route of compounds G-1˜G-5.

FIG. 63 is the synthesis route of compound G-1.

FIG. 64 is the synthesis route of compounds G-6˜G-10.

FIG. 65 is the synthesis route of compounds G-11˜G-12.

FIG. 66 is the synthesis route of compounds G-13˜G-14.

FIG. 67 is the synthesis route of compounds G-15˜G-16.

FIG. 68 is the synthesis route of compounds G-17˜G-18.

FIG. 69 is the synthesis route of compounds G-19˜G-28.

FIG. 70 is the result of compound A-1 has no ability to binding to the LBP of AR-LBD.

FIG. 71 is the result of biolayer interferometry (BLI) assay shows direct interaction between compound A-1 and AR-LBD.

FIG. 72 is the result of small-angle X-ray scattering (SAXS) shows compound A-1 is capable of blocking AR-LBD dimerazation.

FIG. 73 is the result of ARV684A shows enhanced AR antagonistic activity for compound A-1.

FIG. 74 is the result of compound A-1 inhibiting tumor volume in LNCaP xenograft model.

FIG. 75 is the result of compound A-1 inhibiting tumor weight in LNCaP xenograft model.

FIG. 76 is the result of compound A-1 possesses no mortality or significant loss of body weight in LNCaP xenograft model.

### DETAILED DESCRIPTION OF THE EMBODIMENTS OF THE INVENTION

The present invention will be further described upon in the following examples for reference. The examples are utilized to illustration, and should not be interpreted as the limitation of the invention.

In addition, it should be understood that various changes and modifications of the present invention may be made by skilled technicist after reading the present invention, and such changes and modifications are equal to the scope of the present invention.

The experimental procedures used in the following examples are all conventional procedures unless specifically stated.

Materials, reagents, etc., used in the following examples are commercially available unless specifically stated.

### Example 1: Synthesis of Compounds A-1˜A-23

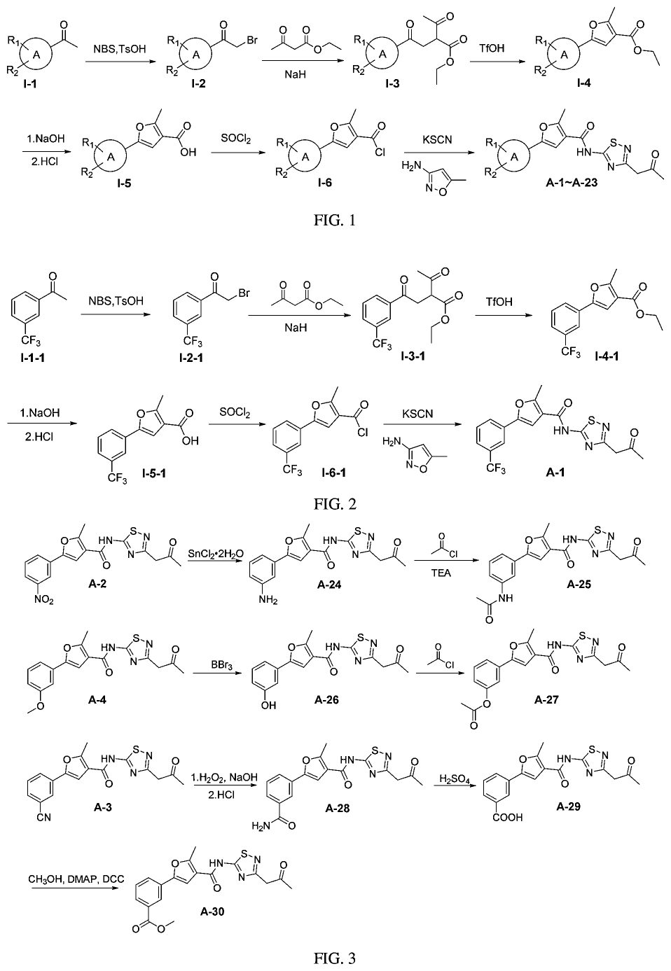

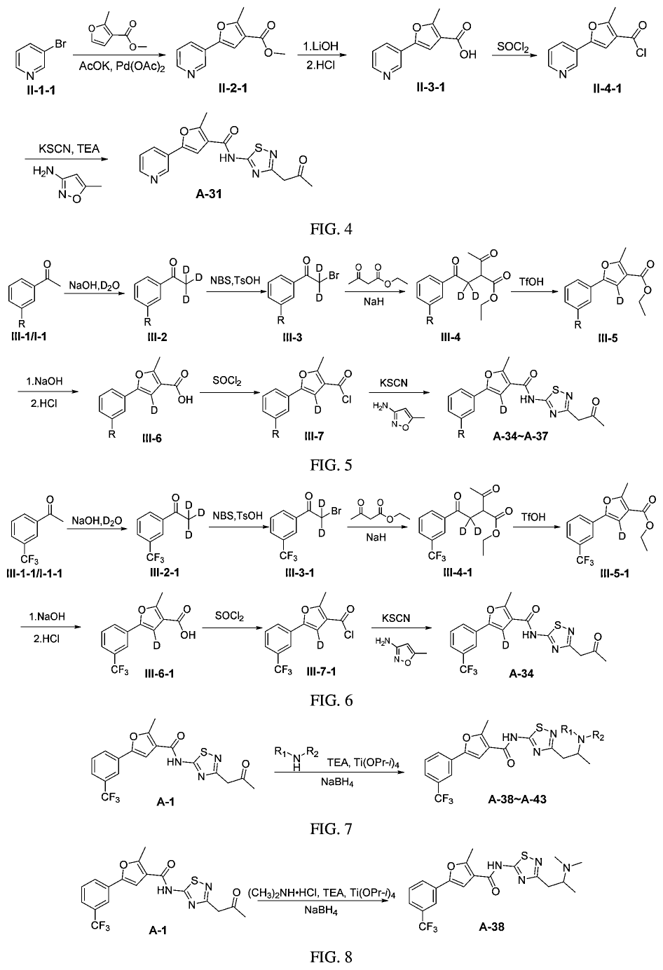

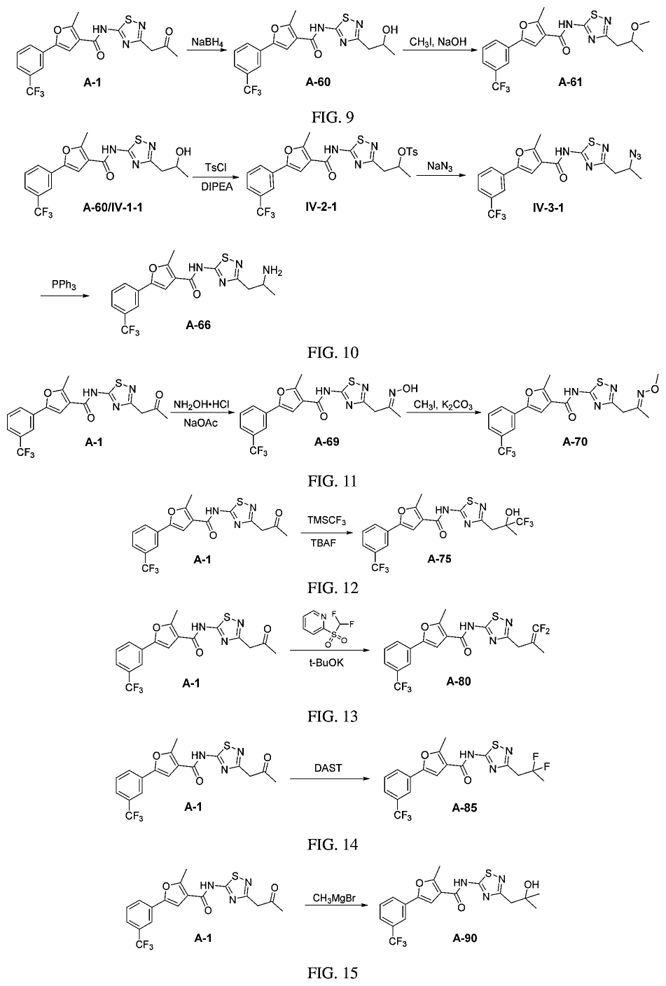

General synthetic route of compounds A-1˜A-23 is shown in FIG. 1.

### 2-Methyl-N-(3-(2-oxopropyl)-1,2,4-thiadiazol-5-yl)-5-(3-(trifluoromethyl)phenyl)furan-3-carboxamide (Compound A-1)

The synthetic route of compound A-1 is shown in FIG. 2.

### (a) Synthesis of 2-bromo-1-(3-(trifluoromethyl)phenyl)ethan-1-one (Compound I-2-1)

To a stirring suspension of NBS (1.04 g, 5.9 mmol) and p-Toluenesulfonic acid (458 mg, 2.7 mmol) in 20 mL acetonitrile was added 1-(3-(trifluoromethyl)phenyl)ethan-1-one (I-1-1, 1.00 g, 5.3 mmol). This solution was stirred at 55° C. for 1 hour, and ethyl acetate was added. The organic layer was washed with water and brine, and then dried over anhydrous Na₂SO₄, and concentrated to give a residue, which was purified by column-chromatograph to give a light yellow oil, yield was 88%; ESA-MS: m/z=267.0 [M+H]⁺.

### (b) Synthesis of ethyl 2-acetyl-4-oxo-4-(3-(trifluoromethyl)phenyl)butanoate (Compound I-3-1)

To a suspension of sodium hydride (60% w/w 197 mg, 4.7 mmol) in 20 mL anhydrous THF was added ethyl acetate (439 mg, 4.2 mmol) by drop at 0° C. This solution was stirred at 0° C. for 30 min and compound I-2-1 (1.25 g, 4.7 mmol) was added. Subsequently, the solution was warmed to room temperature, stirred overnight and then quenched with water. The mixture was extracted with ethyl acetate, and the combined organic layer was washed with brine, dried over anhydrous Na₂SO₄, and concentrated to give a residue, which was purified by column-chromatograph to give a light yellow solid, yield was 71%; ESA-MS: m/z=317.1 [M+H]⁺.

### (c) Synthesis of ethyl 2-methyl-5-(3-(trifluoromethyl)phenyl)furan-3-carboxylate (Compound I-4-1)

To solution of compound I-3-1 (946 mg, 3.0 mmol) in 20 mL acetonitrile was added trifluoro-methanesulfonic acid (449 mg, 3.0 mmol) under nitrogen atmosphere. This solution was heated to 85° C. for 1 hour, and then ethyl acetate was added. The mixture was washed with water and brine, then dried over anhydrous Na₂SO₄, and concentrated to give a residue, which was purified by column-chromatograph to give a light yellow solid, yield was 78%; ESA-MS: m/z=299.1 [M+H]⁺.

### (d) Synthesis of 2-methyl-5-(3-(trifluoromethyl)phenyl)furan-3-carboxylic acid (Compound I-5-1)

To a solution of compound I-4-1 (699 mg, 2.3 mmol) in EtOH/H₂O (3:1, 20 mL) was added sodium hydroxide (584 mg, 14.6 mmol) and stirred overnight at room temperature. The reaction mixture was adjusted with 2 mol/L HCl to acidity, and ethyl acetate was added. The mixture was washed with water and brine, dried over anhydrous Na₂SO₄, and concentrated to give a light yellow solid, yield was 90%; ESA-MS: m/z=271.1 [M+H]⁺.

### (c) Synthesis of 2-methyl-5-(3-(trifluoromethyl)phenyl)furan-3-carbonyl chloride (Compound I-6-1)

A solution of compound I-5-1 (584 mg, 2.2 mmol) in 10 mL SOCl₂ was heated to reflux for 2.5 hours, and the mixture was concentrated to dryness to get a light brown solid, yield was 95%;

### (f) Synthesis of 2-methyl-N-(3-(2-oxopropyl)-1,2,4-thiadiazol-5-yl)-5-(3-(trifluoromethyl)phenyl)furan-3-carboxamide (Compound A-1)

A suspension of compound I-6-1 (620 mg, 2.2 mmol) and potassium thiocyanate (230 mg, 2.4 mmol) in 15 mL acetonitrile was stirred at 60° C. for 30 min. and then 5-methylisoxazol-3-amine (210 mg, 2.2 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 81%; ¹HNMR (500 MHZ, DMSO-d6): δ 12.72 (s, 1H), 8.09-8.07 (m, 2H), 7.69-7.66 (m, 1H), 7.48-7.44 (m, 1H), 6.81 (s, 1H), 3.71 (s, 2H), 2.57 (s, 3H), 1.89 (s, 3H); ESA-MS: m/z=410.1 [M+H]⁺.

### 2-Methyl-5-(3-nitrophenyl)-N-(3-(2-oxopropyl)-1,2,4-thiadiazol-5-yl)furan-3-carboxamide (Compound A-2)

Preparation of compound A-2 referred to the synthesis of compound A-1, replacing m-trifluoromethyl acetophenone with 3-nitroacetophenone to obtain a white solid, yield was 33%; ¹HNMR (500 MHZ, Chloroform-d): δ 8.52-8.50 (m, 1H), 8.19-8.16 (m, 1H), 7.98-7.94 (m, 1H), 7.62 (t, J=8.0 Hz, 1H), 7.12 (s, 1H), 4.04 (s, 2H), 2.81 (s, 3H), 2.29 (s, 3H); ESA-MS: m/z=387.1 [M+H]⁺.

### 5-(3-Cyanophenyl)-2-methyl-N-(3-(2-oxopropyl)-1,2,4-thiadiazol-5-yl)furan-3-carboxamide (Compound A-3)

Preparation of compound A-3 referred to the synthesis of compound A-1, replacing m-trifluoromethyl acctophenone with 3-cyanophenone to obtain a white solid, yield was 35%; ¹H NMR (500 MHZ, Chloroform-d): δ 8.03 (t, J=1.5 Hz, 1H), 7.91-7.87 (m, 1H), 7.69-7.65 (m, 1H), 7.50 (t, J=7.5 Hz, 1H), 7.22 (s, 1H), 3.59 (s, 2H), 2.54 (s, 3H), 2.28 (s, 3H); ESA-MS: m/z=367.1 [M+H]⁺.

### 5-(3-Methoxyphenyl)-2-methyl-N-(3-(2-oxopropyl)-1,2,4-thiadiazol-5-yl)furan-3-carboxamide (Compound A-4)

Preparation of compound A-4 referred to the synthesis of compound A-1, replacing m-trifluoromethyl acetophenone with 3-methoxyacetophenone to obtain a light yellow solid, yield was 33%; ¹HNMR (500 MHZ, DMSO-d6): δ 13.15 (s, 1H), 7.70 (s, 1H), 7.40 (t, J=7.5 Hz, 1H), 7.24-7.20 (m, 1H), 7.16-7.14 (m, 1H), 6.96-6.92 (m, 1H), 4.04 (s, 2H), 3.82 (s, 3H), 2.70 (s, 3H), 2.20 (s, 3H); ESA-MS: m/z=372.1 [M+H]⁺.

### 2-Methyl-N-(3-(2-oxopropyl)-1,2,4-thiadiazol-5-yl)-5-phenylfuran-3-carboxamide (Compound A-5)

Preparation of compound A-5 referred to the synthesis of compound A-1, replacing m-trifluoromethyl acctophenone with acetophenone to obtain a light yellow solid, yield was 34%; ¹HNMR (500 MHZ, DMSO-d6): δ 13.16 (s, 1H), 7.68 (s, 1H), 7.66-7.62 (m, 2H), 7.52-7.46 (m, 2H), 7.39-7.34 (m, 1H), 4.04 (s, 2H), 2.70 (s, 3H), 2.20 (s, 3H); ESA-MS: m/z=342.1 [M+H]⁺.

### 5-(3-Ethoxyphenyl)-2-methyl-N-(3-(2-oxopropyl)-1,2,4-thiadiazol-5-yl)furan-3-carboxamide (Compound A-6)

Preparation of compound A-6 referred to the synthesis of compound A-1, replacing m-trifluoromethyl acetophenone with 3-ethoxyacetophenone to obtain a white solid, yield was 37%; ¹H NMR (500 MHZ, Chloroform-d): δ 7.62-7.58 (m, 1H), 7.33 (t, J=7.5 Hz, 1H), 7.19 (t, J=1.5 Hz, 1H), 6.99 (s, 1H), 6.96-6.92 (m, 1H), 4.08 (q, J=8.0 Hz, 2H), 3.57 (s, 2H), 2.62 (s, 3H), 2.29 (s, 3H), 1.46 (t, J=8.0 Hz, 3H); ESA-MS: m/z=386.1 [M+H]⁺.

### 5-(3-Fluorophenyl)-2-methyl-N-(3-(2-oxopropyl)-1,2,4-thiadiazol-5-yl)furan-3-carboxamide (Compound A-7)

Preparation of compound A-7 referred to the synthesis of compound A-1, replacing m-trifluoromethyl acetophenone with 3-fluoroacetophenone to obtain a light yellow solid, yield was 40%; ¹H NMR (500 MHZ, Chloroform-d): δ 7.61-7.57 (m, 1H), 7.52-7.47 (m, 1H), 7.45-7.41 (m, 1H), 7.22-7.18 (m, 1H), 7.02 (s, 1H), 3.50 (s, 2H), 2.54 (s, 3H), 2.32 (s, 3H); ESA-MS: m/z=360.1 [M+H]⁺.

### 5-(3-Chlorophenyl)-2-methyl-N-(3-(2-oxopropyl)-1,2,4-thiadiazol-5-yl)furan-3-carboxamide (Compound A-8)

Preparation of compound A-8 referred to the synthesis of compound A-1, replacing m-trifluoromethyl acetophenone with 3-chloroacetophenone to obtain a white solid, yield was 40%; ¹HNMR (500 MHZ, Chloroform-d): δ 7.67-7.64 (m, 1H), 7.54-7.50 (m, 1H), 7.36 (t, J=8.0 Hz, 1H), 7.32-7.28 (m, 1H), 6.88 (s, 1H), 4.02 (s, 2H), 2.77 (s, 3H), 2.28 (s, 3H); ESA-MS: m/z=376.0 [M+H]⁺.

### 5-(3-Bromophenyl)-2-methyl-N-(3-(2-oxopropyl)-1,2,4-thiadiazol-5-yl)furan-3-carboxamide (Compound A-9)

Preparation of compound A-9 referred to the synthesis of compound A-1, replacing m-trifluoromethyl acetophenone with 3-bromoacetophenone to obtain a light yellow solid, yield was 38%; ¹HNMR (500 MHZ, DMSO-d6): δ13.16 (s, 1H), 7.79-7.76 (m, 2H), 7.64-7.60 (m, 1H), 7.57-7.53 (m, 1H), 7.45 (t, J=8.0 Hz, 1H), 4.04 (s, 2H), 2.70 (s, 3H), 2.20 (s, 3H); ESA-MS: m/z=420.0 [M+H]⁺.

### 5-(3,4-Dichlorophenyl)-2-methyl-N-(3-(2-oxopropyl)-1,2,4-thiadiazol-5-yl)furan-3-carboxamide (Compound A-10)

Preparation of compound A-10 referred to the synthesis of compound A-1, replacing m-trifluoromethyl acetophenone with 3,4-dichloroacetophenone to obtain a white solid, yield was 36%; ¹HNMR (500 MHZ, DMSO-d6): δ 13.18 (s, 1H), 7.82 (d, J=2.0 Hz, 1H), 7.80-7.30 (m, 2H), 7.59 (dd, J₁=8.5 Hz, J₂=2.5 Hz, 1H), 4.04 (s, 2H), 2.70 (s, 3H), 2.21 (s, 3H); ESA-MS: m/z=410.0 [M+H]⁺.

### 2-Methyl-5-(naphthalen-1-yl)-N-(3-(2-oxopropyl)-1,2,4-thiadiazol-5-yl)furan-3-carboxamide (Compound A-11)

Preparation of compound A-11 referred to the synthesis of compound A-1, replacing m-trifluoromethyl acctophenone with 1-naphthlethanoe to obtain a white solid, yield was 31%; ¹H NMR (500 MHZ, Chloroform-d): δ 8.10-8.01 (m, 1H), 7.96-7.90 (m, 2H), 7.89 (dd, J₁=7.5 Hz, J₂=1.5 Hz, 1H), 7.59-7.49 (m. 3H), 7.20 (s, 1H), 3.58 (s, 2H), 2.63 (s, 3H), 2.30 (s, 3H); ESA-MS: m/z=392.1 [M+H]⁺.

### 2-Methyl-5-(naphthalen-2-yl)-N-(3-(2-oxopropyl)-1,2,4-thiadiazol-5-yl)furan-3-carboxamide (Compound A-12)

Preparation of compound A-12 referred to the synthesis of compound A-1, replacing m-trifluoromethyl acctophenone with 2-naphthlethanoe to obtain a white solid, yield was 30%; ¹H NMR (500 MHZ, Chloroform-d): δ 8.20 (d, J=1.5 Hz, 1H), 8.02-8.98 (m, 1H), 8.98-8.92 (m, 1H), 7.92-7.87 (m, 1H), 7.79 (dd, J₁=7.5 Hz, J₂=1.5 Hz, 1H), 7.54 (dd, J₁=5.5 Hz, J₂=3.5 Hz, 2H), 7.03 (s, 1H), 3.56 (s, 2H), 2.64 (s, 3H), 2.29 (s, 3H); ESA-MS: m/z=392.1 [M+H]⁺.

### 2-Methyl-N-(3-(2-oxopropyl)-1,2,4-thiadiazol-5-yl)-5-(3-(trifluoromethoxy)phenyl)furan-3-carboxamide (Compound A-13)

Preparation of compound A-13 referred to the synthesis of compound A-1, replacing m-trifluoromethyl acctophenone with 3-(trifluoromethoxy) acetophenone to obtain a light yellow solid, yield was 35%; ¹HNMR (500 MHZ, DMSO-d6): δ 13.18 (s, 1H), 7.82 (s, 1H), 7.67-7.60 (m, 2H), 7.52 (s, 1H), 7.35 (d, J=7.5 Hz, 1H), 4.04 (s, 2H), 2.71 (s, 3H), 2.21 (s, 3H); ESA-MS: m/z=426.1 [M+H]⁺.

### 5-(3-(Difluoromethoxy)phenyl)-2-methyl-N-(3-(2-oxopropyl)-1,2,4-thiadiazol-5-yl)furan-3-carboxamide (Compound A-14)

Preparation of compound A-14 referred to the synthesis of compound A-1, replacing m-trifluoromethyl acetophenone with 3-(difluoromethoxy) acetophenone to obtain a light yellow solid, yield was 35%; ¹HNMR (500 MHZ, DMSO-d6): δ 13.16 (s, 1H), 7.76 (s, 1H), 7.54 (t, J=1.5 Hz, 1H), 7.52-7.48 (m, 1H), 7.39-7.36 (m, 1H), 7.34 (t, J=75 Hz, 1H), 7.19-7.15 (m, 1H), 4.04 (s, 2H), 2.71 (s, 3H), 2.21 (s, 3H); ESA-MS: m/z=408.1 [M+H]⁺.

### 2-Methyl-N-(3-(2-oxopropyl)-1,2,4-thiadiazol-5-yl)-5-(3-sulfamoylphenyl)furan-3-carboxamide (Compound A-15)

Preparation of compound A-15 referred to the synthesis of compound A-1, replacing m-trifluoromethyl acctophenone with 3-sulfamoylacetophenone to obtain a white solid, yield was 36%; ESA-MS: m/z=421.1 [M+H]⁺.

### 2-Methyl-N-(3-(2-oxopropyl)-1,2,4-thiadiazol-5-yl)-5-(m-tolyl)furan-3-carboxamide (Compound A-16)

Preparation of compound A-16 referred to the synthesis of compound A-1, replacing m-trifluoromethyl acetophenone with 3-methylacetophenone to obtain a white solid, yield was 35%; ESA-MS: m/z=356.1 [M+H]⁺.

### 5-(3-Ethylphenyl)-2-methyl-N-(3-(2-oxopropyl)-1,2,4-thiadiazol-5-yl)furan-3-carboxamide (Compound A-17)

Preparation of compound A-17 referred to the synthesis of compound A-1, replacing m-trifluoromethyl acetophenone with 3-ethylacetophenone to obtain a white solid, yield was 31%; ESA-MS: m/z=370.1 [M+H]⁺.

### 2-Methyl-N-(3-(2-oxopropyl)-1,2,4-thiadiazol-5-yl)-5-(3-propylphenyl)furan-3-carboxamide (Compound A-18)

Preparation of compound A-18 referred to the synthesis of compound A-1, replacing m-trifluoromethyl acetophenone with 3-propylacetophenone to obtain a white solid, yield was 35%; ESA-MS: m/z=384.1 [M+H]⁺.

### 5-(3-Isopropylphenyl)-2-methyl-N-(3-(2-oxopropyl)-1,2,4-thiadiazol-5-yl)furan-3-carboxamide (Compound A-19)

Preparation of compound A-19 referred to the synthesis of compound A-1, replacing m-trifluoromethyl acetophenone with 3-isopropylacetophenone to obtain a white solid, yield was 35%; ESA-MS: m/z=384.1 [M+H]⁺.

### 5-(3-(Tert-butyl)phenyl)-2-methyl-N-(3-(2-oxopropyl)-1,2,4-thiadiazol-5-yl)furan-3-carboxamide (Compound A-20)

Preparation of compound A-20 referred to the synthesis of compound A-1, replacing m-trifluoromethyl acetophenone with 3-(tert-butyl)acetophenone to obtain a white solid, yield was 34%; ESA-MS: m/z=398.2 [M+H]⁺.

### 2-Methyl-5-(3-(methylthio)phenyl)-N-(3-(2-oxopropyl)-1,2,4-thiadiazol-5-yl)furan-3-carboxamide (Compound A-21)

Preparation of compound A-21 referred to the synthesis of compound A-1, replacing m-trifluoromethyl acetophenone with 3-(methylthio)acetophenone to obtain a white solid, yield was 39%; ESA-MS: m/z=388.1 [M+H]⁺.

### 5-(3-(Diethylamino)phenyl)-2-methyl-N-(3-(2-oxopropyl)-1,2,4-thiadiazol-5-yl)furan-3-carboxamide (Compound A-22)

Preparation of compound A-22 referred to the synthesis of compound A-1, replacing m-trifluoromethyl acetophenone with 3-(diethylamino)acetophenone to obtain a light yellow solid, yield was 81%; ESA-MS: m/z=413.2 [M+H]⁺.

### 5-(3-(Dimethylamino)phenyl)-2-methyl-N-(3-(2-oxopropyl)-1,2,4-thiadiazol-5-yl)furan-3-carboxamide (Compound A-23)

Preparation of compound A-23 referred to the synthesis of compound A-1, replacing m-trifluoromethyl acetophenone with 3-(dimethylamino)acetophenone to obtain a light yellow solid, yield was 85%; ESA-MS: m/z=385.1 [M+H]⁺.

### Example 2: Synthesis of Compounds A-24˜A-30

The synthetic route of compounds A-24˜A-30 is shown in FIG. 3.

### 5-(3-Aminophenyl)-2-methyl-N-(3-(2-oxopropyl)-1,2,4-thiadiazol-5-yl)furan-3-carboxamide (Compound A-24)

To a solution of compound A-2 (215 mg, 0.56 mmol) in ethanol and water (3:1, 12 mL) was added iron powder (225 mg, 4.0 mmol) and ammonium chloride (89 mg, 1.7 mmol), and the mixture was stirred at 75° C. for 6 hours. The reaction mixture was adjusted with saturated sodium bicarbonate solution to weak alkalinity, then ethyl acetate was added. The organic layer was washed with water and brine, dried over anhydrous Na₂SO₄, and concentrated to give a residue, which was purified by column-chromatograph to give a light yellow solid, yield was 72%; ¹H NMR (500 MHZ, DMSO-d6): δ 13.12 (s, 1H), 7.54 (s, 1H), 7.10 (t, J=8.0 Hz, 1H), 6.87 (t, J=2.0 Hz, 1H), 6.80-6.76 (m, 1H), 6.57-6.52 (m, 1H), 5.30 (s, 2H), 4.04 (s, 2H), 2.68 (s, 3H), 2.21 (s, 3H); ESA-MS: m/z=357.1 [M+H]⁺.

### 5-(3-Acetamidophenyl)-2-methyl-N-(3-(2-oxopropyl)-1,2,4-thiadiazol-5-yl)furan-3-carboxamide (Compound A-25)

To a solution of compound A-24 (30 mg, 0.08 mmol) and triethylamine (100 μL. 0.80 mmol) in ethanol and acetonitrile (1:1, 10 mL) was added acetyl chloride (30 μL, 0.42 mmol) dropwise under ice bath, and then the reaction was heated to reflux for 4 hours. Ethyl acetate was added to the reaction mixture and the organic layer was washed with water and brine, dried over anhydrous Na₂SO₄, and concentrated to give a residue, which was purified by column-chromatograph to give a light yellow solid, yield was 82%; ¹H NMR (500 MHz, DMSO-d6): δ 13.18 (s, 1H), 10.11 (s, 1H), 8.07 (t, J=2.0 Hz, 1H), 7.68 (s, 1H), 7.48-7.44 (m, 1H), 7.39 (t, J=8.0 Hz, 1H), 7.32-7.28 (m, 1H), 4.04 (s, 2H), 2.71 (s, 3H), 2.21 (s, 3H), 2.08 (s, 3H); ESA-MS: m/z=399.1 [M+H]⁺.

### 5-(3-Hydroxyphenyl)-2-methyl-N-(3-(2-oxopropyl)-1,2,4-thiadiazol-5-yl)furan-3-carboxamide (Compound A-26)

To a solution of compound A-4 (100 mg, 0.27 mmol) in anhydrous dichloromethane (6 mL) was slowly added boron tribromide (2.0 mol/L in dichloromethane, 404 μL, 0.81 mmol) at −78° C. under nitrogen atmosphere. The reaction was then moved to room temperature and stirred overnight and quenched with water. Ethyl acetate was added to the reaction mixture, and the organic layer was washed with water and brine, dried over anhydrous Na₂SO₄, and concentrated to give a residue, which was purified by column-chromatograph to give a white solid, yield was 73%; ¹H NMR (500 MHZ, DMSO-d6): δ 13.13 (s, 1H), 9.69 (s, 1H), 7.61 (s, 1H), 7.27 (t, J=8.0 Hz, 1H), 7.09-7.02 (m, 2H), 6.77-7.73 (m, 1H), 4.04 (s, 2H), 2.69 (s, 3H), 2.20 (s, 3H); ESA-MS: m/z=358.1 [M+H]⁺.

### 3-(5-Methyl-4-((3-(2-oxopropyl)-1,2,4-thiadiazol-5-yl) carbamoyl)furan-2-yl)phenyl acetate (Compound A-27)

To a solution of compound A-26 (50 mg, 0.14 mmol) in acetonitrile (5 mL) was added triethylamine (20 μL. 0.14 mmol) and acetyl chloride (20 μL, 0.28 mmol) at 0° C. The solution was moved to room temperature and stirred for 6 hours. Ethyl acetate was added to the reaction mixture, and the organic layer was washed with water and brine, dried over anhydrous Na₂SO₄, and concentrated to give a residue, which was purified by column-chromatograph to give a white solid, yield was 62%; ¹H NMR (500 MHZ, DMSO-d₆): δ 13.15 (s, 1H), 7.72 (s, 1H), 7.55-7.49 (m, 2H), 7.41-7.38 (m, 1H), 7.14-7.10 (m, 1H), 4.04 (s, 2H), 2.70 (s, 3H), 2.31 (s, 3H), 2.21 (s, 3H); ESI-MS: m/z=400.1 [M+H]⁺.

### 5-(3-Carbamoylphenyl)-2-methyl-N-(3-(2-oxopropyl)-1,2,4-thiadiazol-5-yl)furan-3-carboxamide (Compound A-28)

To a solution of compound A-3 (1.84 g, 5.02 mmol) and 30% H₂O₂ (1.8 mL, 17.5 mmol) in ethanol (5 mL) was added 6 mol/L sodium hydroxide solution (0.21 mL, 1.26 mmol). The reaction mixture was stirred at 50° C. overnight and neutralized with 6 mol/L HCl. Dichloromethane was added to the reaction mixture, and the organic layer was washed with water and brine, dried over anhydrous Na₂SO₄, and concentrated to give a residue, which was purified by column-chromatograph to give a white solid, yield was 88%; ¹H NMR (500 MHz, DMSO-d₆): δ 13.16 (s, 1H), 8.13 (s, 1H), 8.17 (t, J=1.5 Hz, 1H), 7.86-7.82 (m, 1H), 7.78-7.77 (m, 2H), 7.56 (t, J=8.0 Hz, 1H), 7.50 (s, 1H), 4.04 (s, 2H), 2.73 (s, 3H), 2.21 (s, 3H); ESI-MS: m/z=385.1 [M+H]⁺.

### 3-(5-Methyl-4-((3-(2-oxopropyl)-1,2,4-thiadiazol-5-yl) carbamoyl)furan-2-yl)benzoic acid (Compound A-29)

A solution of compound A-28 (500 mg, 1.30 mmol) in concentrated sulfuric acid and water (9 mL, 1:3) was stirred at 100° C. for 5 hours. Ethyl acetate was added to the reaction mixture, and the organic layer was washed with water and brine, dried over anhydrous Na₂SO₄, and concentrated to give a residue, which was purified by column-chromatograph to give a white solid, yield was 32%; ¹H NMR (500 MHZ, Chloroform-d): δ 8.19 (t. J=1.5 Hz, 1H), 8.06-8.02 (m, 1H), 7.92-7.88 (m, 1H), 7.56 (t, J=7.5 Hz, 1H), 7.20 (s, 1H), 3.56 (s, 2H), 2.59 (s, 3H), 2.28 (s, 3H); ESA-MS: m/z=386.1 [M+H]⁺.

### Methyl 3-(5-methyl-4-((3-(2-oxopropyl)-1,2,4-thiadiazol-5-yl) carbamoyl)furan-2-yl)benzoate (Compound A-30)

A solution of compound A-29 (120 mg, 0.31 mmol), 4-dimethylaminopyridine (4 mg, 0.03 mmol) and methanol (38 μL, 0.93 mmol) in dichloromethane (6 mL) was stirred at room temperature for 30 min and then cooled to 0° C. After adding dicyclohexylcarbodiimide (64 mg, 0.31 mmol), the reaction mixture was stirred at room temperature overnight. Ethyl acetate was added to the reaction mixture, and the organic layer was washed with water and brine, dried over anhydrous Na₂SO₄, and concentrated to give a residue, which was purified by column-chromatograph to give a white solid, yield was 32%; ¹H NMR (500 MHZ, Chloroform-d): δ 8.26 (t, J=1.5 Hz, 1H), 7.98-7.94 (m, 1H), 7.88-7.84 (m, 1H), 7.57 (t, J=7.5 Hz, 1H), 7.04 (s, 1H), 3.93 (s, 3H), 3.58 (s, 2H), 2.61 (s, 3H), 2.28 (s, 3H); ESA-MS: m/z=400.1 [M+H]⁺.

### Example 3: Synthesis of Compounds A-31˜A-33

### 2-Methyl-N-(3-(2-oxopropyl)-1,2,4-thiadiazol-5-yl)-5-(pyridin-3-yl)furan-3-carboxamide (Compound A-31)

The synthesis route is shown in FIG. 4.

### (a) Synthesis of methyl 2-methyl-5-(pyridin-3-yl)furan-3-carboxylate (Compound II-2-1)

A suspension of 3-bromopyridine (II-1-1, 122 μL, 1.27 mmol), methyl 2-methylfuran-3-carboxylate (317 μL, 2.53 mmol), potassium acetate (248 mg, 2.53 mmol) and palladium acetate (0.1% equivalent) in DMA (5 mL) was stirred at 150° C. overnight. Ethyl acetate was added to the reaction mixture, and the organic layer was washed with water and brine, dried over anhydrous Na₂SO₄, and concentrated to give a residue, which was purified by column-chromatograph to give a yellow solid, yield was 88%; ESA-MS: m/z=218.1 [M+H]⁺.

### (b) Synthesis of 2-methyl-5-(pyridin-3-yl)furan-3-carboxylic acid (Compound II-3-1)

A solution of compound II-2-1 (140 mg, 0.64 mmol) and lithium hydroxide (48 mg, 2.00 mmol) in methanol and water (3:1, 4 mL) was heated to reflux for 1 hour. The reaction mixture was adjusted with 2.0 mol/L HCl to acidity, then ethyl acetate was added. The organic layer was washed with water and brine, dried over anhydrous Na₂SO₄, and concentrated to give a residue, which was purified by column-chromatograph to give a white solid, yield was 81%; ESA-MS: m/z=204.1 [M+H]⁺.

### (c) Synthesis of 2-methyl-5-(pyridin-3-yl)furan-3-carbonyl chloride (Compound II-4-1)

A solution of compound II-3-1 (73 mg, 0.36 mmol) in thionyl chloride (10 mL) was refluxed for 2.5 hours. The reaction mixture was concentrated under reduced pressure to obtain a light brown solid, yield was 95%;

### (d) Synthesis of 2-methyl-N-(3-(2-oxopropyl)-1,2,4-thiadiazol-5-yl)-5-(pyridin-3-yl)furan-3-carboxamide (Compound A-31)

A suspension of compound II-4-1 (77 mg, 0.34 mmol), sodium bicarbonate (88 mg, 1.04 mmol) and potassium thiocyanate (37 mg, 0.38 mmol) in acetonitrile (15 mL) was stirred at 60° C. for 30 min, and then 3-amino-5-methylisoxazole (34 mg, 0.35 mmol) was added and reacted for 1 hour. Ethyl acetate was added and the organic layer was washed with water and brine, dried over anhydrous Na₂SO₄, and concentrated to give a residue, which was purified by column-chromatograph to give a yellow solid, yield was 81%; ¹HNMR (500 MHz, DMSO-d₆): δ 13.20 (s, 1H), 8.86 (s, 1H), 8.55 (d, J=4.0 Hz, 1H), 7.98 (d, J=8.0 Hz, 1H), 7.78 (s, 1H), 7.51 (dd, J₁=8.0 Hz, J₂=4.0 Hz, 1H), 4.04 (s, 2H), 2.71 (s, 3H), 2.21 (s, 3H); ESA-MS: m/z=343.1 [M+H]⁺.

### 2-Methyl-N-(3-(2-oxopropyl)-1,2,4-thiadiazol-5-yl)-5-(pyridin-2-yl)furan-3-carboxamide (Compound A-32)

Preparation of compound A-32 referred to the synthesis of compound A-31, replacing 3-bromopyridine with 2-bromopyridine to obtain a light yellow solid, yield was 57%; ESA-MS: m/z=343.1 [M+H]⁺.

### 2-Methyl-N-(3-(2-oxopropyl)-1,2,4-thiadiazol-5-yl)-5-(pyridin-4-yl)furan-3-carboxamide (Compound A-33)

Preparation of compound A-33 referred to the synthesis of compound A-31, replacing 3-bromopyridine with 4-bromopyridine to obtain a light yellow solid, yield was 50%; ESA-MS: m/z=343.1 [M+H]⁺.

### Example 4: Synthesis of Compounds A-34˜A-37

The synthesis route is shown in FIG. 5.

### 2-Methyl-N-(3-(2-oxopropyl)-1,2,4-thiadiazol-5-yl)-5-(3-(trifluoromethyl)phenyl)furan-4-d-3-carboxamide (Compound A-34)

The specific synthesis route is shown in FIG. 6.

Compound III-1-1/I-1-1 was deuterated with deuterium oxide, brominated by N-bromosuccinimide and then reacted with ethyl acetoacetate, cyclized under the action of trifluoromethanesulfonic acid, hydrolyzed and reacted with thionyl chloride to form acid chloride, and then reacted with potassium thiocyanate and 3-amino-5-methylisoxazole to obtain the target compound A-34. White solid, yield was 32%; ¹H NMR (500 MHz, Chloroform-d): δ 8.02 (t, J=1.5 Hz, 1H), 7.70-7.61 (m, 2H), 7.59-7.53 (m, 1H), 3.56 (s, 2H), 2.53 (s, 3H), 2.28 (s, 3H); ESA-MS: m/z=412.1 [M+H]⁺.

### 5-(3-Cyanophenyl)-2-methyl-N-(3-(2-oxopropyl)-1,2,4-thiadiazol-5-yl)furan-4-d-3-carboxamide (Compound A-35)

Preparation of compound A-35 referred to the synthesis of compound A-34, replacing 3-trifluoromethylacetophenone with 3-cyanoacetophenone to obtain a light yellow solid, yield was 35%; ESA-MS: m/z=368.1 [M+H]⁺.

### 2-Methyl-N-(3-(2-oxopropyl)-1,2,4-thiadiazol-5-yl)-5-(3-(trifluoromethoxy)phenyl)furan-4-d-3-carboxamide (Compound A-36)

Preparation of compound A-36 referred to the synthesis of compound A-34, replacing 3-trifluoromethylacetophenone with 3-trifluoromethoxyacetophenone to obtain a white solid, yield was 31%; ESA-MS: m/z=427.1 [M+H]⁺.

### 5-(3-(Difluoromethoxy)phenyl)-2-methyl-N-(3-(2-oxopropyl)-1,2,4-thiadiazol-5-yl)furan-4-d-3-carboxamide (Compound A-37)

Preparation of compound A-37 referred to the synthesis of compound A-34, replacing 3-trifluoromethylacetophenone with 3-difluoromethoxyacetophenone to obtain a light yellow solid, yield was 32%; ESA-MS: m/z=409.1 [M+H]⁺.

### Example 5: Synthesis of Compounds A-38˜A-43

The synthesis route is shown in FIG. 7.

### N-(3-(2-(dimethylamino)propyl)-1,2,4-thiadiazol-5-yl)-2-methyl-5-(3-(trifluoromethyl)phenyl)furan-3-carboxamide (Compound A-38)

The specific synthesis route is shown in FIG. 8.

To a solution of compound A-1 (100 mg, 0.24 mmol) in anhydrous ethanol (5 mL) was added triethylamine (49 mg, 0.49 mmol), titanium tetraisopropoxide (139 mg, 0.49 mmol) and dimethylamine hydrochloride (40 mg, 0.49 mmol), and the reaction mixture was stirred at room temperature for 48 hours. Sodium borohydride (14 mg, 0.37 mmol) was added and stirred for another 12 hours. The reaction was quenched with saturated ammonium chloride solution and alkalized with saturated sodium bicarbonate solution. Ethyl acetate was added and the organic layer was washed with water and brine, dried over anhydrous Na₂SO₄, and concentrated to give a residue, which was purified by column-chromatograph to give a light yellow solid, yield was 72%; ¹H NMR (500 MHZ, Chloroform-d): δ 8.08 (t, J=1.5 Hz, 1H), 7.80-7.72 (m, 1H), 7.71-7.68 (m, 1H), 7.60 (t, J=7.5 Hz, 1H), 6.99 (s, 1H), 3.10 (m, 1H), 2.80 (dd, J₁=12.5 Hz, J₂=7.0 Hz, 1H), 2.70 (dd, J₁=12.5 Hz, J₂=7.0 Hz, 1H), 2.53 (s, 3H), 2.46 (s, 6H), 1.11 (d, J=7.0 Hz, 3H); ESA-MS: m/z=439.1 [M+H]⁺.

### N-(3-(2-(diethylamino)propyl)-1,2,4-thiadiazol-5-yl)-2-methyl-5-(3-(trifluoromethyl)phenyl)furan-3-carboxamide (Compound A-39)

Preparation of compound A-39 referred to the synthesis of compound A-38, replacing dimethylamine hydrochloride with diethylamine hydrochloride to obtain a white solid, yield was 75%; ESA-MS: m/z=467.2 [M+H]⁺.

### 2-Methyl-N-(3-(2-(pyrrolidin-1-yl)propyl)-1,2,4-thiadiazol-5-yl)-5-(3-(trifluoromethyl)phenyl)furan-3-carboxamide (Compound A-40)

Preparation of compound A-40 referred to the synthesis of compound A-38, replacing dimethylamine hydrochloride with pyrrolidine to obtain a light yellow solid, yield was 74%; ESA-MS: m/z=465.2 [M+H]⁺.

### 2-Methyl-N-(3-(2-(piperidin-1-yl)propyl)-1,2,4-thiadiazol-5-yl)-5-(3-(trifluoromethyl) phenyl)furan-3-carboxamide (Compound A-41)

Preparation of compound A-41 referred to the synthesis of compound A-38, replacing dimethylamine hydrochloride with piperidine to obtain a white solid, yield was 76%; ESA-MS: m/z=479.2 [M+H]⁺.

### 2-Methyl-N-(3-(2-morpholinopropyl)-1,2,4-thiadiazol-5-yl)-5-(3-(trifluoromethyl)phenyl) furan-3-carboxamide (Compound A-42)

Preparation of compound A-42 referred to the synthesis of compound A-38, replacing dimethylamine hydrochloride with morpholine to obtain a white solid, yield was 73%; ESA-MS: m/z=481.1 [M+H]⁺.

### 2-Methyl-N-(3-(2-(4-methylpiperazin-1-yl)propyl)-1,2,4-thiadiazol-5-yl)-5-(3-(trifluoro-methyl)phenyl)furan-3-carboxamide (Compound A-43)

Preparation of compound A-43 referred to the synthesis of compound A-38, replacing dimethylamine hydrochloride with N-methylpiperazine to obtain a white solid, yield was 70%; ESA-MS: m/z=494.2 [M+H]⁺.

### Example 6: Synthesis of Compounds A-44˜A-59

### N-(3-(2-(dimethylamino)propyl)-1,2,4-thiadiazol-5-yl)-2-methyl-5-(3-(trifluoromethoxy)phenyl)furan-3-carboxamide (Compound A-44)

Preparation of compound A-44 referred to the synthesis of compound A-38, using compound A-13 as the starting material to obtain a white solid, yield was 59%; ESA-MS: m/z=455.1 [M+H]⁺.

### N-(3-(2-(diethylamino)propyl)-1,2,4-thiadiazol-5-yl)-5-(3-(difluoromethoxy)phenyl)-2-methylfuran-3-carboxamide (Compound A-45)

Preparation of compound A-45 referred to the synthesis of compound A-39, using compound A-14 as the starting material to obtain a white solid, yield was 63%; ESA-MS: m/z=465.2 [M+H]⁺.

### 5-(3-Cyanophenyl)-2-methyl-N-(3-(2-(pyrrolidin-1-yl)propyl)-1,2,4-thiadiazol-5-yl)furan-3-carboxamide (Compound A-46)

Preparation of compound A-46 referred to the synthesis of compound A-40, using compound A-3 as the starting material to obtain a white solid, yield was 68%; ESA-MS: m/z=422.2 [M+H]⁺.

### 5-(3-Methoxyphenyl)-2-methyl-N-(3-(2-(pyrrolidin-1-yl)propyl)-1,2,4-thiadiazol-5-yl)furan-3-carboxamide (Compound A-47)

Preparation of compound A-47 referred to the synthesis of compound A-40, using compound A-4 as the starting material to obtain a white solid, yield was 64%; ESA-MS: m/z=427.2 [M+H]⁺.

### 2-Methyl-N-(3-(2-(pyrrolidin-1-yl)propyl)-1,2,4-thiadiazol-5-yl)-5-(3-(trifluoromethoxy)phenyl)furan-3-carboxamide (Compound A-48)

Preparation of compound A-48 referred to the synthesis of compound A-40, using compound A-13 as the starting material to obtain a white solid, yield was 67%; ESA-MS: m/z=481.1 [M+H]⁺.

### 5-(3-Methoxyphenyl)-2-methyl-N-(3-(2-(piperidin-1-yl)propyl)-1,2,4-thiadiazol-5-yl)furan-3-carboxamide (Compound A-49)

Preparation of compound A-49 referred to the synthesis of compound A-41, using compound A-4 as the starting material to obtain a white solid, yield was 67%; ESA-MS: m/z=441.2 [M+H]⁺.

### 2-Methyl-N-(3-(2-(piperidin-1-yl)propyl)-1,2,4-thiadiazol-5-yl)-5-(3-(trifluoromethoxy)phenyl)furan-3-carboxamide (Compound A-50)

Preparation of compound A-50 referred to the synthesis of compound A-41, using compound A-13 as the starting material to obtain a white solid, yield was 62%; ESA-MS: m/z=495.2 [M+H]⁺.

### 5-(3-(Difluoromethoxy)phenyl)-2-methyl-N-(3-(2-(piperidin-1-yl)propyl)-1,2,4-thiadiazol-5-yl)furan-3-carboxamide (Compound A-51)

Preparation of compound A-51 referred to the synthesis of compound A-41, using compound A-14 as the starting material to obtain a white solid, yield was 61%; ESA-MS: m/z=477.2 [M+H]⁺. 5-(3-Cyanophenyl)-2-methyl-N-(3-(2-morpholinopropyl)-1,2,4-thiadiazol-5-yl)furan-3-carboxamide (Compound A-52)

Preparation of compound A-52 referred to the synthesis of compound A-42, using compound A-3 as the starting material to obtain a white solid, yield was 63%; ESA-MS: m/z=438.2 [M+H]⁺. 5-(3-Methoxyphenyl)-2-methyl-N-(3-(2-morpholinopropyl)-1,2,4-thiadiazol-5-yl)furan-3-carboxamide (Compound A-53)

Preparation of compound A-53 referred to the synthesis of compound A-42, using compound A-4 as the starting material to obtain a white solid, yield was 57%; ESA-MS: m/z=443.2 [M+H]⁺.

### 2-Methyl-N-(3-(2-morpholinopropyl)-1,2,4-thiadiazol-5-yl)-5-(3-(trifluoromethoxy)phenyl)furan-3-carboxamide (Compound A-54)

Preparation of compound A-54 referred to the synthesis of compound A-42, using compound A-13 as the starting material to obtain a white solid, yield was 68%; ESA-MS: m/z=497.1 [M+H]⁺.

### 5-(3-(Difluoromethoxy)phenyl)-2-methyl-N-(3-(2-morpholinopropyl)-1,2,4-thiadiazol-5-yl)furan-3-carboxamide (Compound A-55)

Preparation of compound A-55 referred to the synthesis of compound A-42, using compound A-14 as the starting material to obtain a white solid, yield was 60%; ESA-MS: m/z=479.2 [M+H]⁺.

### 5-(3-Cyanophenyl)-2-methyl-N-(3-(2-(4-methylpiperazin-1-yl)propyl)-1,2,4-thiadiazol-5-yl)furan-3-carboxamide (Compound A-56)

Preparation of compound A-56 referred to the synthesis of compound A-43, using compound A-3 as the starting material to obtain a white solid, yield was 54%; ESA-MS: m/z=451.2 [M+H]⁺.

### 5-(3-Methoxyphenyl)-2-methyl-N-(3-(2-(4-methylpiperazin-1-yl)propyl)-1,2,4-thiadiazol-5-yl)furan-3-carboxamide (Compound A-57)

Preparation of compound A-57 referred to the synthesis of compound A-43, using compound A-4 as the starting material to obtain a white solid, yield was 64%; ESA-MS: m/z=456.2 [M+H]⁺.

### 2-Methyl-N-(3-(2-(4-methylpiperazin-1-yl)propyl)-1,2,4-thiadiazol-5-yl)-5-(3-(trifluoro-methoxy)phenyl) furan-3-carboxamide (Compound A-58)

Preparation of compound A-58 referred to the synthesis of compound A-43, using compound A-13 as the starting material to obtain a white solid, yield was 61%; ESA-MS: m/z=510.2 [M+H]⁺.

### 5-(3-(Difluoromethoxy)phenyl)-2-methyl-N-(3-(2-(4-methylpiperazin-1-yl)propyl)-1,2,4-thiadiazol-5-yl)furan-3-carboxamide (Compound A-59)

Preparation of compound A-59 referred to the synthesis of compound A-43, using compound A-14 as the starting material to obtain a white solid, yield was 57%; ESA-MS: m/z=492.2 [M+H]⁺.

### Example 7: Synthesis of Compounds A-60˜A-65

### N-(3-(2-hydroxypropyl)-1,2,4-thiadiazol-5-yl)-2-methyl-5-(3-(trifluoromethyl)phenyl)furan-3-carboxamide (Compound A-60)

The synthesis route is shown in FIG. 9.

To a solution of compound A-1 (200 mg, 0.49 mmol) in methanol (5 mL) was added sodium borohydride (28 mg, 0.73 mmol) and reacted at room temperature for 3 hours. The reaction was quenched with saturated ammonium chloride solution, and then ethyl acetate was added. The organic layer was washed with water and brine, dried over anhydrous Na₂SO₄, and concentrated to give a residue, which was purified by column-chromatograph to give a light yellow solid, yield was 80%; ¹H NMR (500 MHZ, Chloroform-d): δ 8.09 (t, J=1.5 Hz, 1H), 7.82-7.78 (m, 1H), 7.69-7.65 (m, 1H), 7.62 (t, J=7.5 Hz, 1H), 7.00 (s, 1H), 4.40 (d, J=8.5 Hz, 1H), 4.18-4.13 (m, 1H), 2.76 (dd, J₁=12.5 Hz, J₂=7.0 Hz, 1H), 2.68 (dd, J₁=12.5 Hz, J₂=7.0 Hz, 1H), 2.63 (s, 3H), 1.29 (d, J=6.8 Hz, 3H); ESA-MS: m/z=412.1 [M+H]⁺.

### N-(3-(2-methoxypropyl)-1,2,4-thiadiazol-5-yl)-2-methyl-5-(3-(trifluoromethyl)phenyl)furan-3-carboxamide (Compound A-61)

A solution of compound A-60 (100 mg, 0.24 mmol) and sodium hydroxide (15 mg, 0.36 mmol) in anhydrous DMF (4 mL) was stirred for 30 min, and then methyl iodide (41 mg, 0.29 mmol) was added and reacted for 1.5 hours. Ethyl acetate was added and the organic layer was washed with water and brine, dried over anhydrous Na₂SO₄, and concentrated to give a residue, which was purified by column-chromatograph to give a light yellow solid, yield was 61%; ¹H NMR (500 MHZ, Chloroform-d): δ 8.08 (t, J=1.5 Hz, 1H), 7.80-7.76 (m, 1H), 7.69-7.65 (m, 1H), 7.62 (t, J=7.5 Hz, 1H), 6.99 (s, 1H), 4.13 (q, J=7.0 Hz, 1H), 3.29 (s, 3H), 2.98-2.71 (m, 2H), 2.60 (s, 3H), 1.45 (d, J=7.0 Hz, 3H); ESA-MS: m/z=426.1 [M+H]⁺.

### N-(3-(2-hydroxypropyl)-1,2,4-thiadiazol-5-yl)-2-methyl-5-(3-(trifluoromethoxy)phenyl) furan-3-carboxamide (Compound A-62)

Preparation of compound A-62 referred to the synthesis of compound A-60, using compound A-13 as the starting material to obtain a light yellow solid, yield was 78%; ESA-MS: m/z=428.1 [M+H]⁺.

### N-(3-(2-methoxypropyl)-1,2,4-thiadiazol-5-yl)-2-methyl-5-(3-(trifluoromethoxy)phenyl) furan-3-carboxamide (Compound A-63)

Preparation of compound A-63 referred to the synthesis of compound A-61, using compound A-62 as the starting material to obtain a light yellow solid, yield was 65%; ESA-MS: m/z=442.1 [M+H]⁺.

### 5-(3-(Difluoromethoxy)phenyl)-N-(3-(2-hydroxypropyl)-1,2,4-thiadiazol-5-yl)-2-methyl-furan-3-carboxamide (Compound A-64)

Preparation of compound A-64 referred to the synthesis of compound A-60, using compound A-14 as the starting material to obtain a light yellow solid, yield was 75%; ESA-MS: m/z=410.1 [M+H]⁺.

### 5-(3-(Difluoromethoxy)phenyl)-N-(3-(2-methoxypropyl)-1,2,4-thiadiazol-5-yl)-2-methyl-furan-3-carboxamide (Compound A-65)

Preparation of compound A-65 referred to the synthesis of compound A-61, using compound A-64 as the starting material to obtain a light yellow solid, yield was 55%; ESA-MS: m/z=424.1 [M+H]⁺.

### Example 8: Synthesis of Compounds A-66˜A-68

### N-(3-(2-aminopropyl)-1,2,4-thiadiazol-5-yl)-2-methyl-5-(3-(trifluoromethyl)phenyl)furan-3-carboxamide (Compound A-66)

The synthesis route is shown in FIG. 10.

### (a) Synthesis of 1-(5-(2-methyl-5-(3-(trifluoromethyl)phenyl)furan-3-carboxamido)-1,2,4-thiadiazol-3-yl) propan-2-yl 4-methylbenzenesulfonate (Compound IV-2-1)

A solution of compound A-60/IV-1-1 (200 mg, 0.49 mmol), p-toluenesulfonyl chloride (102 mg, 0.53 mmol) and N,N-diisopropylethylamine (102 μL, 0.58 mmol) in acetonitrile (10 mL) was stirred at room temperature overnight. Ethyl acetate was added to the reaction mixture and the organic layer was washed with water and brine, dried over anhydrous Na₂SO₄, and concentrated to give a residue, which was purified by column-chromatograph to give a light yellow solid, yield was 82%; ESA-MS: m/z=566.1 [M+H]⁺.

### (b) Synthesis of N-(3-(2-azidopropyl)-1,2,4-thiadiazol-5-yl)-2-methyl-5-(3-(trifluoromethyl)phenyl)furan-3-carboxamide (Compound IV-3-1)

A solution of compound IV-2-1 (220 mg, 0.39 mmol) and sodium azide (379 mg, 5.8 mmol) in anhydrous DMF (5 mL) was stirred under nitrogen atmosphere for 15 hours. Ethyl acetate was added to the reaction mixture and the organic layer was washed with water and brine, dried over anhydrous Na₂SO₄, and concentrated to give a residue, which was purified by column-chromatograph to give a light yellow solid, yield was 85%; ESA-MS: m/z=437.1 [M+H]⁺.

### (c) Synthesis of N-(3-(2-aminopropyl)-1,2,4-thiadiazol-5-yl)-2-methyl-5-(3-(trifluoromethyl)phenyl)furan-3-carboxamide (Compound A-66)

A solution of triphenylphosphine (95 mg, 0.36 mmol) and compound IV-3-1 (144 mg, 0.33 mmol) in THF and water (10:1, 5.5 mL) was stirred at room temperature overnight. Ethyl acetate was added to the reaction mixture and the organic layer was washed with water and brine, dried over anhydrous Na₂SO₄, and concentrated to give a residue, which was purified by column-chromatograph to give a light yellow solid, yield was 72%; ¹H NMR (500 MHZ, Chloroform-d) δ 8.09 (t, J=1.5 Hz, 1H), 7.84-7.79 (m, 1H), 7.70-7.66 (m, 1H), 7.62 (t, J=7.5 Hz, 1H), 7.05 (s, 1H), 4.92 (dd, J₁=8.5 Hz, J₂=7.0 Hz, 1H), 4.74 (dd, J₁=8.5 Hz, J₂=7.0 Hz, 1H), 3.54-3.49 (m, 1H), 2.79-2.64 (m, 2H), 2.62 (s, 3H), 1.26 (d, J=7.0 Hz, 3H); ESA-MS: m/z=411.1 [M+H]⁺.

### N-(3-(2-aminopropyl)-1,2,4-thiadiazol-5-yl)-2-methyl-5-(3-(trifluoromethoxy)phenyl)furan-3-carboxamide (Compound A-67)

Preparation of compound A-67 referred to the synthesis of compound A-66, using compound A-62 as the starting material to obtain a white solid, yield was 48%; ESA-MS: m/z=427.1 [M+H]⁺.

### N-(3-(2-aminopropyl)-1,2,4-thiadiazol-5-yl)-5-(3-(difluoromethoxy)phenyl)-2-methylfuran-3-carboxamide (Compound A-68)

Preparation of compound A-68 referred to the synthesis of compound A-66, using compound A-64 as the starting material to obtain a white solid, yield was 42%; ESA-MS: m/z=409.1 [M+H]⁺.

### Example 9: Synthesis of Compounds A-69˜A-74

### N-(3-(2-(hydroxyimino)propyl)-1,2,4-thiadiazol-5-yl)-2-methyl-5-(3-(trifluoromethyl)phenyl)furan-3-carboxamide (Compound A-69)

The synthesis route is shown in FIG. 11.

A solution of compound A-1 (100 mg, 0.24 mmol), hydroxylamine hydrochloride (25 mg, 0.36 mmol) and sodium acetate (30 mg, 0.36 mmol) in methanol and water (5 mL, 20:1) was stirred at room temperature for 4 hours. Ethyl acetate was added to the reaction mixture and the organic layer was washed with water, sodium bicarbonate solution and brine, dried over anhydrous Na₂SO₄, and concentrated to give a residue, which was purified by column-chromatograph to give a white solid, yield was 75%; ¹H NMR (500 MHZ, Chloroform-d): δ 9.52 (s, 1H), 8.09 (t, J=1.5 Hz, 1H), 7.79-7.75 (m, 1H), 7.71-7.59 (m, 2H), 7.00 (s, 1H), 3.14 (s. 2H), 2.60 (s, 3H), 2.19 (s, 3H); ESA-MS: m/z=425.1 [M+H]⁺.

### N-(3-(2-(methoxyimino)propyl)-1,2,4-thiadiazol-5-yl)-2-methyl-5-(3-(trifluoromethyl)phenyl)furan-3-carboxamide (Compound A-70)

To a solution of compound A-69 (100 mg, 0.24 mmol) and potassium carbonate (37 mg, 0.27 mmol) in acetone (5 mL) was added methyl iodide (69 mg, 0.49 mmol) dropwise, and stirred at 40° C. for 4 hours. Ethyl acetate was added to the reaction mixture and the organic layer was washed with water and brine, dried over anhydrous Na₂SO₄, and concentrated to give a residue, which was purified by column-chromatograph to give a light yellow solid, yield was 55%; ¹H NMR (500 MHZ, Chloroform-d): δ 8.09 (t, J=1.5 Hz, 1H), 7.80-7.76 (m. 1H), 7.72-7.68 (m, 1H), 7.61 (t, J=7.5 Hz, 1H), 6.99 (s, 1H), 3.96 (s, 3H), 3.15 (s. 2H), 2.54 (s, 3H), 2.32 (s. 3H); ESA-MS: m/z=439.1 [M+H]⁺.

### 5-(3-Cyanophenyl)-N-(3-(2-(hydroxyimino)propyl)-1,2,4-thiadiazol-5-yl)-2-methylfuran-3-carboxamide (Compound A-71)

Preparation of compound A-71 referred to the synthesis of compound A-69, using compound A-3 as the starting material to obtain a white solid, yield was 71%; ESA-MS: m/z=382.1 [M+H]⁺.

### 5-(3-Cyanophenyl)-N-(3-(2-(methoxyimino)propyl)-1,2,4-thiadiazol-5-yl)-2-methylfuran-3-carboxamide (Compound A-72)

Preparation of compound A-72 referred to the synthesis of compound A-70, using compound A-71 as the starting material to obtain a white solid, yield was 73%; ESA-MS: m/z=396.1 [M+H]⁺.

### 5-(3-(Difluoromethoxy)phenyl)-N-(3-(2-(hydroxyimino)propyl)-1,2,4-thiadiazol-5-yl)-2-methylfuran-3-carboxamide (Compound A-73)

Preparation of compound A-73 referred to the synthesis of compound A-69, using compound A-14 as the starting material to obtain a white solid, yield was 73%; ESA-MS: m/z=423.1 [M+H]⁺.

### 5-(3-(Difluoromethoxy)phenyl)-N-(3-(2-(methoxyimino)propyl)-1,2,4-thiadiazol-5-yl)-2-methylfuran-3-carboxamide (Compound A-74)

Preparation of compound A-74 referred to the synthesis of compound A-70, using compound A-73 as the starting material to obtain a white solid, yield was 73%; ESA-MS: m/z=437.1 [M+H]⁺.

### Example 10: Synthesis of Compounds A-75˜A-79

### 2-Methyl-N-(3-(3,3,3-trifluoro-2-hydroxy-2-methylpropyl)-1,2,4-thiadiazol-5-yl)-5-(3-(trifluoromethyl)phenyl)furan-3-carboxamide (Compound A-75)

The synthesis route is shown in FIG. 12.

To a solution of compound A-1 (100 mg, 0.24 mmol) and TMSCF₃ (42 mg, 0.29 mmol) in anhydrous THF (5 mL) was added TBAF (0.6 mg, 0.002 mmol) at 0° C. and the mixture was then moved to room temperature and react for 2 hours. An appropriate amount of 1 mol/L HCl was added and stirred for 30 min. Ethyl acetate was added to the reaction mixture and the organic layer was washed with water and brine, dried over anhydrous Na₂SO₄, and concentrated to give a residue, which was purified by column-chromatograph to give a light yellow solid, yield was 72%; ¹H NMR (500 MHZ, Chloroform-d): δ 8.10 (t, J=1.5 Hz, 1H), 7.79-7.75 (m, 1H), 7.73-7.69 (m, 1H), 7.60 (t, J=7.5 Hz, 1H), 6.99 (s, 1H), 4.55 (s, 1H), 2.87 (s. 2H), 2.56 (s, 3H), 1.42 (s, 3H); ESA-MS: m/z=480.1 [M+H]⁺.

### 5-(3-Cyanophenyl)-2-methyl-N-(3-(3,3,3-trifluoro-2-hydroxy-2-methylpropyl)-1,2,4-thiadiazol-5-yl)furan-3-carboxamide (Compound A-76)

Preparation of compound A-76 referred to the synthesis of compound A-75, using compound A-3 as the starting material to obtain a light yellow solid, yield was 73%; ESA-MS: m/z=437.1 [M+H]⁺.

### 5-(3-Methoxyphenyl)-2-methyl-N-(3-(3,3,3-trifluoro-2-hydroxy-2-methylpropyl)-1,2,4-thiadiazol-5-yl)furan-3-carboxamide (Compound A-77)

Preparation of compound A-77 referred to the synthesis of compound A-75, using compound A-4 as the starting material to obtain a light yellow solid, yield was 73%; ESA-MS: m/z=442.1 [M+H]⁺.

### 2-Methyl-N-(3-(3,3,3-trifluoro-2-hydroxy-2-methylpropyl)-1,2,4-thiadiazol-5-yl)-5-(3-(trifluoromethoxy)phenyl)furan-3-carboxamide (Compound A-78)

Preparation of compound A-78 referred to the synthesis of compound A-75, using compound A-13 as the starting material to obtain a light yellow solid, yield 73%; ESA-MS: m/z=496.1 [M+H]⁺.

### 5-(3-(Difluoromethoxy)phenyl)-2-methyl-N-(3-(3,3,3-trifluoro-2-hydroxy-2-methylpropyl)-1,2,4-thiadiazol-5-yl)furan-3-carboxamide (Compound A-79)

Preparation of compound A-79 referred to the synthesis of compound A-75, using compound A-14 as the starting material to obtain a light yellow solid, yield 73%; ESA-MS: m/z=478.1 [M+H]⁺.

### Example 11: Synthesis of Compounds A-80˜A-84

### N-(3-(3,3-difluoro-2-methylallyl)-1,2,4-thiadiazol-5-yl)-2-methyl-5-(3-(trifluoromethyl)phenyl)furan-3-carboxamide (Compound A-80)

The synthesis route is shown in FIG. 13.

To a solution of compound A-1 (100 mg, 0.24 mmol) and difluoromethyl 2-pyridyl sulfone (57 mg, 0.29 mmol) in anhydrous DMF (4 mL) was added a solution of potassium tert-butoxide (49 mg, 0.44 mmol) in anhydrous DMF (4 mL) at −50° C. under nitrogen atmosphere, and then the mixture was stirred at −40° C. for 1 hour. Saturated ammonium chloride solution (0.4 mL) and 1 mol/L HCl (0.8 mL) was then added to quench the reaction. After moving to room temperature, ethyl acetate was added and the organic layer was washed with water and brine, dried over anhydrous Na₂SO₄, and concentrated to give a residue, which was purified by column-chromatograph to give a light yellow solid, yield 69%; ¹H NMR (500 MHZ, Chloroform-d): δ 8.07 (t. J=1.5 Hz, 1H), 7.78-7.74 (m, 1H), 7.71-7.66 (m, 1H), 7.60 (t, J=7.5 Hz, 1H), 6.99 (s, 1H), 3.29 (s. 2H), 2.53 (s. 3H), 2.02 (s, 3H); ESA-MS: m/z=444.1 [M+H]⁺.

### 5-(3-Cyanophenyl)-N-(3-(3,3-difluoro-2-methylallyl)-1,2,4-thiadiazol-5-yl)-2-methyl-furan-3-carboxamide (Compound A-81)

Preparation of compound A-81 referred to the synthesis of compound A-80, using compound A-3 as the starting material to obtain a light yellow solid, yield was 73%; ESA-MS: m/z=401.1 [M+H]⁺.

### N-(3-(3,3-difluoro-2-methylallyl)-1,2,4-thiadiazol-5-yl)-5-(3-methoxyphenyl)-2-methyl-furan-3-carboxamide (Compound A-82)

Preparation of compound A-82 referred to the synthesis of compound A-80, using compound A-4 as the starting material to obtain a light yellow solid, yield was 66%; ESA-MS: m/z=406.1 [M+H]⁺.

### N-(3-(3,3-difluoro-2-methylallyl)-1,2,4-thiadiazol-5-yl)-2-methyl-5-(3-(trifluoromethoxy)phenyl)furan-3-carboxamide (Compound A-83)

Preparation of compound A-83 referred to the synthesis of compound A-80, using compound A-13 as the starting material to obtain a light yellow solid, yield was 63%; ESA-MS: m/z=460.1 [M+H]⁺.

### N-(3-(3,3-difluoro-2-methylallyl)-1,2,4-thiadiazol-5-yl)-5-(3-(difluoromethoxy)phenyl)-2-methylfuran-3-carboxamide (Compound A-84)

Preparation of compound A-84 referred to the synthesis of compound A-80, using compound A-14 as the starting material to obtain a light yellow solid, yield was 65%; ESA-MS: m/z=442.1 [M+H]⁺.

### Example 12: Synthesis of Compounds A-85˜A-89

### N-(3-(2,2-difluoropropyl)-1,2,4-thiadiazol-5-yl)-2-methyl-5-(3-(trifluoromethyl)phenyl) furan-3-carboxamide (Compound A-85)

The synthesis route is shown in FIG. 14.

To a solution of compound A-1 (100 mg, 0.24 mmol) in anhydrous DCM (5 mL) was added DAST (315 mg. 2.0 mmol) under ice bath. After stirring for a few minutes, the reaction mixture was heated to reflux for 24 hours and then quenched with saturated sodium bicarbonate solution. Ethyl acetate was added and the organic layer was washed with water and brine, dried over anhydrous Na₂SO₄, and concentrated to give a residue, which was purified by column-chromatograph to give a light yellow solid, yield was 55%; ¹H NMR (500 MHZ, Chloroform-d): δ 8.07 (t, J=1.5 Hz, 1H), 7.78-7.74 (m, 1H), 7.70-7.67 (m, 1H), 7.60 (t, J=7.5 Hz, 1H), 6.99 (s, 1H), 3.10 (t, J=21.0 Hz, 2H), 2.59 (s, 3H), 2.22 (t, J=21.0 Hz. 3H); ESA-MS: m/z=432.1 [M+H]⁺.

### 5-(3-Cyanophenyl)-N-(3-(2,2-difluoropropyl)-1,2,4-thiadiazol-5-yl)-2-methylfuran-3-carboxamide (Compound A-86)

Preparation of compound A-86 referred to the synthesis of compound A-85, using compound A-3 as the starting material to obtain a light yellow solid, yield was 65%; ESA-MS: m/z=389.1 [M+H]⁺.

### N-(3-(2,2-difluoropropyl)-1,2,4-thiadiazol-5-yl)-5-(3-methoxyphenyl)-2-methylfuran-3-carboxamide (Compound A-87)

Preparation of compound A-87 referred to the synthesis of compound A-85, using compound A-4 as the starting material to obtain a light yellow solid, yield was 65%; ESA-MS: m/z=394.1 [M+H]⁺.

### N-(3-(2,2-difluoropropyl)-1,2,4-thiadiazol-5-yl)-2-methyl-5-(3-(trifluoromethoxy)phenyl) furan-3-carboxamide (Compound A-88)

Preparation of compound A-88 referred to the synthesis of compound A-85, using compound A-13 as the starting material to obtain a light yellow solid, yield was 65%; ESA-MS: m/z=448.1 [M+H]⁺.

### 5-(3-(Difluoromethoxy)phenyl)-N-(3-(2,2-difluoropropyl)-1,2,4-thiadiazol-5-yl)-2-methyl-furan-3-carboxamide (Compound A-89)

Preparation of compound A-89 referred to the synthesis of compound A-85, using compound A-14 as the starting material to obtain a light yellow solid, yield was 65%; ESA-MS: m/z=430.1 [M+H]⁺.

### Example 13: Synthesis of Compounds A-90˜A-92

### N-(3-(2-hydroxy-2-methylpropyl)-1,2,4-thiadiazol-5-yl)-2-methyl-5-(3-(trifluoromethyl)phenyl)furan-3-carboxamide (Compound A-90)

The synthesis route is shown in FIG. 15.

To a solution of compound A-1 (100 mg, 0.24 mmol) in diethyl ether (5 mL) was added methylmagnesium bromide in diethyl ether (98 μL, 0.29 mmol) dropwise. The reaction mixture was then heated to reflux for 3 hours. After cooling to 0° C. with ice bath, diethyl ether and a small amount of ice was added to dilute the mixture, and then 2 mol/L HCl was added dropwise until all the precipitates were dissolved. The organic layer was washed with water and brine, dried over anhydrous Na₂SO₄, and concentrated to give a residue, which was purified by column-chromatograph to give a light yellow solid, yield was 52%; ¹H NMR (500 MHZ. Chloroform-d): δ 8.09 (t, J=1.5 Hz, 1H), 7.80-7.76 (m, 1H), 7.69-7.65 (m, 1H), 7.61 (t, J=7.5 Hz, 1H), 6.99 (s, 1H), 4.26 (s, 1H), 2.72 (s. 2H), 2.59 (s, 3H), 1.53 (s. 6H); ESA-MS: m/z=426.1 [M+H]⁺.

### N-(3-(2-hydroxy-2-methylpropyl)-1,2,4-thiadiazol-5-yl)-2-methyl-5-(3-(trifluoromethoxy)phenyl)furan-3-carboxamide (Compound A-91)

Preparation of compound A-91 referred to the synthesis of compound A-90, using compound A-13 as the starting material to obtain a light yellow solid, yield was 55%; ESA-MS: m/z=442.1 [M+H]⁺.

### 5-(3-(Difluoromethoxy)phenyl)-N-(3-(2-hydroxy-2-methylpropyl)-1,2,4-thiadiazol-5-yl)-2-methylfuran-3-carboxamide (Compound A-92)

Preparation of compound A-92 referred to the synthesis of compound A-90, using compound A-14 as the starting material to obtain a light yellow solid, yield was 50%; ESA-MS: m/z=424.1 [M+H]⁺.

### Example 14: Synthesis of Compounds A-93˜A-95

### N-(3-(2-amino-2-methylpropyl)-1,2,4-thiadiazol-5-yl)-2-methyl-5-(3-(trifluoromethyl)phenyl)furan-3-carboxamide (Compound A-93)

The synthesis route is shown in FIG. 16.

### (a) Synthesis of N-(3-(2-(2-chloroacetamido)-2-methylpropyl)-1,2,4-thiadiazol-5-yl)-2-methyl-5-(3-(trifluoromethyl)phenyl)furan-3-carboxamide (Compound V-2-1)

To a solution of compound A-90/V-1-1 (100 mg, 0.24 mmol) and 2-chloroacetonitrile (94 mg, 1.5 mmol) in acetic acid (2 mL) was added concentrated sulfuric acid (116 μL, 2.2 mmol) dropwise at 0° C. The reaction mixture was then moved to room temperature and stirred for 5 hours. Ethyl acetate was added and the organic layer was washed with water and brine, dried over anhydrous Na₂SO₄, and concentrated to give a residue, which was purified by column-chromatograph to give a yellow solid, yield was 82%; ESA-MS: m/z=501.1 [M+H]⁺.

### (b) Synthesis of N-(3-(2-amino-2-methylpropyl)-1,2,4-thiadiazol-5-yl)-2-methyl-5-(3-(tri-fluoromethyl)phenyl)furan-3-carboxamide (Compound A-93)

To a solution of compound V-2-1 (75 mg, 0.15 mmol) in acetic acid and ethanol (6 mL, 1:5) was added thiourea (15 mg, 0.20 mmol). The reaction was then heated to reflux overnight and alkalized with saturated sodium bicarbonate solution. Ethyl acetate was added and the organic layer was washed with water and brine, dried over anhydrous Na₂SO₄, and concentrated to give a residue, which was purified by column-chromatograph to give a yellow solid, yield was 78%; ¹H NMR (500 MHZ, Chloroform-d): δ 8.11 (t, J=1.5 Hz, 1H), 7.81-7.77 (m, 1H), 7.70-7.66 (m, 1H), 7.63 (t, J=7.5 Hz, 1H), 7.02 (s, 1H), 2.65 (s. 2H), 2.61 (s, 3H), 2.39 (s, 2H), 1.35 (s, 6H); ESA-MS: m/z=425.1 [M+H]⁺.

### N-(3-(2-amino-2-methylpropyl)-1,2,4-thiadiazol-5-yl)-2-methyl-5-(3-(trifluoromethoxy)phenyl)furan-3-carboxamide (Compound A-94)

Preparation of compound A-94 referred to the synthesis of compound A-93, using compound A-91 as the starting material to obtain a light yellow solid, yield was 52%; ESA-MS: m/z=441.1 [M+H]⁺.

### N-(3-(2-amino-2-methylpropyl)-1,2,4-thiadiazol-5-yl)-5-(3-(difluoromethoxy)phenyl)-2-methylfuran-3-carboxamide (Compound A-95)

Preparation of compound A-95 referred to the synthesis of compound A-93, using compound A-92 as the starting material to obtain a light yellow solid, yield was 58%; ESA-MS: m/z=423.1 [M+H]⁺.

### Example 15: Synthesis of Compounds A-96˜A-98

### 2-Methyl-N-(3-(2-(methylamino) propyl)-1,2,4-thiadiazol-5-yl)-5-(3-(trifluoromethyl)phenyl)furan-3-carboxamide (Compound A-96)

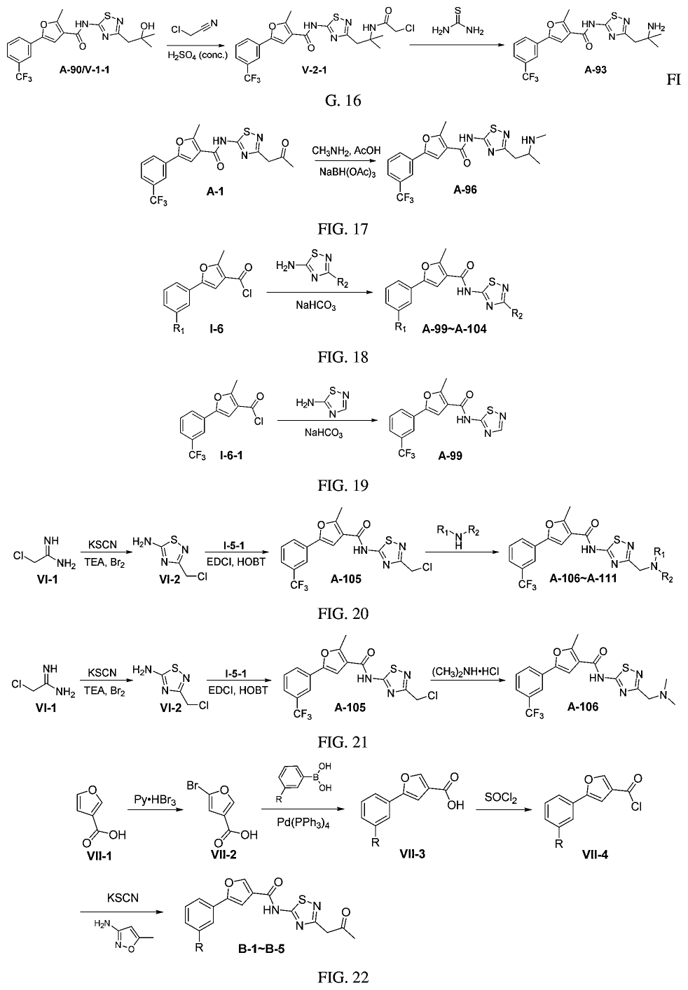

The synthesis route is shown in FIG. 17.

To a solution of compound A-1 (100 mg, 0.24 mmol) in DCM (5 mL) was added 40% methylamine aqueous solution (43 μL, 0.49 mmol) and acetic acid (22 mg, 0.37 mmol). After stirring at room temperature overnight, sodium triacetoxyborohydride (52 mg, 0.24 mmol) was added and stirred for another 4 hours. The reaction mixture was then alkalized with saturated sodium bicarbonate solution. Ethyl acetate was added and the organic layer was washed with water and brine, dried over anhydrous Na₂SO₄, and concentrated to give a residue, which was purified by column-chromatograph to give a light yellow solid, yield was 62%; ¹H NMR (500 MHZ, Chloroform-d): δ 8.09 (t, J=1.5 Hz, 1H), 7.78-7.74 (m, 1H), 7.69-7.65 (m, 1H), 7.62 (t, J=7.5 Hz, 1H), 7.01 (s, 1H), 3.86-3.82 (m, 1H), 3.67-3.63 (m, 1H), 2.74 (dd, J₁=12.5 Hz, J₂=7.0 Hz, 1H), 2.67-2.58 (m, 4H), 2.42 (d, J=3.0 Hz, 3H), 1.23 (d, J=7.0 Hz, 3H); ESA-MS: m/z=425.1 [M+H]⁺.

### 5-(3-Cyanophenyl)-2-methyl-N-(3-(2-(methylamino) propyl)-1,2,4-thiadiazol-5-yl)furan-3-carboxamide (Compound A-97)

Preparation of compound A-97 referred to the synthesis of compound A-96, using compound A-3 as the starting material to obtain a light yellow solid, yield was 55%; ESA-MS: m/z=382.1 [M+H]⁺.

### 2-Methyl-N-(3-(2-(methylamino) propyl)-1,2,4-thiadiazol-5-yl)-5-(3-(trifluoromethoxy)phenyl)furan-3-carboxamide (Compound A-98)

Preparation of compound A-98 referred to the synthesis of compound A-96, using compound A-13 as the starting material to obtain a light yellow solid, yield was 53%; ESA-MS: m/z=441.1 [M+H]⁺.

### Example 16: Synthesis of Compounds A-99˜A-104

The synthesis route is shown in FIG. 18.

### 2-Methyl-N-(1,2,4-thiadiazol-5-yl)-5-(3-(trifluoromethyl)phenyl)furan-3-carboxamide (Compound A-99)

The specific synthesis route is shown in FIG. 19.

A solution of compound I-6-1 (200 mg, 0.69 mmol), 1,2,4-thiadiazol-5-amine (70 mg, 0.69 mmol) and sodium bicarbonate (175 mg, 2.1 mmol) in anhydrous acetonitrile (10 mL) was heated at 60° C. for 1 hour. Ethyl acetate was added and the organic layer was washed with water and brine, dried over anhydrous Na₂SO₄, and concentrated to give a residue, which was purified by column-chromatograph to give a light yellow solid, yield was 81%; ¹H NMR (500 MHZ, Chloroform-d): δ 8.36 (s, 1H), 8.09 (t, J=1.5 Hz, 1H), 7.75-7.71 (m, 2H), 7.61 (t, J=7.5 Hz, 1H), 7.02 (s, 1H), 2.55 (s, 3H); ESA-MS: m/z=354.0 [M+H]⁺.

### 2-Methyl-N-(3-methyl-1,2,4-thiadiazol-5-yl)-5-(3-(trifluoromethyl)phenyl)furan-3-carboxamide (Compound A-100)

Preparation of compound A-100 referred to the synthesis of compound A-99, replacing 1,2,4-thiadiazol-5-amine with 3-methyl-1,2,4-thiadiazol-5-amine to obtain a light yellow solid, yield was 79%; 1H NMR (500 MHZ, Chloroform-d): δ 8.10 (t, J=1.5 Hz, 1H), 7.78-7.74 (m, 2H), 7.62 (t, J=7.5 Hz, 1H), 7.02 (s, 1H), 2.61 (s, 3H), 2.42 (s, 3H); ESA-MS: m/z=368.1 [M+H]⁺.

### 2-Methyl-N-(1,2,4-thiadiazol-5-yl)-5-(3-(trifluoromethoxy)phenyl)furan-3-carboxamide (Compound A-101)

Preparation of compound A-101 referred to the synthesis of compound A-99, using 2-methyl-5-(3-(trifluoromethoxy)phenyl)furan-3-carbonyl chloride (compound I-6-13) as the starting material to obtain a light yellow solid, yield was 77%; ESA-MS: m/z=370.0 [M+H]⁺.

### 2-Methyl-N-(3-methyl-1,2,4-thiadiazol-5-yl)-5-(3-(trifluoromethoxy)phenyl)furan-3-carboxamide (Compound A-102)

Preparation of compound A-102 referred to the synthesis of compound A-101, replacing 1,2,4-thiadiazol-5-amine with 3-methyl-1,2,4-thiadiazol-5-amine to obtain a light yellow solid, yield was 71%; ESA-MS: m/z=384.1 [M+H]⁺.

### 5-(3-(Difluoromethoxy)phenyl)-2-methyl-N-(1,2,4-thiadiazol-5-yl)furan-3-carboxamide 2-(Compound A-103)

Preparation of compound A-103 referred to the synthesis of compound A-99, using 2-methyl-5-(3-(difluoromethoxy)phenyl)furan-3-carbonyl chloride (compound I-6-14) as the starting material to obtain a light yellow solid, yield was 80%; ESA-MS: m/z=352.0 [M+H]⁺.

### 5-(3-(Difluoromethoxy)phenyl)-2-methyl-N-(3-methyl-1,2,4-thiadiazol-5-yl)furan-3-carboxamide (Compound A-104)

Preparation of compound A-104 referred to the synthesis of compound A-103, replacing 1,2,4-thiadiazol-5-amine with 3-methyl-1,2,4-thiadiazol-5-amine to obtain a light yellow solid, yield was 75%; ESA-MS: m/z=366.1 [M+H]⁺.

### Example 17: Synthesis of Compounds A-105˜A-111

The synthesis route is shown in FIG. 20.

### N-(3-(chloromethyl)-1,2,4-thiadiazol-5-yl)-2-methyl-5-(3-(trifluoromethyl)phenyl)furan-3-carboxamide (Compound A-105)

The specific synthesis route is shown in FIG. 21.

### (a) Synthesis of 3-(chloromethyl)-1,2,4-thiadiazol-5-amine (Compound VI-2)

A solution of 2-chloroacetamidine hydrochloride (VI-1, 1.00 g, 7.8 mmol) in anhydrous methanol (15 mL) was cooled to 0° C., and triethylamine (1.96 g, 19.4 mmol) and bromine (1.12 g, 7.0 mmol) was added. A solution of potassium thiocyanate (829 mg, 8.5 mmol) in methanol (8 mL) was then added dropwise, and the mixture was stirred at 0° C. for 2 hours. Ethyl acetate was added and the organic layer was washed with water and brine, dried over anhydrous Na₂SO₄, and concentrated to give a residue, which was purified by column-chromatograph to give a white solid, yield was 62%; ESA-MS: m/z=150.0 [M+H]⁺.

### (b) Synthesis of N-(3-(chloromethyl)-1,2,4-thiadiazol-5-yl)-2-methyl-5-(3-(trifluoromethyl)phenyl)furan-3-carboxamide (Compound A-105)

To a solution of compound I-5-1 (1.3 g, 4.8 mmol) in DMA (10 mL) was added EDCI (923 mg, 4.8 mmol), triethylamine (1.22 g, 12.0 mmol) and HOBT (596 mg, 4.4 mmol) at 0° C. and stirred for 30 min. Compound VI-1 (600 mg, 4.0 mmol) was then added, and the reaction mixture was heated to 50° C. and stirred for 3 hours. Ethyl acetate was added and the organic layer was washed with water and brine, dried over anhydrous Na₂SO₄, and concentrated to give a residue, which was purified by column-chromatograph to give a light yellow solid, yield was 52%; ¹H NMR (500 MHZ, Chloroform-d): δ 8.07 (t, J=1.5 Hz, 1H), 7.81-7.76 (m, 1H), 7.68-7.64 (m. 1H), 7.61 (t, J=7.5 Hz, 1H), 7.00 (s, 1H), 4.61 (s. 2H), 2.59 (s, 3H); ESA-MS: m/z=402.0 [M+H]⁺.

### N-(3-((dimethylamino)methyl)-1,2,4-thiadiazol-5-yl)-2-methyl-5-(3-(trifluoromethyl)phenyl)furan-3-carboxamide (Compound A-106)

To a solution of compound A-105 (100 mg, 0.25 mmol) in acetonitrile (10 mL) was added dimethylamine hydrochloride (41 mg, 0.50 mmol) and triethylamine (76 mg, 0.75 mmol) and heated to reflux for 4 hours. Ethyl acetate was added and the organic layer was washed with water and brine, dried over anhydrous Na₂SO₄, and concentrated to give a residue, which was purified by column-chromatograph to give a light yellow solid, yield was 82%; ¹H NMR (500 MHZ, Chloroform-d): δ 8.07 (t, J=1.5 Hz, 1H), 7.80-7.76 (m, 1H), 7.68-7.64 (m, 1H). 7.60 (t, J=7.5 Hz, 1H), 6.99 (s, 1H), 4.40 (s. 2H), 2.59 (s, 3H), 2.28 (s. 6H); ESA-MS: m/z=411.1 [M+H]⁺.

### N-(3-((diethylamino)methyl)-1,2,4-thiadiazol-5-yl)-2-methyl-5-(3-(trifluoromethyl)phenyl)furan-3-carboxamide (Compound A-107)

Preparation of compound A-107 referred to the synthesis of compound A-106, replacing dimethylamine hydrochloride with diethylamine hydrochloride to obtain a white solid, yield was 80%; ESA-MS: m/z=439.1 [M+H]⁺.

### 2-Methyl-N-(3-(pyrrolidin-1-ylmethyl)-1,2,4-thiadiazol-5-yl)-5-(3-(trifluoromethyl)phenyl)furan-3-carboxamide (Compound A-108)

Preparation of compound A-108 referred to the synthesis of compound A-106, replacing dimethylamine hydrochloride with pyrrolidine to obtain a white solid, yield was 75%; ESA-MS: m/z=437.1 [M+H]⁺. 2-Methyl-N-(3-(piperidin-1-ylmethyl)-1,2,4-thiadiazol-5-yl)-5-(3-(trifluoromethyl)phenyl)furan-3-carbo xamide (Compound A-109)

Preparation of compound A-109 referred to the synthesis of compound A-106, replacing dimethylamine hydrochloride with piperidine to obtain a light yellow solid, yield was 70%; ESA-MS: m/z=451.1 [M+H]⁺.

### 2-Methyl-N-(3-(morpholinomethyl)-1,2,4-thiadiazol-5-yl)-5-(3-(trifluoromethyl)phenyl) furan-3-carboxamide (Compound A-110)

Preparation of compound A-110 referred to the synthesis of compound A-106, replacing dimethylamine hydrochloride with morpholine to obtain a white solid, yield was 80%; ESA-MS: m/z=453.1 [M+H]⁺.

### 2-Methyl-N-(3-((4-methylpiperazin-1-yl)methyl)-1,2,4-thiadiazol-5-yl)-5-(3-(trifluoro-methyl)phenyl)furan-3-carboxamide (Compound A-111)

Preparation of compound A-111 referred to the synthesis of compound A-106, replacing dimethylamine hydrochloride with 1-methylpiperazine to obtain a light yellow solid, yield was 78%; ESA-MS: m/z=466.1 [M+H]⁺.

### Example 18: Synthesis of Compounds A-112˜A-131

### N-(3-(chloromethyl)-1,2,4-thiadiazol-5-yl)-5-(3-cyanophenyl)-2-methylfuran-3-carboxamide (Compound A-112)

Preparation of compound A-112 referred to the synthesis of compound A-105, replacing 2-methyl-5-(3-(trifluoromethyl)phenyl)furan-3-carboxylic acid (compound I-5-1) with 5-(3-cyanophenyl)-2-methylfuran-3-carboxylic acid (compound I-5-3) to obtain a light yellow solid, yield was 29%; ESA-MS: m/z=359.0 [M+H]⁺.

### 5-(3-Cyanophenyl)-N-(3-((dimethylamino)methyl)-1,2,4-thiadiazol-5-yl)-2-methylfuran-3-carboxamid (Compound A-113)

Preparation of compound A-113 referred to the synthesis of compound A-106, replacing compound A-105 with compound A-112 to obtain a light yellow solid, yield was 78%; ESA-MS: m/z=368.1 [M+H]⁺.

### 5-(3-Cyanophenyl)-2-methyl-N-(3-(piperidin-1-ylmethyl)-1,2,4-thiadiazol-5-yl)furan-3-carboxamide (Compound A-114)

Preparation of compound A-114 referred to the synthesis of compound A-109, replacing compound A-105 with compound A-112 to obtain a light yellow solid, yield was 77%; ESA-MS: m/z=408.1 [M+H]⁺.

### 5-(3-Cyanophenyl)-2-methyl-N-(3-(morpholinomethyl)-1,2,4-thiadiazol-5-yl)furan-3-carboxamide (Compound A-115)

Preparation of compound A-115 referred to the synthesis of compound A-110, replacing compound A-105 with compound A-112 to obtain a white solid, yield was 80%; ESA-MS: m/z=410.1 [M+H]⁺.

### 5-(3-Cyanophenyl)-2-methyl-N-(3-((4-methylpiperazin-1-yl)methyl)-1,2,4-thiadiazol-5-yl)furan-3-car boxamide (Compound A-116)

Preparation of compound A-116 referred to the synthesis of compound A-111, replacing compound A-105 with compound A-112 to obtain a light yellow solid, yield was 78%; ESA-MS: m/z=423.2 [M+H]⁺.

### N-(3-(chloromethyl)-1,2,4-thiadiazol-5-yl)-5-(3-methoxyphenyl)-2-methylfuran-3-carboxamide (Compound A-117)

Preparation of compound A-117 referred to the synthesis of compound A-105, replacing 2-methyl-5-(3-(trifluoromethyl)phenyl)furan-3-carboxylic acid (compound I-5-1) with 5-(3-methoxyphenyl)-2-methylfuran-3-carboxylic acid (compound I-5-4) to obtain a light yellow solid, yield was 35%; ESA-MS: m/z=364.0 [M+H]⁺.

### N-(3-((dimethylamino)methyl)-1,2,4-thiadiazol-5-yl)-5-(3-methoxyphenyl)-2-methylfuran-3-carboxamide (Compound A-118)

Preparation of compound A-118 referred to the synthesis of compound A-106, replacing compound A-105 with compound A-117 to obtain a light yellow solid, yield was 79%; ESA-MS: m/z=373.1 [M+H]⁺.

### N-(3-((diethylamino)methyl)-1,2,4-thiadiazol-5-yl)-5-(3-methoxyphenyl)-2-methylfuran-3-carboxamide (Compound A-119)

Preparation of compound A-119 referred to the synthesis of compound A-107, replacing compound A-105 with compound A-117 to obtain a light yellow solid, yield was 86%; ESA-MS: m/z=401.2 [M+H]⁺.

### 5-(3-Methoxyphenyl)-2-methyl-N-(3-(morpholinomethyl)-1,2,4-thiadiazol-5-yl)furan-3-carboxamide (Compound A-120)

Preparation of compound A-120 referred to the synthesis of compound A-110, replacing compound A-105 with compound A-117 to obtain a white solid, yield was 80%; ESA-MS: m/z=415.1 [M+H]⁺.

### 5-(3-Methoxyphenyl)-2-methyl-N-(3-((4-methylpiperazin-1-yl)methyl)-1,2,4-thiadiazol-5-yl)furan-3-carboxamide (Compound A-121)

Preparation of compound A-121 referred to the synthesis of compound A-111, replacing compound A-105 with compound A-117 to obtain a light yellow solid, yield was 78%; ESA-MS: m/z=428.2 [M+H]⁺.

### N-(3-(chloromethyl)-1,2,4-thiadiazol-5-yl)-2-methyl-5-(3-(trifluoromethoxy)phenyl)furan-3-carboxamide (Compound A-122)

Preparation of compound A-122 referred to the synthesis of compound A-105, replacing 2-methyl-5-(3-(trifluoromethyl)phenyl)furan-3-carboxylic acid (compound I-5-1) with 2-methyl-5-(3-(trifluoromethoxy)phenyl)furan-3-carboxylic acid (compound I-5-13) to obtain a light yellow solid, yield was 21%; ESA-MS: m/z=418.0 [M+H]⁺.

### N-(3-((diethylamino)methyl)-1,2,4-thiadiazol-5-yl)-2-methyl-5-(3-(trifluoromethoxy)phenyl)furan-3-carboxamide (Compound A-123)

Preparation of compound A-123 referred to the synthesis of compound A-107, replacing compound A-105 with compound A-122 to obtain a light yellow solid, yield was 75%; ESA-MS: m/z=455.1 [M+H]⁺.

### 2-Methyl-N-(3-(pyrrolidin-1-ylmethyl)-1,2,4-thiadiazol-5-yl)-5-(3-(trifluoromethoxy)phenyl)furan-3-carboxamide (Compound A-124)

Preparation of compound A-124 referred to the synthesis of compound A-108, replacing compound A-105 with compound A-122 to obtain a light yellow solid, yield was 69%; ESA-MS: m/z=453.1 [M+H]⁺.

### 2-Methyl-N-(3-(morpholinomethyl)-1,2,4-thiadiazol-5-yl)-5-(3-(trifluoromethoxy)phenyl) furan-3-carboxamide (Compound A-125)

Preparation of compound A-125 referred to the synthesis of compound A-110, replacing compound A-105 with compound A-122 to obtain a white solid, yield was 70%; ESA-MS: m/z=469.1 [M+H]⁺.

### 2-Methyl-N-(3-((4-methylpiperazin-1-yl)methyl)-1,2,4-thiadiazol-5-yl)-5-(3-(trifluoro-methoxy)phenyl)furan-3-carboxamide (Compound A-126)

Preparation of compound A-126 referred to the synthesis of compound A-111, replacing compound A-105 with compound A-122 to obtain a light yellow solid, yield was 74%; ESA-MS: m/z=482.1 [M+H]⁺.

### N-(3-(chloromethyl)-1,2,4-thiadiazol-5-yl)-5-(3-(difluoromethoxy)phenyl)-2-methylfuran-3-carboxamide (Compound A-127)

Preparation of compound A-127 referred to the synthesis of compound A-105, replacing 2-methyl-5-(3-(trifluoromethyl)phenyl)furan-3-carboxylic acid (compound I-5-1) with 5-(3-(difluoromethoxy)phenyl)-2-methylfuran-3-carboxylic acid (compound I-5-14) to obtain a light yellow solid, yield was 24%; ESA-MS: m/z=400.0 [M+H]⁺.

### 5-(3-(Difluoromethoxy)phenyl)-2-methyl-N-(3-(pyrrolidin-1-ylmethyl)-1,2,4-thiadiazol-5-yl)furan-3-carboxamide (Compound A-128)

Preparation of compound A-128 referred to the synthesis of compound A-108, replacing compound A-105 with compound A-127 to obtain a light yellow solid, yield was 82%; ESA-MS: m/z=435.1 [M+H]⁺.

### 5-(3-(Difluoromethoxy)phenyl)-2-methyl-N-(3-(piperidin-1-ylmethyl)-1,2,4-thiadiazol-5-yl)furan-3-carboxamide (Compound A-129)

Preparation of compound A-129 referred to the synthesis of compound A-109, replacing compound A-105 with compound A-127 to obtain a light yellow solid, yield was 76%; ESA-MS: m/z=449.1 [M+H]⁺.

### 5-(3-(Difluoromethoxy)phenyl)-2-methyl-N-(3-(morpholinomethyl)-1,2,4-thiadiazol-5-yl)furan-3-carboxamide (Compound A-130)

Preparation of compound A-130 referred to the synthesis of compound A-110, replacing compound A-105 with compound A-127 to obtain a white solid, yield was 80%; ESA-MS: m/z=451.1 [M+H]⁺.

### 5-(3-(Difluoromethoxy)phenyl)-2-methyl-N-(3-((4-methylpiperazin-1-yl)methyl)-1,2,4-thiadiazol-5-yl)furan-3-carboxamide (Compound A-131)

Preparation of compound A-131 referred to the synthesis of compound A-111, replacing compound A-105 with compound A-127 to obtain a light yellow solid, yield was 77%; ESA-MS: m/z=464.1 [M+H]⁺.

### Example 19: Synthesis of Compounds B-1˜B-5

The synthesis route is shown in FIG. 22.

### N-(3-(2-oxopropyl)-1,2,4-thiadiazol-5-yl)-5-(3-(trifluoromethyl)phenyl)furan-3-carboxamide (Compound B-1)

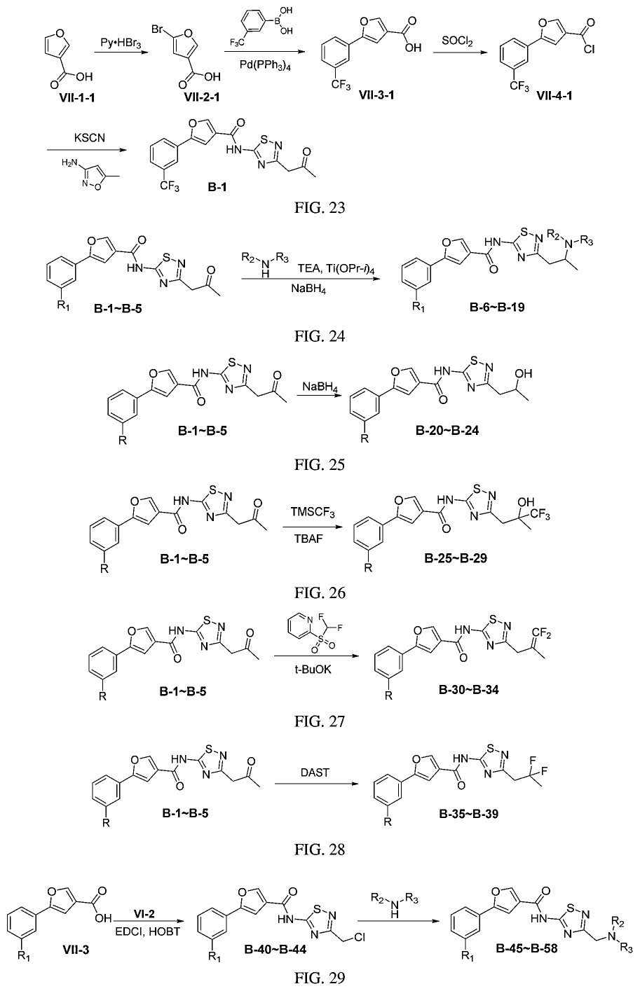

The specific synthesis route is shown in FIG. 23.

### (a) Synthesis of 5-bromofuran-3-carboxylic acid (Compound VII-2-1)

To a solution of pyridinium tribromide (2.88 g, 9.0 mmol) in acetic acid (4 mL) was added 3-furoic acid (VII-1-1, 1.04 g, 9.3 mmol). The reaction mixture was stirred at 40° C. for 24 hours and neutralized with saturated sodium carbonate solution. Ethyl acetate was added and the organic layer was washed with water and brine, dried over anhydrous Na₂SO₄, and concentrated to give a residue, which was purified by column-chromatograph to give a yellow solid, yield was 58%; ESA-MS: m/z=191.0 [M+H]⁺.

### (b) Synthesis of 5-(3-(trifluoromethyl)phenyl)furan-3-carboxylic acid (Compound VII-3-1)

A suspension of compound VII-2-1 (500 mg, 2.6 mmol), 3-(trifluoromethyl)phenylboronic acid (497 mg, 2.6 mmol) and tetrakis(triphenylphosphine)palladium (151 mg, 0.13 mmol) in DCE (6 mL) and 2 mol/L sodium carbonate solution (3 mL) was stirred at 95° C. for 4 hours. After cooling to the room temperature, ethyl acetate was added and the organic layer was washed with water and brine, dried over anhydrous Na₂SO₄, and concentrated to give a residue, which was purified by column-chromatograph to give a light yellow solid, yield was 58%; ESA-MS: m/z=257.0 [M+H]⁺.

### (c) Synthesis of 5-(3-(trifluoromethyl)phenyl)furan-3-carbonyl chloride (Compound VII-4-1)

A solution of compound VII-3-1 (387 mg, 1.5 mmol) in thionyl chloride (6 mL) was heated to reflux for 2.5 hours, and the mixture was concentrated to dryness to get a light brown solid, yield was 95%;

### (d) Synthesis of N-(3-(2-oxopropyl)-1,2,4-thiadiazol-5-yl)-5-(3-(trifluoromethyl)phenyl)furan-3-carboxamide (Compound B-1)

A suspension of compound VII-4-1 (394 mg, 1.4 mmol) and potassium thiocyanate (153 mg, 1.6 mmol) in acetonitrile (10 mL) was stirred at 60° C. for 30 min, and then 5-methylisoxazol-3-amine (141 mg, 1.4 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 80%; ¹H NMR (500 MHZ, Chloroform-d): δ 8.18 (d, J=1.5 Hz, 1H), 8.10 (t, J=1.5 Hz, 1H), 7.89-7.85 (m, 1H), 7.76-7.72 m, 1H), 7.63 (t, J=7.5 Hz, 1H), 7.36 (d, J=1.5 Hz, 1H), 3.57 (s, 2H), 2.30 (s, 3H); ESA-MS: m/z=396.1 [M+H]⁺.

### 5-(3-Cyanophenyl)-N-(3-(2-oxopropyl)-1,2,4-thiadiazol-5-yl)furan-3-carboxamide (Compound B-2)

Preparation of compound B-2 referred to the synthesis of compound B-1, replacing 3-(trifluoromethyl)phenylboronic acid with (3-cyanophenyl) boronic acid to obtain a light yellow solid, yield was 22%; ESA-MS: m/z=353.1 [M+H]⁺.

### 5-(3-Methoxyphenyl)-N-(3-(2-oxopropyl)-1,2,4-thiadiazol-5-yl)furan-3-carboxamide (Compound B-3)

Preparation of compound B-3 referred to the synthesis of compound B-1, replacing 3-(trifluoromethyl)phenylboronic acid with (3-methoxyphenyl) boronic acid to obtain a light yellow solid, yield was 25%; ESA-MS: m/z=358.1 [M+H]⁺.

### N-(3-(2-oxopropyl)-1,2,4-thiadiazol-5-yl)-5-(3-(trifluoromethoxy)phenyl)furan-3-carboxamide (Compound B-4)

Preparation of compound B-4 referred to the synthesis of compound B-1, replacing 3-(trifluoromethyl)phenylboronic acid with (3-(trifluoromethoxy)phenyl) boronic acid to obtain a light yellow solid, yield was 32%; ESA-MS: m/z=412.0 [M+H]⁺.

### 5-(3-(Difluoromethoxy)phenyl)-N-(3-(2-oxopropyl)-1,2,4-thiadiazol-5-yl)furan-3-carboxamide (Compound B-5)

Preparation of compound B-5 referred to the synthesis of compound B-1, replacing 3-(trifluoromethyl)phenylboronic acid with (3-(difluoromethoxy)phenyl) boronic acid to obtain a light yellow solid, yield was 33%; ESA-MS: m/z=394.1 [M+H]⁺.

### Example 20: Synthesis of Compounds B-6˜B-19

The synthesis route is shown in FIG. 24.

### N-(3-(2-(dimethylamino)propyl)-1,2,4-thiadiazol-5-yl)-5-(3-(trifluoromethyl)phenyl) furan-3-carboxamide (Compound B-6)

Preparation of compound B-6 referred to the synthesis of compound A-38, using compound B-1 and dimethylamine hydrochloride as the starting material to obtain a light yellow solid, yield was 73%; ESA-MS: m/z=425.1 [M+H]⁺.

### N-(3-(2-(diethylamino)propyl)-1,2,4-thiadiazol-5-yl)-5-(3-(trifluoromethyl)phenyl)furan-3-carboxamide (Compound B-7)

Preparation of compound B-7 referred to the synthesis of compound A-38, using compound B-1 and diethylamine hydrochloride as the starting material to obtain a light yellow solid, yield was 69%; ESA-MS: m/z=453.1 [M+H]⁺.

### N-(3-(2-(pyrrolidin-1-yl)propyl)-1,2,4-thiadiazol-5-yl)-5-(3-(trifluoromethyl)phenyl)furan-3-carboxamide (Compound B-8)

Preparation of compound B-8 referred to the synthesis of compound A-38, using compound B-1 and pyrrolidine as the starting material to obtain a light yellow solid, yield was 65%; ESA-MS: m/z=451.1 [M+H]⁺.

### N-(3-(2-(piperidin-1-yl)propyl)-1,2,4-thiadiazol-5-yl)-5-(3-(trifluoromethyl)phenyl)furan-3-carboxamide (Compound B-9)

Preparation of compound B-9 referred to the synthesis of compound A-38, using compound B-1 and piperidine as the starting material to obtain a light yellow solid, yield was 72%; ESA-MS: m/z=465.1 [M+H]⁺.

### N-(3-(2-morpholinopropyl)-1,2,4-thiadiazol-5-yl)-5-(3-(trifluoromethyl)phenyl)furan-3-carboxamide (Compound B-10)

Preparation of compound B-10 referred to the synthesis of compound A-38, using compound B-1 and morpholine as the starting material to obtain a light yellow solid, yield was 77%; ESA-MS: m/z=467.1 [M+H]⁺.

### N-(3-(2-(4-methylpiperazin-1-yl)propyl)-1,2,4-thiadiazol-5-yl)-5-(3-(trifluoromethyl)phenyl)furan-3-carboxamide (Compound B-11)

Preparation of compound B-11 referred to the synthesis of compound A-38, using compound B-1 and 1-methylpiperazine as the starting material to obtain a light yellow solid, yield was 69%; ESA-MS: m/z=480.2 [M+H]⁺.

### 5-(3-Cyanophenyl)-N-(3-(2-(pyrrolidin-1-yl)propyl)-1,2,4-thiadiazol-5-yl)furan-3-carboxamide (Compound B-12)

Preparation of compound B-12 referred to the synthesis of compound A-38, using compound B-2 and pyrrolidine as the starting material to obtain a light yellow solid, yield was 66%; ESA-MS: m/z=408.1 [M+H]⁺.

### 5-(3-Cyanophenyl)-N-(3-(2-morpholinopropyl)-1,2,4-thiadiazol-5-yl)furan-3-carboxamide (Compound B-13)

Preparation of compound B-13 referred to the synthesis of compound A-38, using compound B-2 and morpholine as the starting material to obtain a light yellow solid, yield was 72%; ESA-MS: m/z=424.1 [M+H]⁺.

### 5-(3-Methoxyphenyl)-N-(3-(2-(piperidin-1-yl)propyl)-1,2,4-thiadiazol-5-yl)furan-3-carboxamide (Compound B-14)

Preparation of compound B-14 referred to the synthesis of compound A-38, using compound B-3 and piperidine as the starting material to obtain a light yellow solid, yield was 75%; ESA-MS: m/z=427.2 [M+H]⁺.

### 5-(3-Methoxyphenyl)-N-(3-(2-morpholinopropyl)-1,2,4-thiadiazol-5-yl)furan-3-carboxamide (Compound B-15)

Preparation of compound B-15 referred to the synthesis of compound A-38, using compound B-3 and morpholine as the starting material to obtain a light yellow solid, yield was 65%; ESA-MS: m/z=429.2 [M+H]⁺.

### N-(3-(2-morpholinopropyl)-1,2,4-thiadiazol-5-yl)-5-(3-(trifluoromethoxy)phenyl)furan-3-carboxamide (Compound B-16)

Preparation of compound B-16 referred to the synthesis of compound A-38, using compound B-4 and morpholine as the starting material to obtain a light yellow solid, yield was 66%; ESA-MS: m/z=483.1 [M+H]⁺.

### N-(3-(2-(4-methylpiperazin-1-yl)propyl)-1,2,4-thiadiazol-5-yl)-5-(3-(trifluoromethoxy)phenyl)furan-3-carboxamide (Compound B-17)

Preparation of compound B-17 referred to the synthesis of compound A-38, using compound B-4 and 1-methylpiperazine as the starting material to obtain a light yellow solid, yield was 75%; ESA-MS: m/z=496.2 [M+H]⁺.

### 5-(3-(Difluoromethoxy)phenyl)-N-(3-(2-morpholinopropyl)-1,2,4-thiadiazol-5-yl)furan-3-carboxamide (Compound B-18)

Preparation of compound B-18 referred to the synthesis of compound A-38, using compound B-5 and morpholine as the starting material to obtain a light yellow solid, yield was 77%; ESA-MS: m/z=465.1 [M+H]⁺.

### 5-(3-(Difluoromethoxy)phenyl)-N-(3-(2-(4-methylpiperazin-1-yl)propyl)-1,2,4-thiadiazol-5-yl)furan-3-carboxamide (Compound B-19)

Preparation of compound B-19 referred to the synthesis of compound A-38, using compound B-5 and 1-methylpiperazine as the starting material to obtain a light yellow solid, yield was 65%; ESA-MS: m/z=478.2 [M+H]⁺.

### Example 21: Synthesis of Compounds B-20˜B-24

The synthesis route is shown in FIG. 25.

### N-(3-(2-hydroxypropyl)-1,2,4-thiadiazol-5-yl)-5-(3-(trifluoromethyl)phenyl)furan-3-carboxamide (compound B-20)

Preparation of compound B-20 referred to the synthesis of compound A-60, using compound B-1 as the starting material to obtain a light yellow solid, yield was 85%; ESA-MS: m/z=398.1 [M+H]⁺.

### 5-(3-Cyanophenyl)-N-(3-(2-hydroxypropyl)-1,2,4-thiadiazol-5-yl)furan-3-carboxamide (Compound B-21)

Preparation of compound B-21 referred to the synthesis of compound A-60, using compound B-2 as the starting material to obtain a light yellow solid, yield was 81%; ESA-MS: m/z=355.1 [M+H]⁺.

### N-(3-(2-hydroxypropyl)-1,2,4-thiadiazol-5-yl)-5-(3-methoxyphenyl)furan-3-carboxamide (Compound B-22)

Preparation of compound B-22 referred to the synthesis of compound A-60, using compound B-3 as the starting material to obtain a light yellow solid, yield was 78%; ESA-MS: m/z=360.1 [M+H]⁺.

### N-(3-(2-hydroxypropyl)-1,2,4-thiadiazol-5-yl)-5-(3-(trifluoromethoxy)phenyl)furan-3-carboxamide (Compound B-23)

Preparation of compound B-23 referred to the synthesis of compound A-60, using compound B-4 as the starting material to obtain a light yellow solid, yield was 75%; ESA-MS: m/z=414.1 [M+H]⁺.

### 5-(3-(Difluoromethoxy)phenyl)-N-(3-(2-hydroxypropyl)-1,2,4-thiadiazol-5-yl)furan-3-carboxamide (Compound B-24)

Preparation of compound B-24 referred to the synthesis of compound A-60, using compound B-5 as the starting material to obtain a light yellow solid, yield was 80%; ESA-MS: m/z=396.1 [M+H]⁺.

### Example 22: Synthesis of Compounds B-25˜B-29

The synthesis route is shown in FIG. 26.

### N-(3-(3,3,3-trifluoro-2-hydroxy-2-methylpropyl)-1,2,4-thiadiazol-5-yl)-5-(3-(trifluoro-methyl)phenyl)furan-3-carboxamide (Compound B-25)

Preparation of compound B-25 referred to the synthesis of compound A-75, using compound B-1 as the starting material to obtain a light yellow solid, yield was 70%; ESA-MS: m/z=466.1 [M+H]⁺.

### 5-(3-Cyanophenyl)-N-(3-(3,3,3-trifluoro-2-hydroxy-2-methylpropyl)-1,2,4-thiadiazol-5-yl) furan-3-carboxamide (Compound B-26)

Preparation of compound B-26 referred to the synthesis of compound A-75, using compound B-2 as the starting material to obtain a light yellow solid, yield was 75%; ESA-MS: m/z=423.1 [M+H]⁺.

### 5-(3-Methoxyphenyl)-N-(3-(3,3,3-trifluoro-2-hydroxy-2-methylpropyl)-1,2,4-thiadiazol-5-yl)furan-3-carboxamide (Compound B-27)

Preparation of compound B-27 referred to the synthesis of compound A-75, using compound B-3 as the starting material to obtain a light yellow solid, yield was 73%; ESA-MS: m/z=428.1 [M+H]⁺.

### N-(3-(3,3,3-trifluoro-2-hydroxy-2-methylpropyl)-1,2,4-thiadiazol-5-yl)-5-(3-(trifluoro-methoxy)phenyl)furan-3-carboxamide (Compound B-28)

Preparation of compound B-28 referred to the synthesis of compound A-75, using compound B-4 as the starting material to obtain a light yellow solid, yield was 69%; ESA-MS: m/z=482.1 [M+H]⁺.

### 5-(3-(Difluoromethoxy)phenyl)-N-(3-(3,3,3-trifluoro-2-hydroxy-2-methylpropyl)-1,2,4-thiadiazol-5-yl)furan-3-carboxamide (Compound B-29)

Preparation of compound B-29 referred to the synthesis of compound A-75, using compound B-5 as the starting material to obtain a light yellow solid, yield was 66%; ESA-MS: m/z=464.1 [M+H]⁺.

### Example 23: Synthesis of Compounds B-30˜B-34

The synthesis route is shown in FIG. 27.

### N-(3-(3,3-difluoro-2-methylallyl)-1,2,4-thiadiazol-5-yl)-5-(3-(trifluoromethyl)phenyl) furan-3-carboxamide (Compound B-30)

Preparation of compound B-30 referred to the synthesis of compound A-80, using compound B-1 as the starting material to obtain a light yellow solid, yield was 62%; ESA-MS: m/z=430.1 [M+H]⁺.

### 5-(3-Cyanophenyl)-N-(3-(3,3-difluoro-2-methylallyl)-1,2,4-thiadiazol-5-yl)furan-3-carboxamide (Compound B-31)

Preparation of compound B-31 referred to the synthesis of compound A-80, using compound B-2 as the starting material to obtain a light yellow solid, yield was 65%; ESA-MS: m/z=387.1 [M+H]⁺.

### N-(3-(3,3-difluoro-2-methylallyl)-1,2,4-thiadiazol-5-yl)-5-(3-methoxyphenyl)furan-3-carboxamide (Compound B-32)

Preparation of compound B-32 referred to the synthesis of compound A-80, using compound B-3 as the starting material to obtain a light yellow solid, yield was 69%; ESA-MS: m/z=392.1 [M+H]⁺.

### N-(3-(3,3-difluoro-2-methylallyl)-1,2,4-thiadiazol-5-yl)-5-(3-(trifluoromethoxy)phenyl) furan-3-carboxamide (Compound B-33)

Preparation of compound B-33 referred to the synthesis of compound A-80, using compound B-4 as the starting material to obtain a light yellow solid, yield was 67%; ESA-MS: m/z=446.1 [M+H]⁺.

### N-(3-(3,3-difluoro-2-methylallyl)-1,2,4-thiadiazol-5-yl)-5-(3-(difluoromethoxy)phenyl) furan-3-carboxamide (Compound B-34)

Preparation of compound B-34 referred to the synthesis of compound A-80, using compound B-5 as the starting material to obtain a light yellow solid, yield was 70%; ESA-MS: m/z=428.1 [M+H]⁺.

### Example 24: Synthesis of Compounds B-35˜B-39

The synthesis route is shown in FIG. 28.

### N-(3-(2,2-difluoropropyl)-1,2,4-thiadiazol-5-yl)-5-(3-(trifluoromethyl)phenyl)furan-3-carboxamide (Compound B-35)

Preparation of compound B-35 referred to the synthesis of compound A-85, using compound B-1 as the starting material to obtain a light yellow solid, yield was 58%; ESA-MS: m/z=418.1 [M+H]⁺.

### 5-(3-Cyanophenyl)-N-(3-(2,2-difluoropropyl)-1,2,4-thiadiazol-5-yl)furan-3-carboxamide (Compound B-36)

Preparation of compound B-36 referred to the synthesis of compound A-85, using compound B-2 as the starting material to obtain a light yellow solid, yield was 55%; ESA-MS: m/z=375.1 [M+H]⁺.

### N-(3-(2,2-difluoropropyl)-1,2,4-thiadiazol-5-yl)-5-(3-methoxyphenyl)furan-3-carboxamide (Compound B-37)

Preparation of compound B-37 referred to the synthesis of compound A-85, using compound B-3 as the starting material to obtain a light yellow solid, yield was 63%; ESA-MS: m/z=380.1 [M+H]⁺.

### N-(3-(2,2-difluoropropyl)-1,2,4-thiadiazol-5-yl)-5-(3-(trifluoromethoxy)phenyl)furan-3-carboxamide (Compound B-38)

Preparation of compound B-38 referred to the synthesis of compound A-85, using compound B-4 as the starting material to obtain a light yellow solid, yield was 66%; ESA-MS: m/z=434.1 [M+H]⁺.

### 5-(3-(Difluoromethoxy)phenyl)-N-(3-(2,2-difluoropropyl)-1,2,4-thiadiazol-5-yl)furan-3-carboxamide (Compound B-39)

Preparation of compound B-39 referred to the synthesis of compound A-85, using compound B-5 as the starting material to obtain a light yellow solid, yield was 57%; ESA-MS: m/z=416.1 [M+H]⁺.

### Example 25: Synthesis of Compounds B-40˜B-58

The synthesis route is shown in FIG. 29.

### N-(3-(chloromethyl)-1,2,4-thiadiazol-5-yl)-5-(3-(trifluoromethyl)phenyl)furan-3-carboxamide (Compound B-40)

Preparation of compound B-40 referred to the synthesis of compound A-105, using 5-(3-(trifluoromethyl)phenyl)furan-3-carboxylic acid (compound VII-3-1) as the starting material to obtain a light yellow solid, yield was 31%; ESA-MS: m/z=388.0 [M+H]⁺.

### N-(3-(chloromethyl)-1,2,4-thiadiazol-5-yl)-5-(3-cyanophenyl)furan-3-carboxamide (Compound B-41)

Preparation of compound B-41 referred to the synthesis of compound A-105, using 5-(3-cyanophenyl)furan-3-carboxylic acid (compound VII-3-2) as the starting material to obtain a light yellow solid, yield was 29%; ESA-MS: m/z=345.0 [M+H]⁺.

### N-(3-(chloromethyl)-1,2,4-thiadiazol-5-yl)-5-(3-methoxyphenyl)furan-3-carboxamide (Compound B-42)

Preparation of compound B-42 referred to the synthesis of compound A-105, using 5-(3-methoxyphenyl)furan-3-carboxylic acid (compound VII-3-3) as the starting material to obtain a light yellow solid, yield was 35%; ESA-MS: m/z=350.0 [M+H]⁺.

### N-(3-(chloromethyl)-1,2,4-thiadiazol-5-yl)-5-(3-(trifluoromethoxy)phenyl)furan-3-carboxamide (Compound B-43)

Preparation of compound B-43 referred to the synthesis of compound A-105, using 5-(3-(trifluoromethoxy)phenyl)furan-3-carboxylic acid (compound VII-3-4) as the starting material to obtain a light yellow solid, yield was 38%; ESA-MS: m/z=404.0 [M+H]⁺.

### N-(3-(chloromethyl)-1,2,4-thiadiazol-5-yl)-5-(3-(difluoromethoxy)phenyl)furan-3-carboxamide (Compound B-44)

Preparation of compound B-44 referred to the synthesis of compound A-105, using 5-(3-(difluoromethoxy)phenyl)furan-3-carboxylic acid (compound VII-3-5) as the starting material to obtain a light yellow solid, yield was 29%; ESA-MS: m/z=386.0 [M+H]⁺.

### N-(3-(morpholinomethyl)-1,2,4-thiadiazol-5-yl)-5-(3-(trifluoromethyl)phenyl)furan-3-carboxamide (Compound B-45)

Preparation of compound B-45 referred to the synthesis of compound A-106, using compound B-40 and morpholine as the starting material to obtain a light yellow solid, yield was 79%; ESA-MS: m/z=439.1 [M+H]⁺.

### N-(3-((4-methylpiperazin-1-yl)methyl)-1,2,4-thiadiazol-5-yl)-5-(3-(trifluoromethyl)phenyl)furan-3-carboxamide (Compound B-46)

Preparation of compound B-46 referred to the synthesis of compound A-106, using compound B-40 and 1-methylpiperazine as the starting material to obtain a light yellow solid, yield was 78%; ESA-MS: m/z=452.1 [M+H]⁺.

### 5-(3-Cyanophenyl)-N-(3-((dimethylamino)methyl)-1,2,4-thiadiazol-5-yl)furan-3-carboxamide (Compound B-47)

Preparation of compound B-47 referred to the synthesis of compound A-106, using compound B-41 and dimethylamine hydrochloride as the starting material to obtain a light yellow solid, yield was 68%; ESA-MS: m/z=354.1 [M+H]⁺.

### 5-(3-Cyanophenyl)-N-(3-(morpholinomethyl)-1,2,4-thiadiazol-5-yl)furan-3-carboxamide (Compound B-48)

Preparation of compound B-48 referred to the synthesis of compound A-106, using compound B-41 and morpholine as the starting material to obtain a light yellow solid, yield was 72%; ESA-MS: m/z=396.1 [M+H]⁺.

### 5-(3-Cyanophenyl)-N-(3-((4-methylpiperazin-1-yl)methyl)-1,2,4-thiadiazol-5-yl)furan-3-carboxamide (Compound B-49)

Preparation of compound B-49 referred to the synthesis of compound A-106, using compound B-41 and 1-methylpiperazine as the starting material to obtain a light yellow solid, yield was 74%; ESA-MS: m/z=409.1 [M+H]⁺.

### N-(3-((diethylamino)methyl)-1,2,4-thiadiazol-5-yl)-5-(3-methoxyphenyl)furan-3-carboxamide (Compound B-50)

Preparation of compound B-50 referred to the synthesis of compound A-106, using compound B-42 and dicthylamine hydrochloride as the starting material to obtain a light yellow solid, yield was 65%; ESA-MS: m/z=387.1 [M+H]⁺.

### 5-(3-Methoxyphenyl)-N-(3-(morpholinomethyl)-1,2,4-thiadiazol-5-yl)furan-3-carboxamide (Compound B-51)

Preparation of compound B-51 referred to the synthesis of compound A-106, using compound B-42 and morpholine as the starting material to obtain a light yellow solid, yield was 69%; ESA-MS: m/z=401.1 [M+H]⁺.

### 5-(3-Methoxyphenyl)-N-(3-((4-methylpiperazin-1-yl)methyl)-1,2,4-thiadiazol-5-yl)furan-3-carboxamide (Compound B-52)

Preparation of compound B-52 referred to the synthesis of compound A-106, using compound B-42 and 1-methylpiperazine as the starting material to obtain a light yellow solid, yield was 77%; ESA-MS: m/z=414.2 [M+H]⁺.

### N-(3-(pyrrolidin-1-ylmethyl)-1,2,4-thiadiazol-5-yl)-5-(3-(trifluoromethoxy)phenyl)furan-3-carboxamide (Compound B-53)

Preparation of compound B-53 referred to the synthesis of compound A-106, using compound B-43 and pyrrolidine as the starting material to obtain a light yellow solid, yield was 72%; ESA-MS: m/z=439.1 [M+H]⁺.

### N-(3-(morpholinomethyl)-1,2,4-thiadiazol-5-yl)-5-(3-(trifluoromethoxy)phenyl)furan-3-carboxamide (Compound B-54)

Preparation of compound B-54 referred to the synthesis of compound A-106, using compound B-43 and morpholine as the starting material to obtain a light yellow solid, yield was 68%; ESA-MS: m/z=455.1 [M+H]⁺.

### N-(3-((4-methylpiperazin-1-yl)methyl)-1,2,4-thiadiazol-5-yl)-5-(3-(trifluoromethoxy)phenyl)furan-3-carboxamide (Compound B-55)

Preparation of compound B-55 referred to the synthesis of compound A-106, using compound B-43 and 1-methylpiperazine as the starting material to obtain a light yellow solid, yield was 74%; ESA-MS: m/z=468.1 [M+H]⁺.

### 5-(3-(Difluoromethoxy)phenyl)-N-(3-(piperidin-1-ylmethyl)-1,2,4-thiadiazol-5-yl)furan-3-carboxamide (Compound B-56)

Preparation of compound B-56 referred to the synthesis of compound A-106, using compound B-44 and piperidine as the starting material to obtain a light yellow solid, yield was 68%; ESA-MS: m/z=435.1 [M+H]⁺.

### 5-(3-(Difluoromethoxy)phenyl)-N-(3-(morpholinomethyl)-1,2,4-thiadiazol-5-yl)furan-3-carboxamide (Compound B-57)

Preparation of compound B-57 referred to the synthesis of compound A-106, using compound B-44 and morpholine as the starting material to obtain a light yellow solid, yield was 69%; ESA-MS: m/z=437.1 [M+H]⁺.

### 5-(3-(Difluoromethoxy)phenyl)-N-(3-((4-methylpiperazin-1-yl)methyl)-1,2,4-thiadiazol-5-yl)furan-3-carboxamide (Compound B-58)

Preparation of compound B-58 referred to the synthesis of compound A-106, using compound B-44 and 1-methylpiperazine as the starting material to obtain a light yellow solid, yield was 71%; ESA-MS: m/z=450.1 [M+H]⁺.

### Example 26: Synthesis of Compounds C-1˜C-5

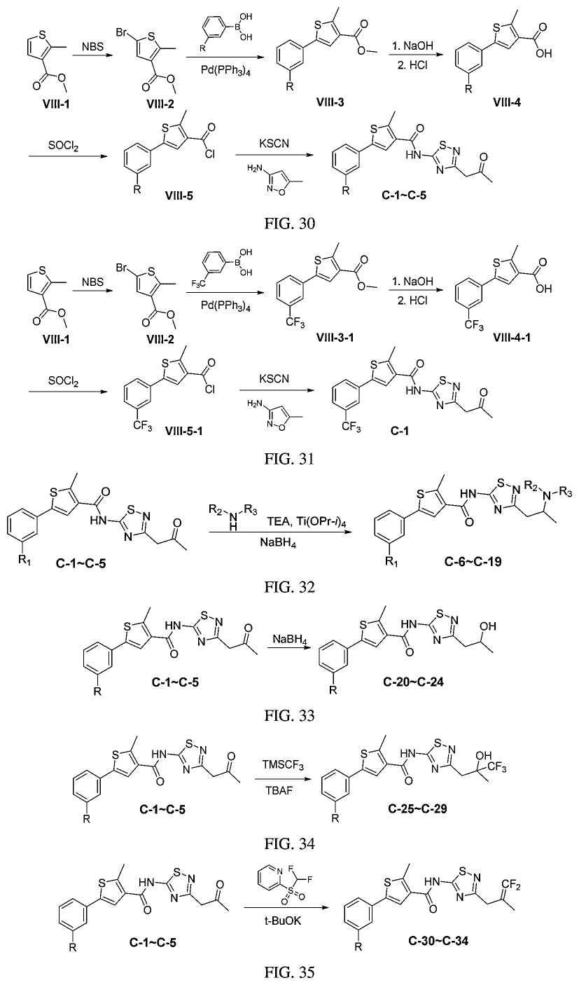

The synthesis route is shown in FIG. 30.

### 2-Methyl-N-(3-(2-oxopropyl)-1,2,4-thiadiazol-5-yl)-5-(3-(trifluoromethyl)phenyl)thiophene-3-carboxamide (Compound C-1)

The specific synthesis route is shown in FIG. 31.

### (a) Synthesis of methyl 5-bromo-2-methylthiophene-3-carboxylate (Compound VIII-2)

To a solution of methyl 2-methylthiophene-3-carboxylate (VIII-1, 1.00 g, 6.4 mmol) in DMF (8 mL) was added NBS (1.14 g, 6.4 mmol) and the mixture was stirred at room temperature overnight. Ethyl acetate was added to the reaction mixture and the organic layer was washed with water and brine, dried over anhydrous Na₂SO₄, and concentrated to give a residue, which was purified by column-chromatograph to give a yellow oil, yield was 85%; ESA-MS: m/z=234.9 [M+H]⁺.

### (b) Synthesis of methyl 2-methyl-5-(3-(trifluoromethyl)phenyl)thiophene-3-carboxylate (Compound VIII-3-1)

To a solution of compound VIII-2 (1.20 g, 5.1 mmol) in DME (10 mL) was added 3-(trifluoromethyl)phenylboronic acid (1.21 g, 6.4 mmol), tetrakis(triphenylphosphine)palladium (295 mg, 0.26 mmol) and potassium carbonate (2.12 g, 15.3 mmol) and the reaction mixture was stirred at 85° C. for 2 hours. After removing the solvent under reduced pressure, ethyl acetate was added to the reaction mixture and the organic layer was washed with water and brine, dried over anhydrous Na₂SO₄, and concentrated to give a residue, which was purified by column-chromatograph to give a light yellow solid, yield was 69%; ESA-MS: m/z=301.0 [M+H]⁺.

### (c) Synthesis of 2-methyl-5-(3-(trifluoromethyl)phenyl)thiophene-3-carboxylic acid (Compound VIII-4-1)

To a solution of compound VIII-3-1 (1.05 g, 3.5 mmol) in ethanol and water (3:1, 20 mL) was added sodium hydroxide (840 mg, 21.0 mmol) and stirred overnight at room temperature. The reaction mixture was adjusted with 2 mol/L HCl to acidity, and ethyl acetate was added. The mixture was washed with water and brine, dried over anhydrous Na₂SO₄, and concentrated to give a light yellow solid, yield was 89%; ESA-MS: m/z=287.0 [M+H]⁺.

### (d) Synthesis of 2-methyl-5-(3-(trifluoromethyl)phenyl)thiophene-3-carbonyl chloride (Compound VIII-5-1)

A solution of compound VIII-4-1 (890 mg, 3.1 mmol) in 10 mL SOCl₂ was heated to reflux for 2.5 hours, and the mixture was concentrated to dryness to get a light yellow solid, yield was 99%;

### (c) Synthesis of 2-methyl-N-(3-(2-oxopropyl)-1,2,4-thiadiazol-5-yl)-5-(3-(trifluoromethyl)phenyl)thiophene-3-carboxamide (Compound C-1)

A suspension of compound VIII-5-1 (940 mg, 3.1 mmol) and potassium thiocyanate (330 mg, 3.4 mmol) in 15 mL acetonitrile was stirred at 60° C. for 30 min, and then 5-methylisoxazol-3-amine (303 mg, 3.1 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 62%; ¹H NMR (500 MHZ, Chloroform-d): δ 7.80 (t, J=1.5 Hz, 1H), 7.76-7.72 (m, 1H), 7.65 (m, 2H), 7.58 (t, J=7.5 Hz, 1H), 3.58 (s, 2H), 2.45 (s, 3H), 2.30 (s, 3H); ESA-MS: m/z=426.0 [M+H]⁺.

### 5-(3-Cyanophenyl)-2-methyl-N-(3-(2-oxopropyl)-1,2,4-thiadiazol-5-yl)thiophene-3-carboxamide (Compound C-2)

Preparation of compound C-2 referred to the synthesis of compound C-1, replacing (3-(trifluoromethyl)phenyl) boronic acid with (3-cyanophenyl) boronic acid to obtain a light yellow solid, yield was 31%; ESA-MS: m/z=383.1 [M+H]⁺.

### 5-(3-Methoxyphenyl)-2-methyl-N-(3-(2-oxopropyl)-1,2,4-thiadiazol-5-yl)thiophene-3-carboxamide (Compound C-3)

Preparation of compound C-3 referred to the synthesis of compound C-1, replacing (3-(trifluoromethyl)phenyl) boronic acid with (3-methoxyphenyl) boronic acid to obtain a light yellow solid, yield was 33%; ESA-MS: m/z=388.1 [M+H]⁺.

### 2-Methyl-N-(3-(2-oxopropyl)-1,2,4-thiadiazol-5-yl)-5-(3-(trifluoromethoxy)phenyl)thiophene-3-carboxamide (Compound C-4)

Preparation of compound C-4 referred to the synthesis of compound C-1, replacing (3-(trifluoromethyl)phenyl) boronic acid with (3-(trifluoromethoxy)phenyl) boronic acid to obtain a light yellow solid, yield was 28%; ESA-MS: m/z=442.0 [M+H]⁺.

### 5-(3-(Difluoromethoxy)phenyl)-2-methyl-N-(3-(2-oxopropyl)-1,2,4-thiadiazol-5-yl)thiophene-3-carboxamide (Compound C-5)

Preparation of compound C-5 referred to the synthesis of compound C-1, replacing (3-(trifluoromethyl)phenyl) boronic acid with (3-(difluoromethoxy)phenyl) boronic acid to obtain a light yellow solid, yield was 25%; ESA-MS: m/z=424.1 [M+H]⁺.

### Example 27: Synthesis of Compounds C-6˜C-19

The synthesis route is shown in FIG. 32.

### N-(3-(2-(dimethylamino)propyl)-1,2,4-thiadiazol-5-yl)-2-methyl-5-(3-(trifluoromethyl)phenyl)thiophene-3-carboxamide (Compound C-6)

Preparation of compound C-6 referred to the synthesis of compound A-38, using compound C-1 and dimethylamine hydrochloride as the starting material to obtain a light yellow solid, yield was 75%; ESA-MS: m/z=455.1 [M+H]⁺.

### N-(3-(2-(diethylamino)propyl)-1,2,4-thiadiazol-5-yl)-2-methyl-5-(3-(trifluoromethyl)phenyl)thiophene-3-carboxamide (Compound C-7)

Preparation of compound C-7 referred to the synthesis of compound A-38, using compound C-1 and diethylamine hydrochloride as the starting material to obtain a light yellow solid, yield was 76%; ESA-MS: m/z=483.1 [M+H]⁺.

### 2-Methyl-N-(3-(2-(pyrrolidin-1-yl)propyl)-1,2,4-thiadiazol-5-yl)-5-(3-(trifluoromethyl)phenyl)thiophene-3-carboxamide (Compound C-8)

Preparation of compound C-8 referred to the synthesis of compound A-38, using compound C-1 and pyrrolidine as the starting material to obtain a light yellow solid, yield was 66%; ESA-MS: m/z=481.1 [M+H]⁺.

### 2-Methyl-N-(3-(2-(piperidin-1-yl)propyl)-1,2,4-thiadiazol-5-yl)-5-(3-(trifluoromethyl)phenyl)thiophene-3-carboxamide (Compound C-9)

Preparation of compound C-9 referred to the synthesis of compound A-38, using compound C-1 and piperidine as the starting material to obtain a light yellow solid, yield was 70%; ESA-MS: m/z=495.1 [M+H]⁺.

### 2-Methyl-N-(3-(2-morpholinopropyl)-1,2,4-thiadiazol-5-yl)-5-(3-(trifluoromethyl)phenyl)thiophene-3-carboxamide (Compound C-10)

Preparation of compound C-10 referred to the synthesis of compound A-38, using compound C-1 and morpholine as the starting material to obtain a light yellow solid, yield was 67%; ESA-MS: m/z=497.1 [M+H]⁺.

### 2-Methyl-N-(3-(2-(4-methylpiperazin-1-yl)propyl)-1,2,4-thiadiazol-5-yl)-5-(3-(trifluoro-methyl)phenyl)thiophene-3-carboxamide (Compound C-11)

Preparation of compound C-11 referred to the synthesis of compound A-38, using compound C-1 and 1-methylpiperazine as the starting material to obtain a light yellow solid, yield was 69%; ESA-MS: m/z=510.2 [M+H]⁺.

### 5-(3-Cyanophenyl)-2-methyl-N-(3-(2-(piperidin-1-yl)propyl)-1,2,4-thiadiazol-5-yl)thiophene-3-carboxamide (Compound C-12)

Preparation of compound C-12 referred to the synthesis of compound A-38, using compound C-2 and piperidine as the starting material to obtain a light yellow solid, yield was 73%; ESA-MS: m/z=452.2 [M+H]⁺.

### 5-(3-Cyanophenyl)-2-methyl-N-(3-(2-morpholinopropyl)-1,2,4-thiadiazol-5-yl)thiophene-3-carboxamide (Compound C-13)

Preparation of compound C-13 referred to the synthesis of compound A-38, using compound C-2 and morpholine as the starting material to obtain a light yellow solid, yield was 68%; ESA-MS: m/z=454.1 [M+H]⁺.

### 5-(3-Methoxyphenyl)-2-methyl-N-(3-(2-(pyrrolidin-1-yl)propyl)-1,2,4-thiadiazol-5-yl)thiophene-3-carboxamide (Compound C-14)

Preparation of compound C-14 referred to the synthesis of compound A-38, using compound C-3 and pyrrolidine as the starting material to obtain a light yellow solid, yield was 71%; ESA-MS: m/z=443.2 [M+H]⁺.

### 5-(3-Methoxyphenyl)-2-methyl-N-(3-(2-morpholinopropyl)-1,2,4-thiadiazol-5-yl)thiophene-3-carboxamide (Compound C-15)

Preparation of compound C-15 referred to the synthesis of compound A-38, using compound C-3 and morpholine as the starting material to obtain a light yellow solid, yield was 75%; ESA-MS: m/z=459.1 [M+H]⁺.

### 5-(3-Methoxyphenyl)-2-methyl-N-(3-(2-(4-methylpiperazin-1-yl)propyl)-1,2,4-thiadiazol-5-yl)thiophene-3-carboxamide (Compound C-16)

Preparation of compound C-16 referred to the synthesis of compound A-38, using compound C-3 and 1-methylpiperazine as the starting material to obtain a light yellow solid, yield was 74%; ESA-MS: m/z=472.2 [M+H]⁺.

### 2-Methyl-N-(3-(2-morpholinopropyl)-1,2,4-thiadiazol-5-yl)-5-(3-(trifluoromethoxy)phenyl)thiophene-3-carboxamide (Compound C-17)

Preparation of compound C-17 referred to the synthesis of compound A-38, using compound C-4 and morpholine as the starting material to obtain a light yellow solid, yield was 72%; ESA-MS: m/z=513.1 [M+H]⁺.

### 5-(3-(Difluoromethoxy)phenyl)-2-methyl-N-(3-(2-morpholinopropyl)-1,2,4-thiadiazol-5-yl)thiophene-3-carboxamide (Compound C-18)

Preparation of compound C-18 referred to the synthesis of compound A-38, using compound C-5 and morpholine as the starting material to obtain a light yellow solid, yield was 70%; ESA-MS: m/z=495.1 [M+H]⁺.

### 5-(3-(Difluoromethoxy)phenyl)-2-methyl-N-(3-(2-(4-methylpiperazin-1-yl)propyl)-1,2,4-thiadiazol-5-yl)thiophene-3-carboxamide (Compound C-19)

Preparation of compound C-19 referred to the synthesis of compound A-38, using compound C-5 and 1-methylpiperazine as the starting material to obtain a light yellow solid, yield was 68%; ESA-MS: m/z=508.2 [M+H]⁺.

### Example 28: Synthesis of Compounds C-20˜C-24

The synthesis route is shown in FIG. 33.

### N-(3-(2-hydroxypropyl)-1,2,4-thiadiazol-5-yl)-2-methyl-5-(3-(trifluoromethyl)phenyl)thiophene-3-carboxamide (Compound C-20)

Preparation of compound C-20 referred to the synthesis of compound A-60, using compound C-1 as the starting material to obtain a light yellow solid, yield was 81%; ESA-MS: m/z=428.1 [M+H]⁺.

### 5-(3-Cyanophenyl)-N-(3-(2-hydroxypropyl)-1,2,4-thiadiazol-5-yl)-2-methylthiophene-3-carboxamide (Compound C-21)

Preparation of compound C-21 referred to the synthesis of compound A-60, using compound C-2 as the starting material to obtain a light yellow solid, yield was 85%; ESA-MS: m/z=385.1 [M+H]⁺.

### N-(3-(2-hydroxypropyl)-1,2,4-thiadiazol-5-yl)-5-(3-methoxyphenyl)-2-methylthiophene-3-carboxamide (Compound C-22)

Preparation of compound C-22 referred to the synthesis of compound A-60, using compound C-3 as the starting material to obtain a light yellow solid, yield was 77%; ESA-MS: m/z=390.1 [M+H]⁺.

### N-(3-(2-hydroxypropyl)-1,2,4-thiadiazol-5-yl)-2-methyl-5-(3-(trifluoromethoxy)phenyl)thiophene-3-carboxamide (Compound C-23)

Preparation of compound C-23 referred to the synthesis of compound A-60, using compound C-4 as the starting material to obtain a light yellow solid, yield was 79%; ESA-MS: m/z=444.1 [M+H]⁺.

### 5-(3-(Difluoromethoxy)phenyl)-N-(3-(2-hydroxypropyl)-1,2,4-thiadiazol-5-yl)-2-methyl-thiophene-3-carboxamide (Compound C-24)

Preparation of compound C-24 referred to the synthesis of compound A-60, using compound C-5 as the starting material to obtain a light yellow solid, yield was 85%; ESA-MS: m/z=426.1 [M+H]⁺.

### Example 29: Synthesis of Compounds C-25˜C-29

The synthesis route is shown in FIG. 34.

### 2-Methyl-N-(3-(3,3,3-trifluoro-2-hydroxy-2-methylpropyl)-1,2,4-thiadiazol-5-yl)-5-(3-(trifluoromethyl)phenyl)thiophene-3-carboxamide (Compound C-25)

Preparation of compound C-25 referred to the synthesis of compound A-75, using compound C-1 as the starting material to obtain a light yellow solid, yield was 72%; ESA-MS: m/z=496.1 [M+H]⁺.

### 5-(3-Cyanophenyl)-2-methyl-N-(3-(3,3,3-trifluoro-2-hydroxy-2-methylpropyl)-1,2,4-thiadiazol-5-yl)thiophene-3-carboxamide (Compound C-26)

Preparation of compound C-26 referred to the synthesis of compound A-75, using compound C-2 as the starting material to obtain a light yellow solid, yield was 71%; ESA-MS: m/z=453.1 [M+H]⁺.

### 5-(3-Methoxyphenyl)-2-methyl-N-(3-(3,3,3-trifluoro-2-hydroxy-2-methylpropyl)-1,2,4-thiadiazol-5-yl)thiophene-3-carboxamide (Compound C-27)

Preparation of compound C-27 referred to the synthesis of compound A-75, using compound C-3 as the starting material to obtain a light yellow solid, yield was 63%; ESA-MS: m/z=458.1 [M+H]⁺.

### 2-Methyl-N-(3-(3,3,3-trifluoro-2-hydroxy-2-methylpropyl)-1,2,4-thiadiazol-5-yl)-5-(3-(trifluoromethoxy)phenyl)thiophene-3-carboxamide (Compound C-28)

Preparation of compound C-28 referred to the synthesis of compound A-75, using compound C-4 as the starting material to obtain a light yellow solid, yield was 65%; ESA-MS: m/z=512.0 [M+H]⁺.

### 5-(3-(Difluoromethoxy)phenyl)-2-methyl-N-(3-(3,3,3-trifluoro-2-hydroxy-2-methylpropyl)-1,2,4-thiadiazol-5-yl)thiophene-3-carboxamide (Compound C-29)

Preparation of compound C-29 referred to the synthesis of compound A-75, using compound C-5 as the starting material to obtain a light yellow solid, yield was 76%; ESA-MS: m/z=494.1 [M+H]⁺.

### Example 30: Synthesis of Compounds C-30˜C-34

The synthesis route is shown in FIG. 35.

### N-(3-(3,3-difluoro-2-methylallyl)-1,2,4-thiadiazol-5-yl)-2-methyl-5-(3-(trifluoromethyl)phenyl)thiophene-3-carboxamide (Compound C-30)

Preparation of compound C-30 referred to the synthesis of compound A-80, using compound C-1 as the starting material to obtain a light yellow solid, yield was 65%; ESA-MS: m/z=460.0 [M+H]⁺.

### 5-(3-Cyanophenyl)-N-(3-(3,3-difluoro-2-methylallyl)-1,2,4-thiadiazol-5-yl)-2-methyl-thiophene-3-carboxamide (Compound C-31)

Preparation of compound C-31 referred to the synthesis of compound A-80, using compound C-2 as the starting material to obtain a light yellow solid, yield was 60%; ESA-MS: m/z=417.1 [M+H]⁺.

### N-(3-(3,3-difluoro-2-methylallyl)-1,2,4-thiadiazol-5-yl)-5-(3-methoxyphenyl)-2-methyl-thiophene-3-carboxamide (Compound C-32)

Preparation of compound C-32 referred to the synthesis of compound A-80, using compound C-3 as the starting material to obtain a light yellow solid, yield was 59%; ESA-MS: m/z=422.1 [M+H]⁺.

### N-(3-(3,3-difluoro-2-methylallyl)-1,2,4-thiadiazol-5-yl)-2-methyl-5-(3-(trifluoromethoxy)phenyl)thiophene-3-carboxamide (Compound C-33)

Preparation of compound C-33 referred to the synthesis of compound A-80, using compound C-4 as the starting material to obtain a light yellow solid, yield was 57%; ESA-MS: m/z=476.0 [M+H]⁺.

### N-(3-(3,3-difluoro-2-methylallyl)-1,2,4-thiadiazol-5-yl)-5-(3-(difluoromethoxy)phenyl)-2-methylthiophene-3-carboxamide (Compound C-34)

Preparation of compound C-34 referred to the synthesis of compound A-80, using compound C-5 as the starting material to obtain a light yellow solid, yield was 64%; ESA-MS: m/z=458.1 [M+H]⁺.

### Example 31: Synthesis of Compounds C-35˜C-39

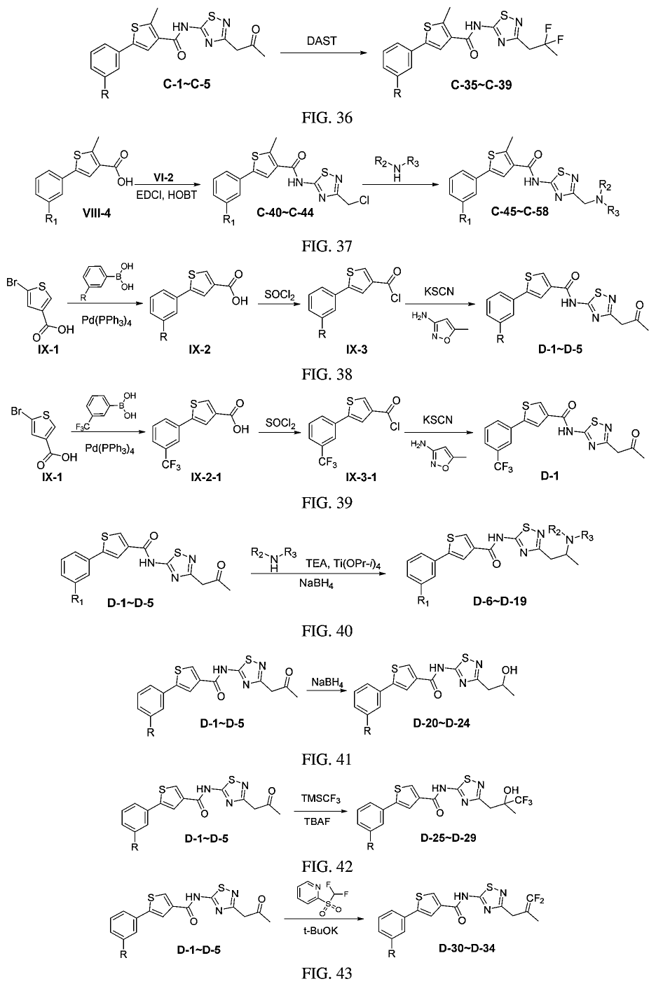

The synthesis route is shown in FIG. 36.

### N-(3-(2,2-difluoropropyl)-1,2,4-thiadiazol-5-yl)-2-methyl-5-(3-(trifluoromethyl)phenyl)thiophene-3-carboxamide (Compound C-35)

Preparation of compound C-35 referred to the synthesis of compound A-85, using compound C-1 as the starting material to obtain a light yellow solid, yield was 58%; ESA-MS: m/z=448.0 [M+H]⁺.

### 5-(3-Cyanophenyl)-N-(3-(2,2-difluoropropyl)-1,2,4-thiadiazol-5-yl)-2-methylthiophene-3-carboxamide (Compound C-36)

Preparation of compound C-36 referred to the synthesis of compound A-85, using compound C-2 as the starting material to obtain a light yellow solid, yield was 55%; ESA-MS: m/z=405.1 [M+H]⁺.

### N-(3-(2,2-difluoropropyl)-1,2,4-thiadiazol-5-yl)-5-(3-methoxyphenyl)-2-methylthiophene-3-carboxamide (Compound C-37)

Preparation of compound C-37 referred to the synthesis of compound A-85, using compound C-3 as the starting material to obtain a light yellow solid, yield was 63%; ESA-MS: m/z=410.1 [M+H]⁺.

### N-(3-(2,2-difluoropropyl)-1,2,4-thiadiazol-5-yl)-2-methyl-5-(3-(trifluoromethoxy)phenyl)thiophene-3-carboxamide (Compound C-38)

Preparation of compound C-38 referred to the synthesis of compound A-85, using compound C-4 as the starting material to obtain a light yellow solid, yield was 66%; ESA-MS: m/z=464.0 [M+H]⁺.

### 5-(3-(Difluoromethoxy)phenyl)-N-(3-(2,2-difluoropropyl)-1,2,4-thiadiazol-5-yl)-2-methyl-thiophene-3-carboxamide (Compound C-39)

Preparation of compound C-39 referred to the synthesis of compound A-85, using compound C-5 as the starting material to obtain a light yellow solid, yield was 57%; ESA-MS: m/z=446.1 [M+H]⁺.

### Example 32: Synthesis of Compounds C-40˜C-58

The synthesis route is shown in FIG. 37.

### N-(3-(chloromethyl)-1,2,4-thiadiazol-5-yl)-2-methyl-5-(3-(trifluoromethyl)phenyl)thiophene-3-carboxamide (Compound C-40)

Preparation of compound C-40 referred to the synthesis of compound A-105, using 2-methyl-5-(3-(trifluoromethyl)phenyl)thiophene-3-carboxylic acid (compound VIII-4-1) as the starting material to obtain a light yellow solid, yield was 32%; ESA-MS: m/z=418.0 [M+H]⁺.

### N-(3-(chloromethyl)-1,2,4-thiadiazol-5-yl)-5-(3-cyanophenyl)-2-methylthiophene-3-carboxamide (Compound C-41)

Preparation of compound C-41 referred to the synthesis of compound A-105, using 5-(3-cyanophenyl)-2-methylthiophene-3-carboxylic acid (compound VIII-4-2) as the starting material to obtain a light yellow solid, yield was 30%; ESA-MS: m/z=375.0 [M+H]⁺.

### N-(3-(chloromethyl)-1,2,4-thiadiazol-5-yl)-5-(3-methoxyphenyl)-2-methylthiophene-3-carboxamide (Compound C-42)

Preparation of compound C-42 referred to the synthesis of compound A-105, using 5-(3-methoxyphenyl)-2-methylthiophene-3-carboxylic acid (compound VIII-4-3) as the starting material to obtain a light yellow solid, yield was 32%; ESA-MS: m/z=380.0 [M+H]⁺.

### N-(3-(chloromethyl)-1,2,4-thiadiazol-5-yl)-2-methyl-5-(3-(trifluoromethoxy)phenyl)thiophene-3-carboxamide (Compound C-43)

Preparation of compound C-43 referred to the synthesis of compound A-105, using 2-methyl-5-(3-(trifluoromethoxy)phenyl)thiophene-3-carboxylic acid (compound VIII-4-4) as the starting material to obtain a light yellow solid, yield was 35%; ESA-MS: m/z=434.0 [M+H]⁺.

### N-(3-(chloromethyl)-1,2,4-thiadiazol-5-yl)-5-(3-(difluoromethoxy)phenyl)-2-methyl-thiophene-3-carboxamide (Compound C-44)

Preparation of compound C-44 referred to the synthesis of compound A-105, using 2-methyl-5-(3-(difluoromethoxy)phenyl)thiophene-3-carboxylic acid (compound VIII-4-5) as the starting material to obtain a light yellow solid, yield was 31%; ESA-MS: m/z=416.0 [M+H]⁺.

### 2-Methyl-N-(3-(morpholinomethyl)-1,2,4-thiadiazol-5-yl)-5-(3-(trifluoromethyl)phenyl)thiophene-3-carboxamide (Compound C-45)

Preparation of compound C-45 referred to the synthesis of compound A-106, using compound C-40 and morpholine as the starting material to obtain a light yellow solid, yield was 77%; ESA-MS: m/z=469.1 [M+H]⁺.

### 2-Methyl-N-(3-((4-methylpiperazin-1-yl)methyl)-1,2,4-thiadiazol-5-yl)-5-(3-(trifluoro-methyl)phenyl)thiophene-3-carboxamide (Compound C-46)

Preparation of compound C-46 referred to the synthesis of compound A-106, using compound C-40 and 1-methylpiperazine as the starting material to obtain a light yellow solid, yield was 75%; ESA-MS: m/z=482.1 [M+H]⁺.

### 5-(3-Cyanophenyl)-N-(3-((diethylamino)methyl)-1,2,4-thiadiazol-5-yl)-2-methyl-thiophene-3-carboxamide (Compound C-47)

Preparation of compound C-47 referred to the synthesis of compound A-106, using compound C-41 and diethylamine hydrochloride as the starting material to obtain a light yellow solid, yield was 69%; ESA-MS: m/z=412.1 [M+H]⁺.

### 5-(3-Cyanophenyl)-2-methyl-N-(3-(morpholinomethyl)-1,2,4-thiadiazol-5-yl)thiophene-3-carboxamide (Compound C-48)

Preparation of compound C-48 referred to the synthesis of compound A-106, using compound C-41 and morpholine as the starting material to obtain a light yellow solid, yield was 71%; ESA-MS: m/z=426.1 [M+H]⁺.

### 5-(3-Cyanophenyl)-2-methyl-N-(3-((4-methylpiperazin-1-yl)methyl)-1,2,4-thiadiazol-5-yl)thiophene-3-carboxamide (Compound C-49)

Preparation of compound C-49 referred to the synthesis of compound A-106, using compound C-41 and 1-methylpiperazine as the starting material to obtain a light yellow solid, yield was 72%; ESA-MS: m/z=439.1 [M+H]⁺.

### N-(3-((diethylamino)methyl)-1,2,4-thiadiazol-5-yl)-5-(3-methoxyphenyl)-2-methyl-thiophene-3-carboxamide (Compound C-50)

Preparation of compound C-50 referred to the synthesis of compound A-106, using compound C-42 and dimethylamine hydrochloride as the starting material to obtain a light yellow solid, yield was 69%; ESA-MS: m/z=389.1 [M+H]⁺.

### 5-(3-Methoxyphenyl)-2-methyl-N-(3-(morpholinomethyl)-1,2,4-thiadiazol-5-yl)thiophene-3-carboxamide (Compound C-51)

Preparation of compound C-51 referred to the synthesis of compound A-106, using compound C-42 and morpholine as the starting material to obtain a light yellow solid, yield was 63%; ESA-MS: m/z=431.1 [M+H]⁺.

### 5-(3-Methoxyphenyl)-2-methyl-N-(3-((4-methylpiperazin-1-yl)methyl)-1,2,4-thiadiazol-5-yl)thiophene-3-carboxamide (Compound C-52)

Preparation of compound C-52 referred to the synthesis of compound A-106, using compound C-42 and 1-methylpiperazine as the starting material to obtain a light yellow solid, yield was 72%; ESA-MS: m/z=444.1 [M+H]⁺.

### 2-Methyl-N-(3-(pyrrolidin-1-ylmethyl)-1,2,4-thiadiazol-5-yl)-5-(3-(trifluoromethoxy)phenyl)thiophene-3-carboxamide (Compound C-53)

Preparation of compound C-53 referred to the synthesis of compound A-106, using compound C-43 and piperidine as the starting material to obtain a light yellow solid, yield was 77%; ESA-MS: m/z=483.1 [M+H]⁺.

### 2-Methyl-N-(3-(morpholinomethyl)-1,2,4-thiadiazol-5-yl)-5-(3-(trifluoromethoxy)phenyl)thiophene-3-carboxamide (Compound C-54)

Preparation of compound C-54 referred to the synthesis of compound A-106, using compound C-43 and morpholine as the starting material to obtain a light yellow solid, yield was 67%; ESA-MS: m/z=485.1 [M+H]⁺.

### 2-Methyl-N-(3-((4-methylpiperazin-1-yl)methyl)-1,2,4-thiadiazol-5-yl)-5-(3-(trifluoro-methoxy)phenyl)thiophene-3-carboxamide (Compound C-55)

Preparation of compound C-55 referred to the synthesis of compound A-106, using compound C-43 and 1-methylpiperazine as the starting material to obtain a light yellow solid, yield was 73%; ESA-MS: m/z=498.1 [M+H]⁺.

### 5-(3-(Difluoromethoxy)phenyl)-2-methyl-N-(3-(piperidin-1-ylmethyl)-1,2,4-thiadiazol-5-yl)thiophene-3-carboxamide (Compound C-56)

Preparation of compound C-56 referred to the synthesis of compound A-106, using compound C-44 and pyrrolidine as the starting material to obtain a light yellow solid, yield was 64%; ESA-MS: m/z=451.1 [M+H]⁺.

### 5-(3-(Difluoromethoxy)phenyl)-2-methyl-N-(3-(morpholinomethyl)-1,2,4-thiadiazol-5-yl)thiophene-3-carboxamide (Compound C-57)

Preparation of compound C-57 referred to the synthesis of compound A-106, using compound C-44 and morpholine as the starting material to obtain a light yellow solid, yield was 65%; ESA-MS: m/z=467.1 [M+H]⁺.

### 5-(3-(Difluoromethoxy)phenyl)-2-methyl-N-(3-((4-methylpiperazin-1-yl)methyl)-1,2,4-thiadiazol-5-yl)thiophene-3-carboxamide (Compound C-58)

Preparation of compound C-58 referred to the synthesis of compound A-106, using compound C-44 and 1-methylpiperazine as the starting material to obtain a light yellow solid, yield was 75%; ESA-MS: m/z=480.1 [M+H]⁺.

### Example 33: Synthesis of Compounds D-1˜D-5

The synthesis route is shown in FIG. 38.

### N-(3-(2-oxopropyl)-1,2,4-thiadiazol-5-yl)-5-(3-(trifluoromethyl)phenyl)thiophene-3-carboxamide (Compound D-1)

The specific synthesis route is shown in FIG. 39.

### (a) Synthesis of 5-(3-(trifluoromethyl)phenyl)thiophene-3-carboxylic acid (Compound IX-2-1)

To a solution of 5-bromothiophene-3-carboxylic acid (IX-1, 500 mg, 2.4 mmol) in DME (10 mL) was added 3-(trifluoromethyl)phenylboronic acid (505 mg, 2.7 mmol), tetrakis(triphenylphosphine)palladium (419 mg, 0.3 mmol) and sodium bicarbonate (609 mg, 7.2 mmol) under nitrogen atmosphere and the reaction mixture was heated to reflux overnight. After removing the solvent under reduced pressure, ethyl acetate was added to the reaction mixture and the organic layer was washed with water and brine, dried over anhydrous Na₂SO₄, and concentrated to give a residue, which was purified by column-chromatograph to give a light yellow solid, yield was 61%; ESA-MS: m/z=273.0 [M+H]⁺.

### (b) Synthesis of 5-(3-(trifluoromethyl)phenyl)thiophene-3-carbonyl chloride (Compound IX-3-1)

A solution of compound IX-2-1 (390 mg, 1.4 mmol) in 8 mL SOCl₂ was heated to reflux for 2.5 hours, and the mixture was concentrated to dryness to get a light yellow solid, yield was 98%;

### (c) Synthesis of N-(3-(2-oxopropyl)-1,2,4-thiadiazol-5-yl)-5-(3-(trifluoromethyl)phenyl)thio-phene-3-carboxamide (Compound D-1)

A suspension of compound compound IX-3-1 (408 mg, 1.4 mmol) and potassium thiocyanate (150 mg, 1.5 mmol) in 10 mL acetonitrile was stirred at 60° C. for 30 min, and then 5-methylisoxazol-3-amine (137 mg, 1.4 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 71%; ¹H NMR (500 MHZ, Chloroform-d): δ 8.03 (d, J=1.5 Hz, 1H), 7.92 (d, J=1.5 Hz, 1H), 7.82 (t, J=1.5 Hz, 1H), 7.72-7.68 (m, 1H), 7.68-7.64 (m, 1H), 7.59 (t, J=7.5 Hz, 1H), 3.57 (s, 2H), 2.28 (s, 3H); ESA-MS: m/z=412.0 [M+H]⁺.

### 5-(3-Cyanophenyl)-N-(3-(2-oxopropyl)-1,2,4-thiadiazol-5-yl)thiophene-3-carboxamide (Compound D-2)

Preparation of compound D-2 referred to the synthesis of compound D-1, replacing (3-(trifluoromethyl)phenyl) boronic acid with (3-cyanophenyl) boronic acid to obtain a light yellow solid, yield was 41%; ESA-MS: m/z=369.1 [M+H]⁺.

### 5-(3-Methoxyphenyl)-N-(3-(2-oxopropyl)-1,2,4-thiadiazol-5-yl)thiophene-3-carboxamide (Compound D-3)

Preparation of compound D-3 referred to the synthesis of compound D-1, replacing (3-(trifluoromethyl)phenyl) boronic acid with (3-methoxyphenyl) boronic acid to obtain a light yellow solid, yield was 35%; ESA-MS: m/z=374.1 [M+H]⁺.

### N-(3-(2-oxopropyl)-1,2,4-thiadiazol-5-yl)-5-(3-(trifluoromethoxy)phenyl)thiophene-3-carboxamide (Compound D-4)

Preparation of compound D-4 referred to the synthesis of compound D-1, replacing (3-(trifluoromethyl)phenyl) boronic acid with (3-(trifluoromethoxy)phenyl) boronic acid to obtain a light yellow solid, yield was 32%; ESA-MS: m/z=428.0 [M+H]⁺.

### 5-(3-(Difluoromethoxy)phenyl)-N-(3-(2-oxopropyl)-1,2,4-thiadiazol-5-yl)thiophene-3-carboxamide (Compound D-5)

Preparation of compound D-5 referred to the synthesis of compound D-1, replacing (3-(trifluoromethyl)phenyl) boronic acid with (3-(difluoromethoxy)phenyl) boronic acid to obtain a light yellow solid, yield was 40%; ESA-MS: m/z=410.0 [M+H]⁺.

### Example 34: Synthesis of Compounds D-6˜D-19

The synthesis route is shown in FIG. 40.

### N-(3-(2-(dimethylamino)propyl)-1,2,4-thiadiazol-5-yl)-5-(3-(trifluoromethyl)phenyl)thiophene-3-carboxamide (Compound D-6)

Preparation of compound D-6 referred to the synthesis of compound A-38, using compound D-1 and dimethylamine hydrochloride as the starting material to obtain a light yellow solid, yield was 70%; ESA-MS: m/z=441.1 [M+H]⁺.

### N-(3-(2-(diethylamino)propyl)-1,2,4-thiadiazol-5-yl)-5-(3-(trifluoromethyl)phenyl)thiophene-3-carboxamide (Compound D-7)

Preparation of compound D-7 referred to the synthesis of compound A-38, using compound D-1 and diethylamine hydrochloride as the starting material to obtain a light yellow solid, yield was 67%; ESA-MS: m/z=469.1 [M+H]⁺.

### N-(3-(2-(pyrrolidin-1-yl)propyl)-1,2,4-thiadiazol-5-yl)-5-(3-(trifluoromethyl)phenyl)thiophene-3-carboxamide (Compound D-8)

Preparation of compound D-8 referred to the synthesis of compound A-38, using compound D-1 and pyrrolidine as the starting material to obtain a light yellow solid, yield was 69%; ESA-MS: m/z=467.1 [M+H]⁺.

### N-(3-(2-(piperidin-1-yl)propyl)-1,2,4-thiadiazol-5-yl)-5-(3-(trifluoromethyl)phenyl) thiophene-3-carboxamide (Compound D-9)

Preparation of compound D-9 referred to the synthesis of compound A-38, using compound D-1 and piperidine as the starting material to obtain a light yellow solid, yield was 73%; ESA-MS: m/z=481.1 [M+H]⁺.

### N-(3-(2-morpholinopropyl)-1,2,4-thiadiazol-5-yl)-5-(3-(trifluoromethyl)phenyl)thiophene-3-carboxamide (Compound D-10)

Preparation of compound D-10 referred to the synthesis of compound A-38, using compound D-1 and morpholine as the starting material to obtain a light yellow solid, yield was 74%; ESA-MS: m/z=483.1 [M+H]⁺.

### N-(3-(2-(4-methylpiperazin-1-yl)propyl)-1,2,4-thiadiazol-5-yl)-5-(3-(trifluoromethyl)phenyl)thiophene-3-carboxamide (Compound D-11)

Preparation of compound D-11 referred to the synthesis of compound A-38, using compound D-1 and 1-methylpiperazine as the starting material to obtain a light yellow solid, yield was 68%; ESA-MS: m/z=496.2 [M+H]⁺.

### 5-(3-Cyanophenyl)-N-(3-(2-(pyrrolidin-1-yl)propyl)-1,2,4-thiadiazol-5-yl)thiophene-3-carboxamide (Compound D-12)

Preparation of compound D-12 referred to the synthesis of compound A-38, using compound D-2 and pyrrolidine as the starting material to obtain a light yellow solid, yield was 66%; ESA-MS: m/z=424.1 [M+H]⁺.

### 5-(3-Cyanophenyl)-N-(3-(2-morpholinopropyl)-1,2,4-thiadiazol-5-yl)thiophene-3-carboxamide (Compound D-13)

Preparation of compound D-13 referred to the synthesis of compound A-38, using compound D-2 and morpholine as the starting material to obtain a light yellow solid, yield was 71%; ESA-MS: m/z=440.1 [M+H]⁺.

### 5-(3-Methoxyphenyl)-N-(3-(2-(piperidin-1-yl)propyl)-1,2,4-thiadiazol-5-yl)thiophene-3-carboxamide (Compound D-14)

Preparation of compound D-14 referred to the synthesis of compound A-38, using compound D-3 and piperidine as the starting material to obtain a light yellow solid, yield was 77%; ESA-MS: m/z=443.2 [M+H]⁺.

### 5-(3-Methoxyphenyl)-N-(3-(2-morpholinopropyl)-1,2,4-thiadiazol-5-yl)thiophene-3-carboxamide (Compound D-15)

Preparation of compound D-15 referred to the synthesis of compound A-38, using compound D-3 and morpholine as the starting material to obtain a light yellow solid, yield was 69%; ESA-MS: m/z=445.1 [M+H]⁺.

### N-(3-(2-morpholinopropyl)-1,2,4-thiadiazol-5-yl)-5-(3-(trifluoromethoxy)phenyl)thiophene-3-carboxamide (Compound D-16)

Preparation of compound D-16 referred to the synthesis of compound A-38, using compound D-4 and morpholine as the starting material to obtain a light yellow solid, yield was 63%; ESA-MS: m/z=499.1 [M+H]⁺.

### N-(3-(2-(4-methylpiperazin-1-yl)propyl)-1,2,4-thiadiazol-5-yl)-5-(3-(trifluoromethoxy)phenyl)thiophene-3-carboxamide (Compound D-17)

Preparation of compound D-17 referred to the synthesis of compound A-38, using compound D-4 and 1-methylpiperazine as the starting material to obtain a light yellow solid, yield was 72%; ESA-MS: m/z=512.1 [M+H]⁺.

### 5-(3-(Difluoromethoxy)phenyl)-N-(3-(2-morpholinopropyl)-1,2,4-thiadiazol-5-yl)thiophene-3-carboxamide (Compound D-18)

Preparation of compound D-18 referred to the synthesis of compound A-38, using compound D-5 and morpholine as the starting material to obtain a light yellow solid, yield was 67%; ESA-MS: m/z=481.1 [M+H]⁺.

### 5-(3-(Difluoromethoxy)phenyl)-N-(3-(2-(4-methylpiperazin-1-yl)propyl)-1,2,4-thiadiazol-5-yl)thiophene-3-carboxamide (Compound D-19)

Preparation of compound D-19 referred to the synthesis of compound A-38, using compound D-5 and 1-methylpiperazine as the starting material to obtain a light yellow solid, yield was 68%; ESA-MS: m/z=494.1 [M+H]⁺.

### Example 35: Synthesis of Compounds D-20˜D-24

The synthesis route is shown in FIG. 41.

### N-(3-(2-hydroxypropyl)-1,2,4-thiadiazol-5-yl)-5-(3-(trifluoromethyl)phenyl)thiophene-3-carboxamide (Compound D-20)

Preparation of compound D-20 referred to the synthesis of compound A-60, using compound D-1 as the starting material to obtain a light yellow solid, yield was 83%; ESA-MS: m/z=414.1 [M+H]⁺.

### 5-(3-Cyanophenyl)-N-(3-(2-hydroxypropyl)-1,2,4-thiadiazol-5-yl)thiophene-3-carboxamide (Compound D-21)

Preparation of compound D-21 referred to the synthesis of compound A-60, using compound D-2 as the starting material to obtain a light yellow solid, yield was 85%; ESA-MS: m/z=371.1 [M+H]⁺.

### N-(3-(2-hydroxypropyl)-1,2,4-thiadiazol-5-yl)-5-(3-methoxyphenyl)thiophene-3-carboxamide (Compound D-22)

Preparation of compound D-22 referred to the synthesis of compound A-60, using compound D-3 as the starting material to obtain a light yellow solid, yield was 77%; ESA-MS: m/z=376.1 [M+H]⁺.

### N-(3-(2-hydroxypropyl)-1,2,4-thiadiazol-5-yl)-5-(3-(trifluoromethoxy)phenyl)thiophene-3-carboxamide (Compound D-23)

Preparation of compound D-23 referred to the synthesis of compound A-60, using compound D-4 as the starting material to obtain a light yellow solid, yield was 75%; ESA-MS: m/z=430.0 [M+H]⁺.

### 5-(3-(Difluoromethoxy)phenyl)-N-(3-(2-hydroxypropyl)-1,2,4-thiadiazol-5-yl)thiophene-3-carboxamide (Compound D-24)

Preparation of compound D-24 referred to the synthesis of compound A-60, using compound D-5 as the starting material to obtain a light yellow solid, yield was 82%; ESA-MS: m/z=412.1 [M+H]⁺.

### Example 36: Synthesis of Compounds D-25˜D-29

The synthesis route is shown in FIG. 42.

### N-(3-(3,3,3-trifluoro-2-hydroxy-2-methylpropyl)-1,2,4-thiadiazol-5-yl)-5-(3-(trifluoro-methyl)phenyl)thiophene-3-carboxamide (Compound D-25)

Preparation of compound D-25 referred to the synthesis of compound A-75, using compound D-1 as the starting material to obtain a light yellow solid, yield was 72%; ESA-MS: m/z=482.0 [M+H]⁺.

### 5-(3-Cyanophenyl)-N-(3-(3,3,3-trifluoro-2-hydroxy-2-methylpropyl)-1,2,4-thiadiazol-5-yl)thiophene-3-carboxamide (Compound D-26)

Preparation of compound D-26 referred to the synthesis of compound A-75, using compound D-2 as the starting material to obtain a light yellow solid, yield was 73%; ESA-MS: m/z=439.0 [M+H]⁺.

### 5-(3-Methoxyphenyl)-N-(3-(3,3,3-trifluoro-2-hydroxy-2-methylpropyl)-1,2,4-thiadiazol-5-yl)thiophene-3-carboxamide (Compound D-27)

Preparation of compound D-27 referred to the synthesis of compound A-75, using compound D-3 as the starting material to obtain a light yellow solid, yield was 75%; ESA-MS: m/z=444.1 [M+H]⁺.

### N-(3-(3,3,3-trifluoro-2-hydroxy-2-methylpropyl)-1,2,4-thiadiazol-5-yl)-5-(3-(trifluoro-methoxy)phenyl)thiophene-3-carboxamide (Compound D-28)

Preparation of compound D-28 referred to the synthesis of compound A-75, using compound D-4 as the starting material to obtain a light yellow solid, yield was 68%; ESA-MS: m/z=498.0 [M+H]⁺.

### 5-(3-(Difluoromethoxy)phenyl)-N-(3-(3,3,3-trifluoro-2-hydroxy-2-methylpropyl)-1,2,4-thiadiazol-5-yl)thiophene-3-carboxamide (Compound D-29)

Preparation of compound D-29 referred to the synthesis of compound A-75, using compound D-5 as the starting material to obtain a light yellow solid, yield was 69%; ESA-MS: m/z=480.0 [M+H]⁺.

### Example 37: Synthesis of Compounds D-30˜D-34

The synthesis route is shown in FIG. 43.

### N-(3-(3,3-difluoro-2-methylallyl)-1,2,4-thiadiazol-5-yl)-5-(3-(trifluoromethyl)phenyl)thiophene-3-carboxamide (Compound D-30)

Preparation of compound D-30 referred to the synthesis of compound A-80, using compound D-1 as the starting material to obtain a light yellow solid, yield was 63%; ESA-MS: m/z=446.0 [M+H]⁺.

### 5-(3-Cyanophenyl)-N-(3-(3,3-difluoro-2-methylallyl)-1,2,4-thiadiazol-5-yl)thiophene-3-carboxamide (Compound D-31)

Preparation of compound D-31 referred to the synthesis of compound A-80, using compound D-2 as the starting material to obtain a light yellow solid, yield was 62%; ESA-MS: m/z=403.0 [M+H]⁺.

### N-(3-(3,3-difluoro-2-methylallyl)-1,2,4-thiadiazol-5-yl)-5-(3-methoxyphenyl)thiophene-3-carboxamide (Compound D-32)

Preparation of compound D-32 referred to the synthesis of compound A-80, using compound D-3 as the starting material to obtain a light yellow solid, yield was 65%; ESA-MS: m/z=408.1 [M+H]⁺.

### N-(3-(3,3-difluoro-2-methylallyl)-1,2,4-thiadiazol-5-yl)-5-(3-(trifluoromethoxy)phenyl)thiophene-3-carboxamide (Compound D-33)

Preparation of compound D-33 referred to the synthesis of compound A-80, using compound D-4 as the starting material to obtain a light yellow solid, yield was 67%; ESA-MS: m/z=462.0 [M+H]⁺.

### N-(3-(3,3-difluoro-2-methylallyl)-1,2,4-thiadiazol-5-yl)-5-(3-(difluoromethoxy)phenyl) thiophene-3-carboxamide (Compound D-34)

Preparation of compound D-34 referred to the synthesis of compound A-80, using compound D-5 as the starting material to obtain a light yellow solid, yield was 72%; ESA-MS: m/z=444.0 [M+H]⁺.

### Example 38: Synthesis of Compounds D-35˜D-39

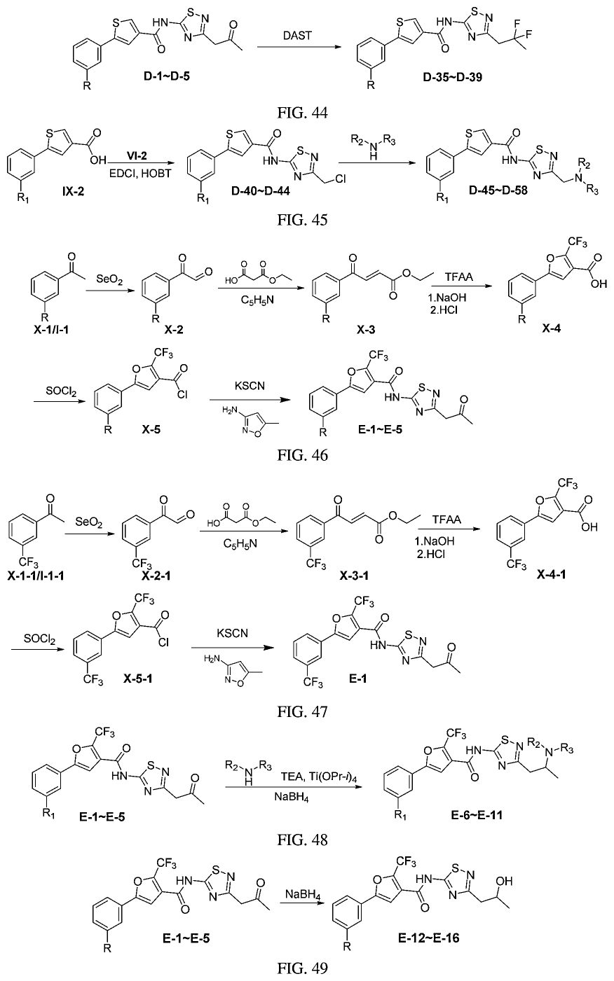

The synthesis route is shown in FIG. 44.

### N-(3-(2,2-difluoropropyl)-1,2,4-thiadiazol-5-yl)-5-(3-(trifluoromethyl)phenyl)thiophene-3-carboxamide

### (Compound D-35)

Preparation of compound D-35 referred to the synthesis of compound A-85, using compound D-1 as the starting material to obtain a light yellow solid, yield was 59%; ESA-MS: m/z=434.0 [M+H]⁺.

### 5-(3-Cyanophenyl)-N-(3-(2,2-difluoropropyl)-1,2,4-thiadiazol-5-yl)thiophene-3-carboxamide (Compound D-36)

Preparation of compound D-36 referred to the synthesis of compound A-85, using compound D-2 as the starting material to obtain a light yellow solid, yield was 58%; ESA-MS: m/z=391.0 [M+H]⁺.

### N-(3-(2,2-difluoropropyl)-1,2,4-thiadiazol-5-yl)-5-(3-methoxyphenyl)thiophene-3-carboxamide (Compound D-37)

Preparation of compound D-37 referred to the synthesis of compound A-85, using compound D-3 as the starting material to obtain a light yellow solid, yield was 60%; ESA-MS: m/z=396.1 [M+H]⁺.

### N-(3-(2,2-difluoropropyl)-1,2,4-thiadiazol-5-yl)-5-(3-(trifluoromethoxy)phenyl)thiophene-3-carboxamide (Compound D-38)

Preparation of compound D-38 referred to the synthesis of compound A-85, using compound D-4 as the starting material to obtain a light yellow solid, yield was 62%; ESA-MS: m/z=450.0 [M+H]⁺.

### 5-(3-(Difluoromethoxy)phenyl)-N-(3-(2,2-difluoropropyl)-1,2,4-thiadiazol-5-yl)thiophene-3-carboxamide (Compound D-39)

Preparation of compound D-39 referred to the synthesis of compound A-85, using compound D-5 as the starting material to obtain a light yellow solid, yield was 57%; ESA-MS: m/z=432.0 [M+H]⁺.

### Example 39: Synthesis of Compounds D-40˜D-58

The synthesis route is shown in FIG. 45.

### N-(3-(chloromethyl)-1,2,4-thiadiazol-5-yl)-5-(3-(trifluoromethyl)phenyl)thiophene-3-carboxamide (Compound D-40)

Preparation of compound D-40 referred to the synthesis of compound A-105, using 5-(3-(trifluoromethyl)phenyl)thiophene-3-carboxylic acid (compound IX-2-1) as the starting material to obtain a light yellow solid, yield was 30%; ESA-MS: m/z=404.0 [M+H]⁺.

### N-(3-(chloromethyl)-1,2,4-thiadiazol-5-yl)-5-(3-cyanophenyl)thiophene-3-carboxamide (Compound D-41)

Preparation of compound D-41 referred to the synthesis of compound A-105, using 5-(3-cyanophenyl)thiophene-3-carboxylic acid (compound IX-2-2) as the starting material to obtain a light yellow solid, yield was 25%; ESA-MS: m/z=361.0 [M+H]⁺.

### N-(3-(chloromethyl)-1,2,4-thiadiazol-5-yl)-5-(3-methoxyphenyl)thiophene-3-carboxamide (Compound D-42)

Preparation of compound D-42 referred to the synthesis of compound A-105, using 5-(3-methoxyphenyl)thiophene-3-carboxylic acid (compound IX-2-3) as the starting material to obtain a light yellow solid, yield was 32%; ESA-MS: m/z=366.0 [M+H]⁺.

### N-(3-(chloromethyl)-1,2,4-thiadiazol-5-yl)-5-(3-(trifluoromethoxy)phenyl)thiophene-3-carboxamide (Compound D-43)

Preparation of compound D-43 referred to the synthesis of compound A-105, using 5-(3-(trifluoromethoxy)phenyl)thiophene-3-carboxylic acid (compound IX-2-4) as the starting material to obtain a light yellow solid, yield was 33%; ESA-MS: m/z=420.0 [M+H]⁺.

### N-(3-(chloromethyl)-1,2,4-thiadiazol-5-yl)-5-(3-(difluoromethoxy)phenyl)thiophene-3-carboxamide (Compound D-44)

Preparation of compound D-44 referred to the synthesis of compound A-105, using 5-(3-(difluoromethoxy)phenyl)thiophene-3-carboxylic acid (compound IX-2-5) as the starting material to obtain a light yellow solid, yield was 29%; ESA-MS: m/z=402.0 [M+H]⁺.

### N-(3-(morpholinomethyl)-1,2,4-thiadiazol-5-yl)-5-(3-(trifluoromethyl)phenyl)thiophene-3-carboxamide (Compound D-45)

Preparation of compound D-45 referred to the synthesis of compound A-106, using compound D-40 and morpholine as the starting material to obtain a light yellow solid, yield was 78%; ESA-MS: m/z=455.1 [M+H]⁺.

### N-(3-((4-methylpiperazin-1-yl)methyl)-1,2,4-thiadiazol-5-yl)-5-(3-(trifluoromethyl)phenyl)thiophene-3-carboxamide (Compound D-46)

Preparation of compound D-46 referred to the synthesis of compound A-106, using compound D-40 and 1-methylpiperazine as the starting material to obtain a light yellow solid, yield was 79%; ESA-MS: m/z=468.1 [M+H]⁺.

### 5-(3-Cyanophenyl)-N-(3-((dimethylamino)methyl)-1,2,4-thiadiazol-5-yl)thiophene-3-carboxamide (Compound D-47)

Preparation of compound D-47 referred to the synthesis of compound A-106, using compound D-41 and dimethylamine hydrochloride as the starting material to obtain a light yellow solid, yield was 66%; ESA-MS: m/z=370.1 [M+H]⁺.

### 5-(3-Cyanophenyl)-N-(3-(morpholinomethyl)-1,2,4-thiadiazol-5-yl)thiophene-3-carboxamide (Compound D-48)

Preparation of compound D-48 referred to the synthesis of compound A-106, using compound D-41 and morpholine as the starting material to obtain a light yellow solid, yield was 74%; ESA-MS: m/z=412.1 [M+H]⁺.

### 5-(3-Cyanophenyl)-N-(3-((4-methylpiperazin-1-yl)methyl)-1,2,4-thiadiazol-5-yl)thiophene-3-carboxamide (Compound D-49)

Preparation of compound D-49 referred to the synthesis of compound A-106, using compound D-41 and 1-methylpiperazine as the starting material to obtain a light yellow solid, yield was 70%; ESA-MS: m/z=425.1 [M+H]⁺.

### N-(3-((diethylamino)methyl)-1,2,4-thiadiazol-5-yl)-5-(3-methoxyphenyl)thiophene-3-carboxamide (Compound D-50)

Preparation of compound D-50 referred to the synthesis of compound A-106, using compound D-42 and dicthylamine hydrochloride as the starting material to obtain a light yellow solid, yield was 68%; ESA-MS: m/z=403.1 [M+H]⁺.

### 5-(3-Methoxyphenyl)-N-(3-(morpholinomethyl)-1,2,4-thiadiazol-5-yl)thiophene-3-carboxamide (Compound D-51)

Preparation of compound D-51 referred to the synthesis of compound A-106, using compound D-42 and morpholine as the starting material to obtain a light yellow solid, yield was 65%; ESA-MS: m/z=417.1 [M+H]⁺.

### 5-(3-Methoxyphenyl)-N-(3-((4-methylpiperazin-1-yl)methyl)-1,2,4-thiadiazol-5-yl)thiophene-3-carboxamide (Compound D-52)

Preparation of compound D-52 referred to the synthesis of compound A-106, using compound D-42 and 1-methylpiperazine as the starting material to obtain a light yellow solid, yield was 72%; ESA-MS: m/z=430.1 [M+H]⁺.

### N-(3-(pyrrolidin-1-ylmethyl)-1,2,4-thiadiazol-5-yl)-5-(3-(trifluoromethoxy)phenyl)thiophene-3-carboxamide (Compound D-53)

Preparation of compound D-53 referred to the synthesis of compound A-106, using compound D-43 and pyrrolidine as the starting material to obtain a light yellow solid, yield was 75%; ESA-MS: m/z=455.1 [M+H]⁺.

### N-(3-(morpholinomethyl)-1,2,4-thiadiazol-5-yl)-5-(3-(trifluoromethoxy)phenyl)thiophene-3-carboxamide (Compound D-54)

Preparation of compound D-54 referred to the synthesis of compound A-106, using compound D-43 and morpholine as the starting material to obtain a light yellow solid, yield was 63%; ESA-MS: m/z=471.1 [M+H]⁺.

### N-(3-((4-methylpiperazin-1-yl)methyl)-1,2,4-thiadiazol-5-yl)-5-(3-(trifluoromethoxy)phenyl)thiophene-3-carboxamide (Compound D-55)

Preparation of compound D-55 referred to the synthesis of compound A-106, using compound D-43 and 1-methylpiperazine as the starting material to obtain a light yellow solid, yield was 71%; ESA-MS: m/z=484.1 [M+H]⁺.

### 5-(3-(Difluoromethoxy)phenyl)-N-(3-(piperidin-1-ylmethyl)-1,2,4-thiadiazol-5-yl)thiophene-3-carboxamide (Compound D-56)

Preparation of compound D-56 referred to the synthesis of compound A-106, using compound D-44 and piperidine as the starting material to obtain a light yellow solid, yield was 69%; ESA-MS: m/z=451.1 [M+H]⁺.

### 5-(3-(Difluoromethoxy)phenyl)-N-(3-(morpholinomethyl)-1,2,4-thiadiazol-5-yl)thiophene-3-carboxamide (Compound D-57)

Preparation of compound D-57 referred to the synthesis of compound A-106, using compound D-44 and morpholine as the starting material to obtain a light yellow solid, yield was 65%; ESA-MS: m/z=453.1 [M+H]⁺.

### 5-(3-(Difluoromethoxy)phenyl)-N-(3-((4-methylpiperazin-1-yl)methyl)-1,2,4-thiadiazol-5-yl)thiophene-3-carboxamide (Compound D-58)

Preparation of compound D-58 referred to the synthesis of compound A-106, using compound D-44 and 1-methylpiperazine as the starting material to obtain a light yellow solid, yield was 74%; ESA-MS: m/z=466.1 [M+H]⁺.

### Example 40: Synthesis of Compounds E-1˜E-5

The synthesis route is shown in FIG. 46.

### N-(3-(2-oxopropyl)-1,2,4-thiadiazol-5-yl)-2-(trifluoromethyl)-5-(3-(trifluoro-methyl)phenyl)furan-3-carboxamide (Compound E-1)

The specific synthesis route is shown in FIG. 47.

### (a) Synthesis of 2-oxo-2-(3-(trifluoromethyl)phenyl) acetaldehyde (Compound X-2-1)

A suspension of selenium dioxide (590 mg, 5.3 mmol) in 1,4-dioxane and water (95:5, 10 mL) was stirred at 60° C. for 3 hours. M-trifluoromethylacetophenone (X-1-1/I-1-1, 1.00 g, 5.3 mmol) was then added and the reaction mixture was heated to reflux for 4 hours. Ethyl acetate was added to the reaction mixture and the organic layer was washed with water and brine, dried over anhydrous Na₂SO₄, and concentrated to give a residue, which was purified by column-chromatograph to give a light brown solid, yield was 65%; ESA-MS: m/z=203.0 [M+H]⁺.

### (b) Synthesis of ethyl (E)-4-oxo-4-(3-(trifluoromethyl)phenyl) but-2-enoate (Compound X-3-1)

To a solution of compound X-2-1 (650 mg, 3.2 mmol) in acetonitrile and water (9:1, 10 mL) was added 3-ethoxy-3-oxopropanoic acid (386 mg, 2.9 mmol) and pyridine (23 mg, 0.29 mmol) and the reaction mixture was stirred at 80° C. for 6 hours. Ethyl acetate was added to the reaction mixture and the organic layer was washed with water and brine, dried over anhydrous Na₂SO₄, and concentrated to give a residue, which was purified by column-chromatograph to give a light yellow solid, yield was 74%; ESA-MS: m/z=273.1 [M+H]⁺.

### (c) Synthesis of 2-(trifluoromethyl)-5-(3-(trifluoromethyl)phenyl)furan-3-carboxylic acid (Compound X-4-1)

To a solution of triphenylphosphine (559 mg, 2.1 mmol) and compound X-3-1 (580 mg, 2.1 mmol) in anhydrous DCM (10 mL) was added trifluoroacetic anhydride (671 mg, 3.2 mmol) dropwise under nitrogen atmosphere and the reaction mixture was stirred at room temperature for 1 hour. After removing the solvent under reduced pressure, a solution of sodium hydroxide (383 mg, 9.6 mmol) in ethanol and water (8:2, 10 mL) was added and stirred at room temperature for 3 hours. The reaction mixture was adjusted with 2 mol/L HCl to acidity, and ethyl acetate was added. The mixture was washed with water and brine, dried over anhydrous Na₂SO₄, and concentrated to give a light solid, yield was 64%; ESA-MS: m/z=325.0 [M+H]⁺.

### (d) Synthesis of 2-(trifluoromethyl)-5-(3-(trifluoromethyl)phenyl)furan-3-carbonyl chloride (Compound X-5-1)

A solution of compound X-4-1 (440 mg, 1.4 mmol) in 10 mL SOCl₂ was heated to reflux for 2.5 hours, and the mixture was concentrated to dryness to get a light yellow solid, yield was 94%;

### (e) Synthesis of N-(3-(2-oxopropyl)-1,2,4-thiadiazol-5-yl)-2-(trifluoromethyl)-5-(3-(trifluoro-methyl)phenyl)furan-3-carboxamide (Compound E-1)

A suspension of compound X-5-1 (437 mg, 1.3 mmol) and potassium thiocyanate (136 mg, 1.4 mmol) in 10 mL acetonitrile was stirred at 60° C. for 30 min, and then 5-methylisoxazol-3-amine (125 mg, 1.3 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 85%; ¹H NMR (500 MHZ, Chloroform-d): δ 8.09 (t, J=1.5 Hz, 1H), 7.79-7.75 (m, 1H), 7.72-7.68 (m, 1H), 7.58 (t, J=7.5 Hz, 1H), 7.42 (s, 1H), 3.58 (s, 2H), 2.32 (s, 3H); ESA-MS: m/z=464.0 [M+H]⁺.

### 5-(3-Cyanophenyl)-N-(3-(2-oxopropyl)-1,2,4-thiadiazol-5-yl)-2-(trifluoromethyl)furan-3-carboxamide (Compound E-2)

Preparation of compound E-2 referred to the synthesis of compound E-1, replacing 1-(3-(trifluoromethyl)phenyl)ethan-1-one with 3-acetylbenzonitrile to obtain a light yellow solid, yield was 21%; ESA-MS: m/z=421.1 [M+H]⁺.

### 5-(3-Methoxyphenyl)-N-(3-(2-oxopropyl)-1,2,4-thiadiazol-5-yl)-2-(trifluoromethyl)furan-3-carboxamide (Compound E-3)

Preparation of compound E-3 referred to the synthesis of compound E-1, replacing 1-(3-(trifluoromethyl)phenyl)ethan-1-one with 1-(3-methoxyphenyl) ethan-1-one to obtain a light yellow solid, yield was 24%; ESA-MS: m/z=426.1 [M+H]⁺.

### N-(3-(2-oxopropyl)-1,2,4-thiadiazol-5-yl)-5-(3-(trifluoromethoxy)phenyl)-2-(trifluoro-methyl)furan-3-carboxamide (Compound E-4)

Preparation of compound E-4 referred to the synthesis of compound E-1, replacing 1-(3-(trifluoromethyl)phenyl)ethan-1-one with 1-(3-(trifluoromethoxy)phenyl) ethan-1-one to obtain a light yellow solid, yield was 25%; ESA-MS: m/z=480.0 [M+H]⁺.

### 5-(3-(Difluoromethoxy)phenyl)-N-(3-(2-oxopropyl)-1,2,4-thiadiazol-5-yl)-2-(trifluoro-methyl)furan-3-carboxamide (Compound E-5)

Preparation of compound E-5 referred to the synthesis of compound E-1, replacing 1-(3-(trifluoromethyl)phenyl)ethan-1-one with 1-(3-(difluoromethoxy)phenyl) ethan-1-one to obtain a light yellow solid, yield was 30%; ESA-MS: m/z=462.0 [M+H]⁺.

### Example 41: Synthesis of Compounds E-6˜E-11

The synthesis route is shown in FIG. 48.

### N-(3-(2-(pyrrolidin-1-yl)propyl)-1,2,4-thiadiazol-5-yl)-2-(trifluoromethyl)-5-(3-(trifluoromethyl)phenyl) furan-3-carboxamide (Compound E-6)

Preparation of compound E-6 referred to the synthesis of compound A-38, using compound E-1 and pyrrolidine as the starting material to obtain a light yellow solid, yield was 63%; ESA-MS: m/z=519.1 [M+H]⁺.

### N-(3-(2-(4-methylpiperazin-1-yl)propyl)-1,2,4-thiadiazol-5-yl)-2-(trifluoromethyl)-5-(3-(trifluoromethyl)phenyl)furan-3-carboxamide (Compound E-7)

Preparation of compound E-7 referred to the synthesis of compound A-38, using compound E-1 and 1-methylpiperazine as the starting material to obtain a light yellow solid, yield was 65%; ESA-MS: m/z=548.1 [M+H]⁺.

### 5-(3-Cyanophenyl)-N-(3-(2-morpholinopropyl)-1,2,4-thiadiazol-5-yl)-2-(trifluoromethyl)furan-3-carboxamide (Compound E-8)

Preparation of compound E-8 referred to the synthesis of compound A-38, using compound E-2 and morpholine as the starting material to obtain a light yellow solid, yield was 70%; ESA-MS: m/z=492.1 [M+H]⁺.

### 5-(3-Methoxyphenyl)-N-(3-(2-(piperidin-1-yl)propyl)-1,2,4-thiadiazol-5-yl)-2-(trifluoro-methyl)furan-3-carboxamide (Compound E-9)

Preparation of compound E-9 referred to the synthesis of compound A-38, using compound E-3 and piperidine as the starting material to obtain a light yellow solid, yield was 72%; ESA-MS: m/z=495.2 [M+H]⁺.

### N-(3-(2-morpholinopropyl)-1,2,4-thiadiazol-5-yl)-5-(3-(trifluoromethoxy)phenyl)-2-(trifluoromethyl)furan-3-carboxamide (Compound E-10)

Preparation of compound E-10 referred to the synthesis of compound A-38, using compound E-4 and morpholine as the starting material to obtain a light yellow solid, yield was 68%; ESA-MS: m/z=551.1 [M+H]⁺.

### 5-(3-(Difluoromethoxy)phenyl)-N-(3-(2-(4-methylpiperazin-1-yl)propyl)-1,2,4-thiadiazol-5-yl)-2-(trifluo romethyl)furan-3-carboxamide (Compound E-11)

Preparation of compound E-11 referred to the synthesis of compound A-38, using compound E-5 and 1-methylpiperazine as the starting material to obtain a light yellow solid, yield was 66%; ESA-MS: m/z=546.2 [M+H]⁺.

### Example 42: Synthesis of Compounds E-12˜E-16

The synthesis route is shown in FIG. 49.

### N-(3-(2-hydroxypropyl)-1,2,4-thiadiazol-5-yl)-2-(trifluoromethyl)-5-(3-(trifluoromethyl)phenyl)furan-3-carboxamide (Compound E-12)

Preparation of compound E-12 referred to the synthesis of compound A-60, using compound E-1 as the starting material to obtain a light yellow solid, yield was 82%; ESA-MS: m/z=466.1 [M+H]⁺.

### 5-(3-Cyanophenyl)-N-(3-(2-hydroxypropyl)-1,2,4-thiadiazol-5-yl)-2-(trifluoromethyl)furan-3-carboxamide (Compound E-13)

Preparation of compound E-13 referred to the synthesis of compound A-60, using compound E-2 as the starting material to obtain a light yellow solid, yield was 80%; ESA-MS: m/z=423.1 [M+H]⁺.

### N-(3-(2-hydroxypropyl)-1,2,4-thiadiazol-5-yl)-5-(3-methoxyphenyl)-2-(trifluoromethyl) furan-3-carboxamide (Compound E-14)

Preparation of compound E-14 referred to the synthesis of compound A-60, using compound E-3 as the starting material to obtain a light yellow solid, yield was 75%; ESA-MS: m/z=428.1 [M+H]⁺.

### N-(3-(2-hydroxypropyl)-1,2,4-thiadiazol-5-yl)-5-(3-(trifluoromethoxy)phenyl)-2-(trifluoro-methyl)furan-3-carboxamide (Compound E-15)

Preparation of compound E-15 referred to the synthesis of compound A-60, using compound E-4 as the starting material to obtain a light yellow solid, yield was 78%; ESA-MS: m/z=482.1 [M+H]⁺.

### 5-(3-(Difluoromethoxy)phenyl)-N-(3-(2-hydroxypropyl)-1,2,4-thiadiazol-5-yl)-2-(trifluoro-methyl)furan-3-carboxamide (Compound E-16)

Preparation of compound E-16 referred to the synthesis of compound A-60, using compound E-5 as the starting material to obtain a light yellow solid, yield was 81%; ESA-MS: m/z=464.1 [M+H]⁺.

### Example 43: Synthesis of Compounds E-17˜E-21

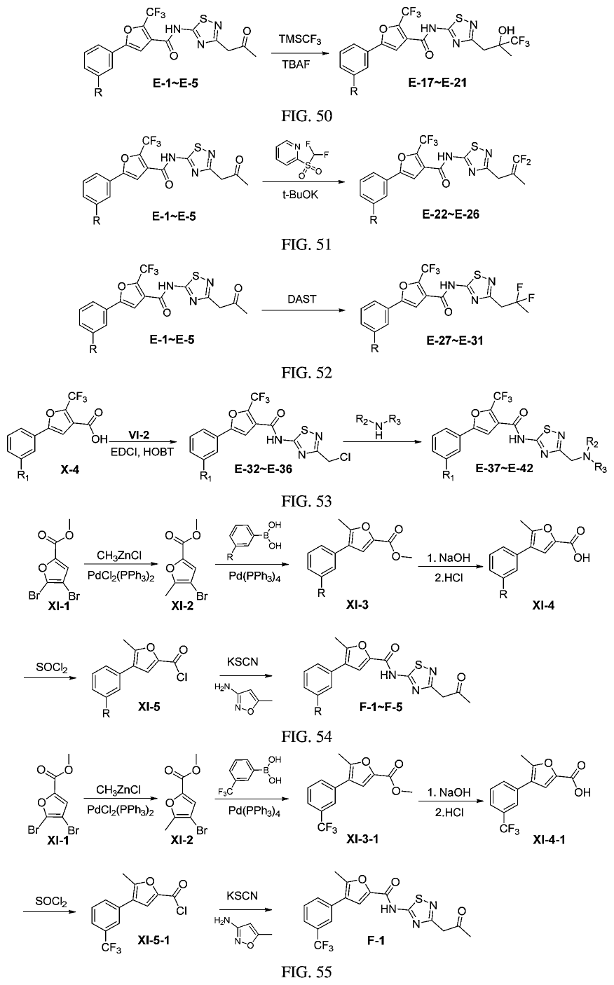

The synthesis route is shown in FIG. 50.

### N-(3-(3,3,3-trifluoro-2-hydroxy-2-methylpropyl)-1,2,4-thiadiazol-5-yl)-2-(trifluoromethyl)-5-(3-(trifluoromethyl)phenyl)furan-3-carboxamide (Compound E-17)

Preparation of compound E-17 referred to the synthesis of compound A-75, using compound E-1 as the starting material to obtain a light yellow solid, yield was 73%; ESA-MS: m/z=534.0 [M+H]⁺.

### 5-(3-Cyanophenyl)-N-(3-(3,3,3-trifluoro-2-hydroxy-2-methylpropyl)-1,2,4-thiadiazol-5-yl)-2-(trifluoromethyl)furan-3-carboxamide (Compound E-18)

Preparation of compound E-18 referred to the synthesis of compound A-75, using compound E-2 as the starting material to obtain a light yellow solid, yield was 74%; ESA-MS: m/z=491.1 [M+H]⁺.

### 5-(3-Methoxyphenyl)-N-(3-(3,3,3-trifluoro-2-hydroxy-2-methylpropyl)-1,2,4-thiadiazol-5-yl)-2-(trifluoro methyl)furan-3-carboxamide (Compound E-19)

Preparation of compound E-19 referred to the synthesis of compound A-75, using compound E-3 as the starting material to obtain a light yellow solid, yield was 75%; ESA-MS: m/z=496.1 [M+H]⁺.

### N-(3-(3,3,3-trifluoro-2-hydroxy-2-methylpropyl)-1,2,4-thiadiazol-5-yl)-5-(3-(trifluoro-methoxy)phenyl)-2-(trifluoromethyl)furan-3-carboxamide (Compound E-20)

Preparation of compound E-20 referred to the synthesis of compound A-75, using compound E-4 as the starting material to obtain a light yellow solid, yield was 62%; ESA-MS: m/z=550.1 [M+H]⁺.

### 5-(3-(Difluoromethoxy)phenyl)-N-(3-(3,3,3-trifluoro-2-hydroxy-2-methylpropyl)-1,2,4-thiadiazol-5-yl)-2-(trifluoromethyl)furan-3-carboxamide (Compound E-21)

Preparation of compound E-21 referred to the synthesis of compound A-75, using compound E-5 as the starting material to obtain a light yellow solid, yield was 69%; ESA-MS: m/z=532.0 [M+H]⁺.

### Example 44: Synthesis of Compounds E-22˜E-26

The synthesis route is shown in FIG. 51.

### N-(3-(3,3-difluoro-2-methylallyl)-1,2,4-thiadiazol-5-yl)-2-(trifluoromethyl)-5-(3-(trifluoromethyl)phenyl) furan-3-carboxamide (Compound E-22)

Preparation of compound E-22 referred to the synthesis of compound A-80, using compound E-1 as the starting material to obtain a light yellow solid, yield was 60%; ESA-MS: m/z=498.0 [M+H]⁺.

### 5-(3-Cyanophenyl)-N-(3-(3,3-difluoro-2-methylallyl)-1,2,4-thiadiazol-5-yl)-2-(trifluoro-methyl)furan-3-carboxamide (Compound E-23)

Preparation of compound E-23 referred to the synthesis of compound A-80, using compound E-2 as the starting material to obtain a light yellow solid, yield was 61%; ESA-MS: m/z=455.1 [M+H]⁺.

### N-(3-(3,3-difluoro-2-methylallyl)-1,2,4-thiadiazol-5-yl)-5-(3-methoxyphenyl)-2-(trifluoro-methyl)furan-3-carboxamide (Compound E-24)

Preparation of compound E-24 referred to the synthesis of compound A-80, using compound E-3 as the starting material to obtain a light yellow solid, yield was 65%; ESA-MS: m/z=460.1 [M+H]⁺.

### N-(3-(3,3-difluoro-2-methylallyl)-1,2,4-thiadiazol-5-yl)-5-(3-(trifluoromethoxy)phenyl)-2-(trifluoromethylfuran-3-carboxamide (Compound E-25)

Preparation of compound E-25 referred to the synthesis of compound A-80, using compound E-4 as the starting material to obtain a light yellow solid, yield was 57%; ESA-MS: m/z=514.0 [M+H]⁺.

### N-(3-(3,3-difluoro-2-methylallyl)-1,2,4-thiadiazol-5-yl)-5-(3-(difluoromethoxy)phenyl)-2-(trifluoromethyl)furan-3-carboxamide (Compound E-26)

Preparation of compound E-26 referred to the synthesis of compound A-80, using compound E-5 as the starting material to obtain a light yellow solid, yield was 72%; ESA-MS: m/z=496.0 [M+H]⁺.

### Example 45: Synthesis of Compounds E-27˜E-31

The synthesis route is shown in FIG. 52.

### N-(3-(2,2-difluoropropyl)-1,2,4-thiadiazol-5-yl)-2-(trifluoromethyl)-5-(3-(trifluoromethyl)phenyl) fura n-3-carboxamide (Compound E-27)

Preparation of compound E-27 referred to the synthesis of compound A-85, using compound E-1 as the starting material to obtain a light yellow solid, yield was 59%; ESA-MS: m/z=486.0 [M+H]⁺.

### 5-(3-Cyanophenyl)-N-(3-(2,2-difluoropropyl)-1,2,4-thiadiazol-5-yl)-2-(trifluoromethyl) furan-3-carboxamide (Compound E-28)

Preparation of compound E-28 referred to the synthesis of compound A-85, using compound E-2 as the starting material to obtain a light yellow solid, yield was 50%; ESA-MS: m/z=443.1 [M+H]⁺.

### N-(3-(2,2-difluoropropyl)-1,2,4-thiadiazol-5-yl)-5-(3-methoxyphenyl)-2-(trifluoromethyl) furan-3-carboxamide (Compound E-29)

Preparation of compound E-29 referred to the synthesis of compound A-85, using compound E-3 as the starting material to obtain a light yellow solid, yield was 60%; ESA-MS: m/z=448.1 [M+H]⁺.

### N-(3-(2,2-difluoropropyl)-1,2,4-thiadiazol-5-yl)-5-(3-(trifluoromethoxy)phenyl)-2-(trifluoromethyl)furan-3-carboxamide (Compound E-30)

Preparation of compound E-30 referred to the synthesis of compound A-85, using compound E-4 as the starting material to obtain a light yellow solid, yield was 61%; ESA-MS: m/z=502.0 [M+H]⁺.

### 5-(3-(Difluoromethoxy)phenyl)-N-(3-(2,2-difluoropropyl)-1,2,4-thiadiazol-5-yl)-2-(trifluoromethyl)furan-3-carboxamide (Compound E-31)

Preparation of compound E-31 referred to the synthesis of compound A-85, using compound E-5 as the starting material to obtain a light yellow solid, yield was 53%; ESA-MS: m/z=484.0 [M+H]⁺.

### Example 46: Synthesis of Compounds E-32˜E-42

The synthesis route is shown in FIG. 53.

### N-(3-(chloromethyl)-1,2,4-thiadiazol-5-yl)-2-(trifluoromethyl)-5-(3-(trifluoromethyl)phenyl)furan-3-carboxamide (Compound E-32)

Preparation of compound E-32 referred to the synthesis of compound A-105, using 2-(trifluoromethyl)-5-(3-(trifluoromethyl)phenyl)furan-3-carboxylic acid (compound X-4-1) as the starting material to obtain a light yellow solid, yield was 33%; ESA-MS: m/z=456.0 [M+H]⁺.

### N-(3-(chloromethyl)-1,2,4-thiadiazol-5-yl)-5-(3-cyanophenyl)-2-(trifluoromethyl)furan-3-carboxamide (Compound E-33)

Preparation of compound E-33 referred to the synthesis of compound A-105, using 5-(3-cyanophenyl)-2-(trifluoromethyl)furan-3-carboxylic acid (compound X-4-2) as the starting material to obtain a light yellow solid, yield was 27%; ESA-MS: m/z=413.0 [M+H]⁺.

### N-(3-(chloromethyl)-1,2,4-thiadiazol-5-yl)-5-(3-methoxyphenyl)-2-(trifluoromethyl)furan-3-carboxamide (Compound E-34)

Preparation of compound referred to the synthesis of compound A-105, using 5-(3-methoxyphenyl)-2-(trifluoromethyl)furan-3-carboxylic acid (compound X-4-3) as the starting material to obtain a light yellow solid, yield was 31%; ESA-MS: m/z=418.0 [M+H]⁺.

### N-(3-(chloromethyl)-1,2,4-thiadiazol-5-yl)-5-(3-(trifluoromethoxy)phenyl)-2-(trifluoro-methyl)furan-3-carboxamide (Compound E-35)

Preparation of compound E-35 referred to the synthesis of compound A-105, using 5-(3-(trifluoromethoxy)phenyl)-2-(trifluoromethyl)furan-3-carboxylic acid (compound X-4-4) as the starting material to obtain a light yellow solid, yield was 35%; ESA-MS: m/z=472.0 [M+H]⁺.

### N-(3-(chloromethyl)-1,2,4-thiadiazol-5-yl)-5-(3-(difluoromethoxy)phenyl)-2-(trifluoro-methyl)furan-3-carboxamide (Compound E-36)

Preparation of compound referred to the synthesis of compound A-105, using 5-(3-(difluoromethoxy)phenyl)-2-(trifluoromethyl)furan-3-carboxylic acid (compound X-4-5) as the starting material to obtain a light yellow solid, yield was 28%; ESA-MS: m/z=454.0 [M+H]⁺.

### N-(3-((4-methylpiperazin-1-yl)methyl)-1,2,4-thiadiazol-5-yl)-2-(trifluoromethyl)-5-(3-(trifluoromethyl)phenyl)furan-3-carboxamide (Compound E-37)

Preparation of compound E-37 referred to the synthesis of compound A-106, using compound E-32 and 1-methylpiperazine as the starting material to obtain a light yellow solid, yield was 74%; ESA-MS: m/z=520.1 [M+H]⁺.

### 5-(3-Cyanophenyl)-N-(3-((4-methylpiperazin-1-yl)methyl)-1,2,4-thiadiazol-5-yl)-2-(trifluoromethyl)furan-3-carboxamide (Compound E-38)

Preparation of compound E-38 referred to the synthesis of compound A-106, using compound E-33 and 1-methylpiperazine as the starting material to obtain a light yellow solid, yield was 69%; ESA-MS: m/z=477.1 [M+H]⁺.

### 5-(3-Methoxyphenyl)-N-(3-(morpholinomethyl)-1,2,4-thiadiazol-5-yl)-2-(trifluoromethyl)furan-3-carboxamide (Compound E-39)

Preparation of compound E-39 referred to the synthesis of compound A-106, using compound E-34 and morpholine as the starting material to obtain a light yellow solid, yield was 65%; ESA-MS: m/z=469.1 [M+H]⁺.

### N-(3-(pyrrolidin-1-ylmethyl)-1,2,4-thiadiazol-5-yl)-5-(3-(trifluoromethoxy)phenyl)-2-(trifluoromethyl)furan-3-carboxamide (Compound E-40)

Preparation of compound E-40 referred to the synthesis of compound A-106, using compound E-35 and pyrrolidine as the starting material to obtain a light yellow solid, yield was 76%; ESA-MS: m/z=507.1 [M+H]⁺.

### 5-(3-(Difluoromethoxy)phenyl)-N-(3-(piperidin-1-ylmethyl)-1,2,4-thiadiazol-5-yl)-2-(trifluoromethyl)furan-3-carboxamide (Compound E-41)

Preparation of compound E-41 referred to the synthesis of compound A-106, using compound E-36 and piperidine as the starting material to obtain a light yellow solid, yield was 63%; ESA-MS: m/z=503.1 [M+H]⁺.

### 5-(3-(Difluoromethoxy)phenyl)-N-(3-(morpholinomethyl)-1,2,4-thiadiazol-5-yl)-2-(trifluoromethyl)furan-3-carboxamide (Compound E-42)

Preparation of compound E-42 referred to the synthesis of compound A-106, using compound E-36 and morpholine as the starting material to obtain a light yellow solid, yield was 68%; ESA-MS: m/z=505.1 [M+H]⁺.

### Example 47: Synthesis of Compounds F-1˜F-5

The synthesis route is shown in FIG. 54.

### 5-Methyl-N-(3-(2-oxopropyl)-1,2,4-thiadiazol-5-yl)-4-(3-(trifluoromethyl)phenyl)furan-2-carboxamide (Compound F-1)

The specific synthesis route is shown in FIG. 55.

### (a) Synthesis of methyl 4-bromo-5-methylfuran-2-carboxylate (Compound XI-2)

To a solution of methyl 4,5-dibromofuran-2-carboxylate (XI-1, 1.00 g, 3.5 mmol) in THF (12 mL) was added bis(triphenylphosphine)palladium(II) chloride (250 mg, 0.35 mmol) and methylzinc chloride (2 mol/L solution in THF, 2.10 mL, 4.2 mmol) and stirred at room temperature for 5 hours. Saturated ammonium chloride solution was then added to quench the reaction. Ethyl acetate was added to the reaction mixture and the organic layer was washed with water and brine, dried over anhydrous Na₂SO₄, and concentrated to give a residue, which was purified by column-chromatograph to give a light brown solid, yield was 85%; ESA-MS: m/z=219.0 [M+H]⁺.

### (b) Synthesis of methyl 5-methyl-4-(3-(trifluoromethyl)phenyl)furan-2-carboxylate (Compound XI-3-1)

To a solution of compound XI-2 (200 mg, 0.91 mmol) in THF (8 mL) was added 3-(trifluoromethyl)phenylboronic acid (208 mg, 1.1 mmol), cesium carbonate (120 mg, 0.73 mmol), [1,1′-bis(diphenylphosphino)ferrocene]dichloropalladium (II) (40 mg, 0.05 mmol) under nitrogen atmosphere, and the reaction mixture was heated to reflux overnight. Ethyl acetate was added to the reaction mixture and the organic layer was washed with water and brine, dried over anhydrous Na₂SO₄, and concentrated to give a residue, which was purified by column-chromatograph to give a light yellow solid, yield was 64%; ESA-MS: m/z=285.1 [M+H]⁺.

### (c) Synthesis of 5-methyl-4-(3-(trifluoromethyl)phenyl)furan-2-carboxylic acid (Compound XI-4-1)

To a solution of compound XI-3-1 (166 mg, 0.58 mmol) in EtOH/H₂O (3:1, 12 mL) was added sodium hydroxide (140 mg, 3.5 mmol) and stirred overnight at room temperature. The reaction mixture was adjusted with 2 mol/L HCl to acidity, and ethyl acetate was added. The mixture was washed with water and brine, dried over anhydrous Na₂SO₄, and concentrated to give a light yellow solid, yield was 91%; ESA-MS: m/z=271.0 [M+H]⁺.

### (d) Synthesis of 5-methyl-4-(3-(trifluoromethyl)phenyl)furan-2-carbonyl chloride (Compound XI-5-1)

A solution of compound XI-4-1 (143 mg, 0.53 mmol) in 8 mL SOCl₂ was heated to reflux for 2.5 hours, and the mixture was concentrated to dryness to get a light yellow solid, yield was 94%.

### (e) Synthesis of 5-methyl-N-(3-(2-oxopropyl)-1,2,4-thiadiazol-5-yl)-4-(3-(trifluoromethyl)phenyl)furan-2-carboxamide (Compound F-1)

A suspension of compound XI-5-1 (144 mg, 0.50 mmol) and potassium thiocyanate (53 mg, 0.55 mmol) in 8 mL acetonitrile was stirred at 60° C. for 30 min, and then 5-methylisoxazol-3-amine (49 mg, 0.50 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 83%.

### 4-(3-Cyanophenyl)-5-methyl-N-(3-(2-oxopropyl)-1,2,4-thiadiazol-5-yl)furan-2-carboxamide (Compound F-2)

Preparation of compound F-2 referred to the synthesis of compound F-1, replacing (3-(trifluoromethyl)phenyl) boronic acid with (3-cyanophenyl) boronic acid to obtain a light yellow solid, yield was 32%; ESA-MS: m/z=367.1 [M+H]⁺.

### 4-(3-Methoxyphenyl)-5-methyl-N-(3-(2-oxopropyl)-1,2,4-thiadiazol-5-yl)furan-2-carboxamide (Compound F-3)

Preparation of compound F-3 referred to the synthesis of compound F-1, replacing (3-(trifluoromethyl)phenyl) boronic acid with (3-methoxyphenyl) boronic acid to obtain a light yellow solid, yield was 30%; ESA-MS: m/z=372.1 [M+H]⁺.

### 5-Methyl-N-(3-(2-oxopropyl)-1,2,4-thiadiazol-5-yl)-4-(3-(trifluoromethoxy)phenyl)furan-2-carboxamide (Compound F-4)

Preparation of compound F-4 referred to the synthesis of compound F-1, replacing (3-(trifluoromethyl)phenyl) boronic acid with (3-(trifluoromethoxy)phenyl) boronic acid to obtain a light yellow solid, yield was 29%; ESA-MS: m/z=426.1 [M+H]⁺.

### 4-(3-(Difluoromethoxy)phenyl)-5-methyl-N-(3-(2-oxopropyl)-1,2,4-thiadiazol-5-yl)furan-2-carboxamide (Compound F-5)

Preparation of compound F-5 referred to the synthesis of compound F-1, replacing (3-(trifluoromethyl)phenyl) boronic acid with (3-(difluoromethoxy)phenyl) boronic acid to obtain a light yellow solid, yield was 31%; ESA-MS: m/z=408.1 [M+H]⁺.

### Example 48: Synthesis of Compounds F-6˜F-10

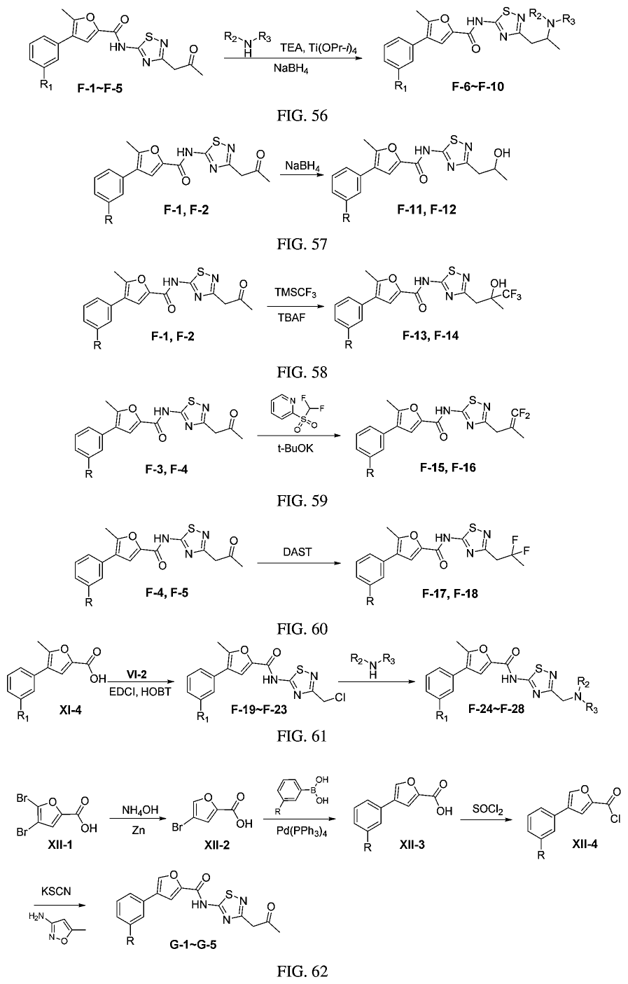

The synthesis route is shown in FIG. 56.

### 5-Methyl-N-(3-(2-(pyrrolidin-1-yl)propyl)-1,2,4-thiadiazol-5-yl)-4-(3-(trifluoromethyl phenyl)furan-2-carboxamide (Compound F-6)

Preparation of compound F-6 referred to the synthesis of compound A-38, using compound F-1 and pyrrolidine as the starting material to obtain a light yellow solid, yield was 63%; ESA-MS: m/z=465.1 [M+H]⁺.

### 4-(3-Cyanophenyl)-5-methyl-N-(3-(2-(piperidin-1-yl)propyl)-1,2,4-thiadiazol-5-yl)furan-2-carboxamide (Compound F-7)

Preparation of compound F-7 referred to the synthesis of compound A-38, using compound F-2 and piperidine as the starting material to obtain a light yellow solid, yield was 73%; ESA-MS: m/z=436.2 [M+H]⁺.

### 4-(3-Methoxyphenyl)-5-methyl-N-(3-(2-morpholinopropyl)-1,2,4-thiadiazol-5-yl)furan-2-carboxamide (Compound F-8)

Preparation of compound F-8 referred to the synthesis of compound A-38, using compound F-3 and morpholine as the starting material to obtain a light yellow solid, yield was 68%; ESA-MS: m/z=443.2 [M+H]⁺.

### 5-Methyl-N-(3-(2-morpholinopropyl)-1,2,4-thiadiazol-5-yl)-4-(3-(trifluoromethoxy)phenyl)furan-2-carboxamide (Compound F-9)

Preparation of compound F-9 referred to the synthesis of compound A-38, using compound F-4 and morpholine as the starting material to obtain a light yellow solid, yield was 66%; ESA-MS: m/z=497.1 [M+H]⁺.

### 4-(3-(Difluoromethoxy)phenyl)-5-methyl-N-(3-(2-(4-methylpiperazin-1-yl)propyl)-1,2,4-thiadiazol-5-yl)furan-2-carboxamide (Compound F-10)

Preparation of compound F-10 referred to the synthesis of compound A-38, using compound F-5 and 1-methylpiperazine as the starting material to obtain a light yellow solid, yield was 69%; ESA-MS: m/z=492.2 [M+H]⁺.

### Example 49: Synthesis of Compound F-11˜F-12

The synthesis route is shown in FIG. 57.

### N-(3-(2-hydroxypropyl)-1,2,4-thiadiazol-5-yl)-5-methyl-4-(3-(trifluoromethyl)phenyl) furan-2-carboxamide (Compound F-11)

Preparation of compound F-11 referred to the synthesis of compound A-60, using compound F-1 as the starting material to obtain a light yellow solid, yield was 85%; ESA-MS: m/z=412.1 [M+H]⁺.

### 4-(3-Cyanophenyl)-N-(3-(2-hydroxypropyl)-1,2,4-thiadiazol-5-yl)-5-methylfuran-2-carboxamide (Compound F-12)

Preparation of compound F-12 referred to the synthesis of compound A-60, using compound F-2 as the starting material to obtain a light yellow solid, yield was 80%; ESA-MS: m/z=369.1 [M+H]⁺.

### Example 50: Synthesis of Compounds F-13˜F-14

The synthesis route is shown in FIG. 58.

### 5-Methyl-N-(3-(3,3,3-trifluoro-2-hydroxy-2-methylpropyl)-1,2,4-thiadiazol-5-yl)-4-(3-(tri-fluoromethyl)phenyl)furan-2-carboxamide (Compound F-13)

Preparation of compound F-13 referred to the synthesis of compound A-75, using compound F-1 as the starting material to obtain a light yellow solid, yield was 72%; ESA-MS: m/z=480.1 [M+H]⁺.

### 4-(3-Cyanophenyl)-5-methyl-N-(3-(3,3,3-trifluoro-2-hydroxy-2-methylpropyl)-1,2,4-thiadiazol-5-yl)furan-2-carboxamide (Compound F-14)

Preparation of compound F-14 referred to the synthesis of compound A-75, using compound F-2 as the starting material to obtain a light yellow solid, yield was 69%; ESA-MS: m/z=437.1 [M+H]⁺.

### Example 51: Synthesis of Compounds F-15˜F-16

The synthesis route is shown in FIG. 59.

### N-(3-(3,3-difluoro-2-methylallyl)-1,2,4-thiadiazol-5-yl)-4-(3-methoxyphenyl)-5-methyl-furan-2-carboxamide (Compound F-15)

Preparation of compound F-15 referred to the synthesis of compound A-80, using compound F-3 as the starting material to obtain a light yellow solid, yield was 69%; ESA-MS: m/z=406.1 [M+H]⁺.

### N-(3-(3,3-difluoro-2-methylallyl)-1,2,4-thiadiazol-5-yl)-5-methyl-4-(3-(trifluoromethoxy)phenyl)furan-2-carboxamide (Compound F-16)

Preparation of compound F-16 referred to the synthesis of compound A-80, using compound F-4 as the starting material to obtain a light yellow solid, yield was 65%; ESA-MS: m/z=460.1 [M+H]⁺.

### Example 52: Synthesis of Compounds F-17˜F-18

The synthesis route is shown in FIG. 60.

### N-(3-(2,2-difluoropropyl)-1,2,4-thiadiazol-5-yl)-5-methyl-4-(3-(trifluoromethoxy)phenyl)furan-2-carboxamide (Compound F-17)

Preparation of compound F-17 referred to the synthesis of compound A-85, using compound F-4 as the starting material to obtain a light yellow solid, yield was 65%; ESA-MS: m/z=448.1 [M+H]⁺. 4-(3-(Difluoromethoxy)phenyl)-N-(3-(2,2-difluoropropyl)-1,2,4-thiadiazol-5-yl)-5-methyl-furan-2-carboxamide (compound F-18)

Preparation of compound F-18 referred to the synthesis of compound A-85, using compound F-5 as the starting material to obtain a light yellow solid, yield was 58%; ESA-MS: m/z=430.1 [M+H]⁺.

### Example 53: Synthesis of Compounds F-19˜F-28

The synthesis route is shown in FIG. 61.

### N-(3-(chloromethyl)-1,2,4-thiadiazol-5-yl)-5-methyl-4-(3-(trifluoromethyl)phenyl)furan-2-carboxamide (Compound F-19)

Preparation of compound F-19 referred to the synthesis of compound A-105, using 5-methyl-4-(3-(trifluoromethyl)phenyl)furan-2-carboxylic acid (compound XI-4-1) as the starting material to obtain a light yellow solid, yield was 33%; ESA-MS: m/z=402.0 [M+H]⁺.

### N-(3-(chloromethyl)-1,2,4-thiadiazol-5-yl)-4-(3-cyanophenyl)-5-methylfuran-2-carboxamide (Compound F-20)

Preparation of compound F-20 referred to the synthesis of compound A-105, using 4-(3-cyanophenyl)-5-methylfuran-2-carboxylic acid (compound XI-4-2) as the starting material to obtain a light yellow solid, yield was 28%; ESA-MS: m/z=359.0 [M+H]⁺.

### N-(3-(chloromethyl)-1,2,4-thiadiazol-5-yl)-4-(3-methoxyphenyl)-5-methylfuran-2-carboxamide (Compound F-21)

Preparation of compound F-21 referred to the synthesis of compound A-105, using 4-(3-methoxyphenyl)-5-methylfuran-2-carboxylic acid (compound XI-4-3) as the starting material to obtain a light yellow solid, yield was 30%; ESA-MS: m/z=364.0 [M+H]⁺.

### N-(3-(chloromethyl)-1,2,4-thiadiazol-5-yl)-5-methyl-4-(3-(trifluoromethoxy)phenyl)furan-2-carboxamide (Compound F-22)

Preparation of compound F-22 referred to the synthesis of compound A-105, using 5-methyl-4-(3-(trifluoromethoxy)phenyl)furan-2-carboxylic acid (compound XI-4-4) as the starting material to obtain a light yellow solid, yield was 32%; ESA-MS: m/z=418.0 [M+H]⁺.

### N-(3-(chloromethyl)-1,2,4-thiadiazol-5-yl)-4-(3-(difluoromethoxy)phenyl)-5-methylfuran-2-carboxamide (Compound F-23)

Preparation of compound F-23 referred to the synthesis of compound A-105, using 4-(3-(difluoromethoxy)phenyl)-5-methylfuran-2-carboxylic acid (compound XI-4-5) as the starting material to obtain a light yellow solid, yield was 27%; ESA-MS: m/z=400.0 [M+H]⁺.

### 5-Methyl-N-(3-(pyrrolidin-1-ylmethyl)-1,2,4-thiadiazol-5-yl)-4-(3-(trifluoromethyl)phenyl)furan-2-carboxamide (Compound F-24)

Preparation of compound F-24 referred to the synthesis of compound A-106, using compound F-19 and pyrrolidine as the starting material to obtain a light yellow solid, yield was 72%; ESA-MS: m/z=437.1 [M+H]⁺.

### 4-(3-Cyanophenyl)-5-methyl-N-(3-(piperidin-1-ylmethyl)-1,2,4-thiadiazol-5-yl)furan-2-carboxamide (Compound F-25)

Preparation of compound F-25 referred to the synthesis of compound A-106, using compound F-20 and piperidine as the starting material to obtain a light yellow solid, yield was 70%; ESA-MS: m/z=408.1 [M+H]⁺.

### 4-(3-Methoxyphenyl)-5-methyl-N-(3-(morpholinomethyl)-1,2,4-thiadiazol-5-yl)furan-2-carboxamide (Compound F-26)

Preparation of compound F-26 referred to the synthesis of compound A-106, using compound F-21 and morpholine as the starting material to obtain a light yellow solid, yield was 68%; ESA-MS: m/z=415.1 [M+H]⁺.

### 5-Methyl-N-(3-(morpholinomethyl)-1,2,4-thiadiazol-5-yl)-4-(3-(trifluoromethoxy)phenyl)furan-2-carboxamide (Compound F-27)

Preparation of compound F-27 referred to the synthesis of compound A-106, using compound F-22 and morpholine as the starting material to obtain a light yellow solid, yield was 66%; ESA-MS: m/z=469.1 [M+H]⁺.

### 4-(3-(Difluoromethoxy)phenyl)-5-methyl-N-(3-((4-methylpiperazin-1-yl)methyl)-1,2,4-thiadiazol-5-yl)furan-2-carboxamide (Compound F-28)

Preparation of compound F-28 referred to the synthesis of compound A-106, using compound F-23 and 1-methylpiperazine as the starting material to obtain a light yellow solid, yield was 71%; ESA-MS: m/z=464.1 [M+H]⁺.

### Example 54: Synthesis of Compounds G-1˜G-5

The synthesis route is shown in FIG. 62.

### N-(3-(2-oxopropyl)-1,2,4-thiadiazol-5-yl)-4-(3-(trifluoromethyl)phenyl)furan-2-carboxamide (Compound G-1)

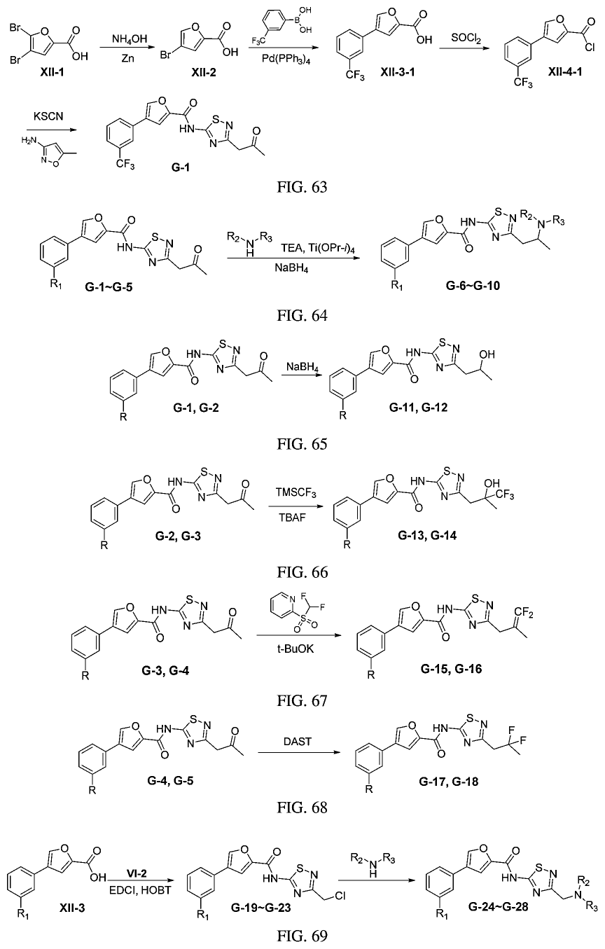

The specific synthesis route is shown in FIG. 63.

### (a) Synthesis of 4-bromofuran-2-carboxylic acid (Compound XII-2)

4,5-dibromofuran-2-carboxylic acid (XII-1, 1.00 g, 3.7 mmol), ammonium hydroxide (4 mL), and zinc powder (236 mg, 3.7 mmol) was added to 14 mL water in sequence, and then the reaction mixture was stirred at room temperature for 5 hours. After filtered through diatomite, the reaction mixture was acidified with 2 mol/L HCl. Ethyl acetate was added to the reaction mixture and the organic layer was washed with water and brine, dried over anhydrous Na₂SO₄, and concentrated to give a residue, which was purified by column-chromatograph to give a light yellow solid, yield was 86%; ESA-MS: m/z=191.0 [M+H]⁺.

### (b) Synthesis of 4-(3-(trifluoromethyl)phenyl)furan-2-carboxylic acid (Compound XII-3-1)

To a suspension of compound XII-2 (600 mg, 3.2 mmol) in isopropyl alcohol and water (3:1, 16 mL) was added 3-(trifluoromethyl)phenylboronic acid (630 mg, 3.3 mmol), sodium carbonate (620 mg, 5.8 mmol), bis(triphenylphosphine)palladium(II) chloride (65 mg, 0.09 mmol) under nitrogen atmosphere, and the reaction mixture was heated to reflux for 4 hours. The reaction system was then filtered to remove the solid, concentrated under reduced pressure and acidified with 6 mol/L HCl. Ethyl acetate was added to the reaction mixture and the organic layer was washed with water and brine, dried over anhydrous Na₂SO₄, and concentrated to give a residue, which was purified by column-chromatograph to give a light yellow solid, yield was 60%; ESA-MS: m/z=257.0 [M+H]⁺.

### (c) Synthesis of 4-(3-(trifluoromethyl)phenyl)furan-2-carbonyl chloride (Compound XII-4-1)

A solution of compound XII-3-1 (492 mg, 1.9 mmol) in 10 mL SOCl₂ was heated to reflux for 2.5 hours, and the mixture was concentrated to dryness to get a light yellow solid, yield was 95%;

(d) Synthesis of N-(3-(2-oxopropyl)-1,2,4-thiadiazol-5-yl)-4-(3-(trifluoromethyl)phenyl)furan-2-carboxamide (Compound G-1)

A suspension of compound XII-4-1 (494 mg, 1.8 mmol) and potassium thiocyanate (192 mg, 2.0 mmol) in 8 mL acetonitrile was stirred at 60° C. for 30 min, and then 5-methylisoxazol-3-amine (177 mg, 1.8 mmol) was added and stirred for another 1 hour at the same temperature. Ethyl acetate was added to the reaction mixture and the organic layer was washed with water and brine, dried over anhydrous Na₂SO₄, and concentrated to give a residue, which was purified by column-chromatograph to give a light yellow solid, yield was 83%; ¹H NMR (500 MHZ, Chloroform-d): δ 7.81 (d, J=1.5 Hz, 1H), 7.75 (t, J=1.5 Hz, 1H), 7.74-7.70 (m, 1H), 7.64 (t. J=7.5 Hz, 1H), 7.48-7.44 (m, 1H), 7.37 (d, J=1.5 Hz, 1H), 3.57 (s, 2H), 2.30 (s, 3H); ESA-MS: m/z=396.1 [M+H]⁺.

### 4-(3-Cyanophenyl)-N-(3-(2-oxopropyl)-1,2,4-thiadiazol-5-yl)furan-2-carboxamide (Compound G-2)

Preparation of compound G-2 referred to the synthesis of compound G-1, replacing (3-(trifluoromethyl)phenyl) boronic acid with (3-cyanophenyl) boronic acid to obtain a light yellow solid, yield was 39%; ESA-MS: m/z=353.1 [M+H]⁺.

### 4-(3-Methoxyphenyl)-N-(3-(2-oxopropyl)-1,2,4-thiadiazol-5-yl)furan-2-carboxamide (Compound G-3)

Preparation of compound G-3 referred to the synthesis of compound G-1, replacing (3-(trifluoromethyl)phenyl) boronic acid with (3-methoxyphenyl) boronic acid to obtain a light yellow solid, yield was 40%; ESA-MS: m/z=358.1 [M+H]⁺.

### N-(3-(2-oxopropyl)-1,2,4-thiadiazol-5-yl)-4-(3-(trifluoromethoxy)phenyl)furan-2-carboxamide (Compound G-4)

Preparation of compound G-4 referred to the synthesis of compound G-1, replacing (3-(trifluoromethyl)phenyl) boronic acid with (3-(trifluoromethoxy)phenyl) boronic acid to obtain a light yellow solid, yield was 39%; ESA-MS: m/z=412.0 [M+H]⁺.

### 4-(3-(Difluoromethoxy)phenyl)-N-(3-(2-oxopropyl)-1,2,4-thiadiazol-5-yl)furan-2-carboxamide (Compound G-5)

Preparation of compound G-5 referred to the synthesis of compound G-1, replacing (3-(trifluoromethyl)phenyl) boronic acid with (3-(difluoromethoxy)phenyl) boronic acid to obtain a light yellow solid, yield was 42%; ESA-MS: m/z=394.1 [M+H]⁺.

### Example 55: Synthesis of Compounds G-6˜G-10

The synthesis route is shown in FIG. 64.

### N-(3-(2-(pyrrolidin-1-yl)propyl)-1,2,4-thiadiazol-5-yl)-4-(3-(trifluoromethyl)phenyl) furan-2-carboxamide (Compound G-6)

Preparation of compound G-6 referred to the synthesis of compound A-38, using compound G-1 and pyrrolidine as the starting material to obtain a light yellow solid, yield was 60%; ESA-MS: m/z=451.1 [M+H]⁺.

### 4-(3-Cyanophenyl)-N-(3-(2-(piperidin-1-yl)propyl)-1,2,4-thiadiazol-5-yl)furan-2-carboxamide (Compound G-7)

Preparation of compound G-7 referred to the synthesis of compound A-38, using compound G-2 and piperidine as the starting material to obtain a light yellow solid, yield was 71%; ESA-MS: m/z=422.2 [M+H]⁺.

### 4-(3-Methoxyphenyl)-N-(3-(2-morpholinopropyl)-1,2,4-thiadiazol-5-yl)furan-2-carboxamide (Compound G-8)

Preparation of compound G-8 referred to the synthesis of compound A-38, using compound G-3 and morpholine as the starting material to obtain a light yellow solid, yield was 66%; ESA-MS: m/z=429.2 [M+H]⁺.

### N-(3-(2-(4-methylpiperazin-1-yl)propyl)-1,2,4-thiadiazol-5-yl)-4-(3-(trifluoromethoxy)phenyl)furan-2-carboxamide (Compound G-9)

Preparation of compound G-9 referred to the synthesis of compound A-38, using compound G-4 and 1-methylpiperazine as the starting material to obtain a light yellow solid, yield was 63%; ESA-MS: m/z=496.2 [M+H]⁺.

### 4-(3-(Difluoromethoxy)phenyl)-N-(3-(2-(4-methylpiperazin-1-yl)propyl)-1,2,4-thiadiazol-5-yl)furan-2-carboxamide (Compound G-10)

Preparation of compound G-10 referred to the synthesis of compound A-38, using compound G-5 and 1-methylpiperazine as the starting material to obtain a light yellow solid, yield was 67%; ESA-MS: m/z=478.2 [M+H]⁺.

### Example 56: Synthesis of Compounds G-11˜G-12

The synthesis route is shown in FIG. 65.

### N-(3-(2-hydroxypropyl)-1,2,4-thiadiazol-5-yl)-4-(3-(trifluoromethyl)phenyl)furan-2-carboxamide (Compound G-11)

Preparation of compound G-11 referred to the synthesis of compound A-60, using compound G-1 as the starting material to obtain a light yellow solid, yield was 83%; ESA-MS: m/z=398.1 [M+H]⁺.

### 4-(3-Cyanophenyl)-N-(3-(2-hydroxypropyl)-1,2,4-thiadiazol-5-yl)furan-2-carboxamide (Compound G-12)

Preparation of compound G-12 referred to the synthesis of compound A-60, using compound G-2 as the starting material to obtain a light yellow solid, yield was 81%; ESA-MS: m/z=355.1 [M+H]⁺.

### Example 57: Synthesis of Compounds G-13˜G-14

The synthesis route is shown in FIG. 66.

### 4-(3-Cyanophenyl)-N-(3-(3,3,3-trifluoro-2-hydroxy-2-methylpropyl)-1,2,4-thiadiazol-5-yl) furan-2-carboxamide (Compound G-13)

Preparation of compound G-13 referred to the synthesis of compound A-75, using compound G-2 as the starting material to obtain a light yellow solid, yield was 73%; ESA-MS: m/z=423.1 [M+H]⁺.

### 4-(3-Methoxyphenyl)-N-(3-(3,3,3-trifluoro-2-hydroxy-2-methylpropyl)-1,2,4-thiadiazol-5-yl)furan-2-carboxamide (Compound G-14)

Preparation of compound G-14 referred to the synthesis of compound A-75, using compound G-3 as the starting material to obtain a light yellow solid, yield was 68%; ESA-MS: m/z=428.1 [M+H]⁺.

### Example 58: Synthesis of Compounds G-15˜G-16

The synthesis route is shown in FIG. 67.

### N-(3-(3,3-difluoro-2-methylallyl)-1,2,4-thiadiazol-5-yl)-4-(3-methoxyphenyl)furan-2-carboxamide (Compound G-15)

Preparation of compound G-15 referred to the synthesis of compound A-80, using compound G-3 as the starting material to obtain a light yellow solid, yield was 68%; ESA-MS: m/z=392.1 [M+H]⁺.

### N-(3-(3,3-difluoro-2-methylallyl)-1,2,4-thiadiazol-5-yl)-4-(3-(trifluoromethoxy)phenyl) furan-2-carboxamide (Compound G-16)

Preparation of compound G-16 referred to the synthesis of compound A-80, using compound G-4 as the starting material to obtain a light yellow solid, yield was 61%; ESA-MS: m/z=446.1 [M+H]⁺.

### Example 59: Synthesis of Compounds G-17˜G-18

The synthesis route is shown in FIG. 68.

### N-(3-(2,2-difluoropropyl)-1,2,4-thiadiazol-5-yl)-4-(3-(trifluoromethoxy)phenyl)furan-2-carboxamide (Compound G-17)

Preparation of compound G-17 referred to the synthesis of compound A-85, using compound G-4 as the starting material to obtain a light yellow solid, yield was 63%; ESA-MS: m/z=434.1 [M+H]⁺.

### 4-(3-(Difluoromethoxy)phenyl)-N-(3-(2,2-difluoropropyl)-1,2,4-thiadiazol-5-yl)furan-2-carboxamide (Compound G-18)

Preparation of compound G-18 referred to the synthesis of compound A-85, using compound G-5 as the starting material to obtain a light yellow solid, yield was 60%; ESA-MS: m/z=416.1 [M+H]⁺.

### Example 60: Synthesis of Compounds G-19˜G-28

The synthesis route is shown in FIG. 69.

### N-(3-(chloromethyl)-1,2,4-thiadiazol-5-yl)-4-(3-(trifluoromethyl)phenyl)furan-2-carboxamide (Compound G-19)

Preparation of compound G-19 referred to the synthesis of compound A-105, using 4-(3-(trifluoromethyl)phenyl)furan-2-carboxylic acid (compound XII-3-1) as the starting material to obtain a light yellow solid, yield was 35%; ESA-MS: m/z=388.0 [M+H]⁺.

### N-(3-(chloromethyl)-1,2,4-thiadiazol-5-yl)-4-(3-cyanophenyl)furan-2-carboxamide (Compound G-20)

Preparation of compound G-20 referred to the synthesis of compound A-105, using 4-(3-cyanophenyl)furan-2-carboxylic acid (compound XII-3-2) as the starting material to obtain a light yellow solid, yield was 29%; ESA-MS: m/z=345.0 [M+H]⁺.

### N-(3-(chloromethyl)-1,2,4-thiadiazol-5-yl)-4-(3-methoxyphenyl)furan-2-carboxamide (Compound G-21)

Preparation of compound G-21 referred to the synthesis of compound A-105, using 4-(3-methoxyphenyl)furan-2-carboxylic acid (compound XII-3-3) as the starting material to obtain a light yellow solid, yield was 33%; ESA-MS: m/z=350.0 [M+H]⁺.

### N-(3-(chloromethyl)-1,2,4-thiadiazol-5-yl)-4-(3-(trifluoromethoxy)phenyl)furan-2-carboxamide (Compound G-22)

Preparation of compound G-22 referred to the synthesis of compound A-105, using 4-(3-(trifluoromethoxy)phenyl)furan-2-carboxylic acid (compound XII-3-4) as the starting material to obtain a light yellow solid, yield was 32%; ESA-MS: m/z=404.0 [M+H]⁺.

### N-(3-(chloromethyl)-1,2,4-thiadiazol-5-yl)-4-(3-(difluoromethoxy)phenyl)furan-2-carboxamide (Compound G-23)

Preparation of compound G-23 referred to the synthesis of compound A-105, using 4-(3-(difluoromethoxy)phenyl)furan-2-carboxylic acid (compound XII-3-5) as the starting material to obtain a light yellow solid, yield was 28%; ESA-MS: m/z=386.0 [M+H]⁺.

### N-(3-(pyrrolidin-1-ylmethyl)-1,2,4-thiadiazol-5-yl)-4-(3-(trifluoromethyl)phenyl)furan-2-carboxamide (Compound G-24)

Preparation of compound G-24 referred to the synthesis of compound A-106, using compound G-19 and pyrrolidine as the starting material to obtain a light yellow solid, yield was 70%; ESA-MS: m/z=423.1 [M+H]⁺.

### 4-(3-Cyanophenyl)-N-(3-(piperidin-1-ylmethyl)-1,2,4-thiadiazol-5-yl)furan-2-carboxamide (Compound G-25)

Preparation of compound G-25 referred to the synthesis of compound A-106, using compound G-20 and piperidine as the starting material to obtain a light yellow solid, yield was 60%; ESA-MS: m/z=394.1 [M+H]⁺.

### 4-(3-Methoxyphenyl)-N-(3-(morpholinomethyl)-1,2,4-thiadiazol-5-yl)furan-2-carboxamide (Compound G-26)

Preparation of compound G-26 referred to the synthesis of compound A-106, using compound G-21 and morpholine as the starting material to obtain a light yellow solid, yield was 62%; ESA-MS: m/z=401.1 [M+H]⁺.

### N-(3-(morpholinomethyl)-1,2,4-thiadiazol-5-yl)-4-(3-(trifluoromethoxy)phenyl)furan-2-carboxamide (Compound G-27)

Preparation of compound G-27 referred to the synthesis of compound A-106, using compound G-22 and 1-methylpiperazine as the starting material to obtain a light yellow solid, yield was 63%; ESA-MS: m/z=468.1 [M+H]⁺.

### 4-(3-(Difluoromethoxy)phenyl)-N-(3-((4-methylpiperazin-1-yl)methyl)-1,2,4-thiadiazol-5-yl)furan-2-carboxamide (Compound G-28)

Preparation of compound G-28 referred to the synthesis of compound A-106, using compound G-23 and 1-methylpiperazine as the starting material to obtain a light yellow solid, yield was 73%; ESA-MS: m/z=450.1 [M+H]⁺0.23

### Biological Test Example 1: Evaluation of AR Transcriptional Activity

Detection principles: We conducted a stable transfection of promoter ARR2PB known for its strong AR responsiveness, into the androgen-dependent PCa cells (LNCaP). The expression of enhanced green fluorescent protein (eGFP) is depending on the degree of activation of AR. Therefore, LNCaP-ARR2PB-eGFP cell line could quantitatively determine the AR antagonistic activities of the compounds by evaluating the expression of eGFP.

Methods: The cells of LNCaP-ARR2PB-eGFP were cultured in RPMI-1640 media supplemented with fetal bovine serum to 80% confluency. Then, the cells were starved with RPMI-1640 media supplemented with 5% charcoal stripped serum (CSS) for 5 days, followed by seeding into a 96 well plate (3.5×10⁴ cells/well). After 1 day incubation, the cells were treated with 5 nM dihydrotestosterone (DHT) and intended concentrations of compounds (examples 1-60). After 72 hours incubation, the fluorescence intensities were evaluated using microplate reader (excitation, 485 nm; emission, 535 nm) and the results were converted to half maximal inhibitory concentration (IC₅₀) of AR antagonistic activities.

Results: As shown in Table 2, 190 of the 403 synthesized compounds in examples 1-60 shows considerable AR antagonistic activity at AR transcription level (<50 μM). The activities of some compounds exhibit similar AR antagonistic activity compared with Enzalutamide, such as compounds A-1, A-2, A-14, A-34, A-37, etc.

TABLE 2
AR transcriptional activities of synthesized compounds

| No. | IC₅₀(μM) |
|---|---|
| A-1 | 0.03 |
| A-2 | 0.04 |
| A-3 | 0.32 |
| A-4 | 0.35 |
| A-5 | 0.95 |
| A-6 | 0.13 |
| A-7 | 1.16 |
| A-8 | 1.71 |
| A-9 | 2.36 |
| A-10 | 1.24 |
| A-11 | 4.24 |
| A-12 | 3.17 |
| A-13 | 0.48 |
| A-14 | 0.05 |
| A-24 | 1.62 |
| A-25 | 13.59 |
| A-26 | 3.21 |
| A-27 | 5.50 |
| A-28 | 1.03 |
| A-29 | 0.94 |
| A-30 | 5.19 |
| A-31 | 6.33 |
| A-34 | 0.04 |
| A-35 | 0.72 |
| A-36 | 0.07 |
| A-37 | 0.03 |
| A-38 | 0.09 |
| A-39 | 0.12 |
| A-40 | 0.23 |
| A-41 | 0.17 |
| A-42 | 0.36 |
| A-43 | 0.04 |
| A-44 | 0.06 |
| A-45 | 0.04 |
| A-46 | 0.41 |
| A-49 | 0.21 |
| A-52 | 0.76 |
| A-56 | 0.84 |
| A-60 | 0.39 |
| A-61 | 3.82 |
| A-66 | 1.39 |
| A-69 | 0.54 |
| A-70 | 0.88 |
| A-75 | 0.09 |
| A-80 | 0.81 |
| A-85 | 0.77 |
| A-90 | 0.05 |
| A-93 | 0.03 |
| A-96 | 0.16 |
| A-99 | 10.39 |
| A-100 | 15.41 |
| A-105 | 22.95 |
| A-106 | 1.67 |
| A-107 | 1.77 |
| A-108 | 0.84 |
| A-109 | 0.29 |
| A-110 | 0.25 |
| A-111 | 0.57 |
| A-112 | 1.87 |
| A-118 | 2.23 |
| A-123 | 0.59 |
| A-124 | 0.04 |
| A-129 | 0.06 |
| A-130 | 0.67 |
| A-131 | 1.42 |
| B-1 | 18.58 |
| B-2 | 54.93 |
| B-3 | 12.57 |
| B-4 | 32.47 |
| B-5 | 19.56 |
| B-6 | 11.81 |
| B-7 | 15.02 |
| B-12 | 23.27 |
| B-14 | 33.58 |
| B-16 | 35.71 |
| B-19 | 59.30 |
| B-20 | 11.47 |
| B-25 | 15.48 |
| B-30 | 4.93 |
| B-35 | 14.51 |
| B-40 | 12.29 |
| B-47 | 18.57 |
| B-50 | 22.85 |
| B-52 | 25.14 |
| B-56 | 28.29 |
| B-57 | 23.53 |
| B-58 | 32.61 |
| C-1 | 9.33 |
| C-2 | 33.29 |
| C-3 | 43.72 |
| C-4 | 25.11 |
| C-5 | 49.29 |
| C-6 | 33.24 |
| C-7 | 10.55 |
| C-12 | 5.77 |
| C-14 | 19.36 |
| C-17 | 6.17 |
| C-19 | 15.43 |
| C-20 | 4.52 |
| C-25 | 2.51 |
| C-30 | 12.74 |
| C-35 | 29.10 |
| C-40 | 12.25 |
| C-47 | 10.35 |
| C-50 | 12.49 |
| C-53 | 14.79 |
| C-56 | 12.94 |
| C-57 | 13.28 |
| C-58 | 9.79 |
| D-1 | 24.97 |
| D-2 | 32.59 |
| D-3 | 13.64 |
| D-4 | 41.53 |
| D-5 | 39.98 |
| D-6 | 43.85 |
| D-7 | 23.17 |
| D-12 | 12.96 |
| D-14 | 37.18 |
| D-16 | 24.16 |
| D-19 | 19.24 |
| D-20 | 18.09 |
| D-25 | 32.96 |
| D-30 | 34.79 |
| D-35 | 49.54 |
| D-40 | 21.49 |
| D-47 | 25.34 |
| D-50 | 14.98 |
| D-53 | 27.32 |
| D-56 | 42.13 |
| D-57 | 15.01 |
| D-58 | 23.73 |
| E-1 | 1.06 |
| E-2 | 2.23 |
| E-3 | 0.57 |
| E-4 | 0.55 |
| E-5 | 1.48 |
| E-6 | 3.38 |
| E-8 | 7.54 |
| E-9 | 5.29 |
| E-10 | 0.99 |
| E-11 | 0.97 |
| E-12 | 1.57 |
| E-17 | 0.79 |
| E-22 | 1.46 |
| E-27 | 0.95 |
| E-32 | 1.32 |
| E-38 | 1.45 |
| E-39 | 2.19 |
| E-40 | 2.92 |
| E-41 | 0.89 |
| F-1 | 28.11 |
| F-2 | 34.34 |
| F-3 | 22.41 |
| F-4 | 35.72 |
| F-5 | 12.55 |
| F-6 | 17.89 |
| F-7 | 14.02 |
| F-8 | 17.61 |
| F-9 | 23.51 |
| F-10 | 27.07 |
| F-11 | 22.33 |
| F-13 | 31.54 |
| F-15 | 35.88 |
| F-17 | 24.95 |
| F-19 | 19.11 |
| F-24 | 35.21 |
| F-25 | 15.47 |
| F-26 | 31.87 |
| F-27 | 15.59 |
| F-28 | 16.44 |
| G-1 | 44.77 |
| G-2 | 35.52 |
| G-3 | 33.60 |
| G-4 | 11.58 |
| G-5 | 32.74 |
| G-6 | 13.86 |
| G-7 | 23.17 |
| G-8 | 19.22 |
| G-9 | 47.15 |
| G-10 | 15.32 |
| G-11 | 29.29 |
| G-13 | 22.01 |
| G-15 | 24.12 |
| G-17 | 22.99 |
| G-19 | 31.42 |
| G-24 | 24.15 |
| G-25 | 19.82 |
| G-26 | 27.13 |
| G-27 | 26.19 |
| G-28 | 39.20 |
| Enzalutamide | 0.02 |

### Biological Test Example 2: AR Competitive Ligand Binding Assay Showed that Compound A-1 does not Target the Conventional LBP of AR-LBD

Detection principles: The binding affinities of compounds to the LBP of AR-LBD were evaluated using PolarScreen™ Androgen Receptor Competitor Assay Kit. The AR-LBD (His-GST) can form a binary complex with a fluorescence ligand (FluormoneAL Green), and shows relatively high fluorescence polarization values. The addition of a test compound to a microporous plate containing the binary complex would reduce fluorescence polarization values if the compound could target LBP of AR-LBD by competing the fluorescence ligand. Otherwise, the fluorescence polarization values would remain unchanged if the test compound does not target to the AR LBP.

Methods: Various concentrations of compound A-1 were added to a buffer containing AR-LBD (His-GST) and fluorescence ligand (FluormoneAL Green). The natural ligand DHT was used as the positive control. After 4 hours incubation in darkness at room temperature, the fluorescence polarization values were determined using a multifunctional microplate reader.

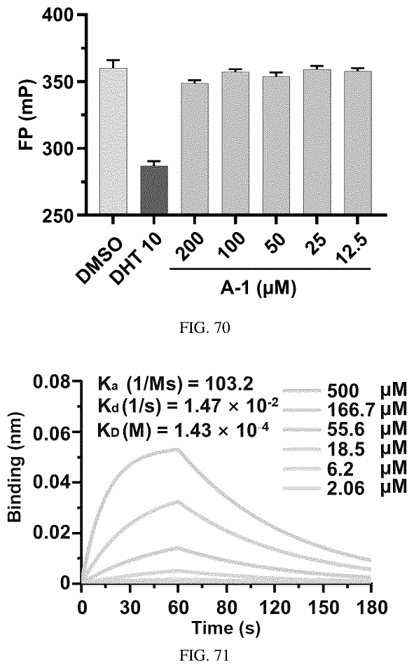

Results: As shown in FIG. 70, compound A-1 did not bind to the AR LBP within the concentrations ranging from 12.5 μM-200 μM, while DHT shows obvious binding to the AR LBP. Overall, A-1 does not target to LBP of AR-LBD.

### Biological Test Example 3: Detection of Direct Interaction Between A-1 and AR-LBD Using Biolayer Interferometry (BLI) Technique

Detection principles: The BLI technique can detect the interactions by monitoring the change of the interference spectrum in the sensor surface. BLI can quantitatively determine the binding and dissociation processes of a ligand to the target loaded sensor, by analyzing changes in the interference spectrum. Therefore, the direct interaction between ligand and target protein can be quantitatively detected.

Methods: The biotinylated AR-LBD protein was dissolved in the buffer of phosphate-buffered saline with 1% dimethyl sulfoxide. 200 μL of AR-LBD protein in the buffer was loaded onto sensors and equilibrated for 300 seconds. The kinetics of association between AR-LBD and A-1 were determined by soaking the AR-LBD loaded sensors into wells containing different concentrations of A-1 for 300 seconds, followed by soaking the sensors in the buffer for 180 seconds allow for dissociation. Finally, the ForteBio data analysis software was applied to calculate the kinetic binding constants (Kₐ, K_{d}, K_{D}).

Results: As shown in FIG. 71, compound A-1 (2.06-500 μM) forms direct interactions with the AR-LBD protein in a dose-dependent manner with a K_{D} of 1.43×10⁻⁴ M. The above results demonstrated A-1 targets the AR-LBD, but not AR LBP.

### Biological Test Example 4: Small-Angle X-Ray Scattering (SAXS) Technique to Detect AR-LBD Dimerization Disruption by A-1

Detection principles: SAXS is a vital technique for detecting conformational changes of biomacromolecule by quantitatively evalucate the overall dimension and shape silhouette of biomacromolecule.

Methods: The concentrations for AR-LBD, DHT, Enzalutamide and A-1 were 35 μM, 10 μM, 105 μM. 105 μM, respectively. The SAXS scattering signal were collected for 20 frames (1 second exposure time) using the BL19U2 beamline. The ATSAS software were applied to analysis the results.

Results: As shown in FIG. 72, compound A-1 inhibits AR-LBD dimerization, while AR-LBD bound with DHT/Enzalutamide maintains AR-LBD dimerization. These results demonstrate that unlike marketed drug Enzalutamide, compound A-1 is able to disrupt AR-LBD dimerization.

### Biological Test Example 5: Evaluation of the Antagonistic Activities of A-1 Toward Wild Type AR (AR^{WT}) and AR V684A (AR^{V684A}) Using Luciferase Reporter Assays

Detection principles: A reporter system for detecting the activities of firefly luciferase. To quantitatively detect the interaction between transcription factors and target promoters, the fluorescent intensities are detected to quantitatively evaluate the activities of firefly luciferase.

Methods: PC3 cells (no endogenous AR) treated with DMEM media supplemented with 5% CSS for 48 hours. Then, the PC3 cells (1.2×10⁴/well) were seeded into 96-well plates. After 16 hours, the PC3 cells were transfected with 20 ng/well plasmid and incubated for 20 min at room temperature. After 1 day incubation, the media in the wells was replaced, followed by the addition of various concentrations of compounds. After 48 hours, the culture medium was discarded and the cells were washed with 100 μL PBS, followed by addition of 30 μL 1×PLB and the 96-well plates was shaken on a shaker for 20 min to ensure complete lysis of the cells. Then, the cell lysis supernatant (20 μL/well) was transferred to a white opaque 96-well, and addition of 30 μL Luciferase Assay Reagent II to each well. Then, the fluorescent intensities of firefly luciferase were evaluated. Afterwards, 30 μL Stop&Glo Reagant was added to the wells, and the fluorescent intensities of firefly luciferase evaluated. Finally, the results were calculated according to the above data.

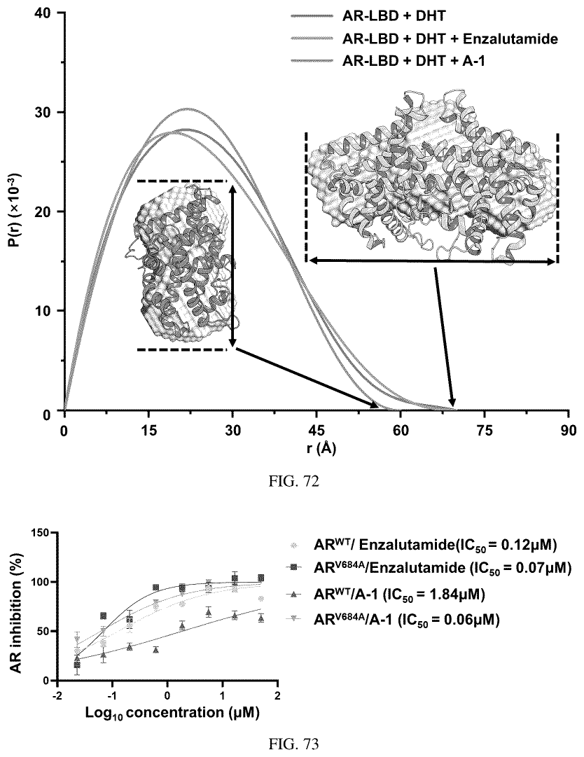

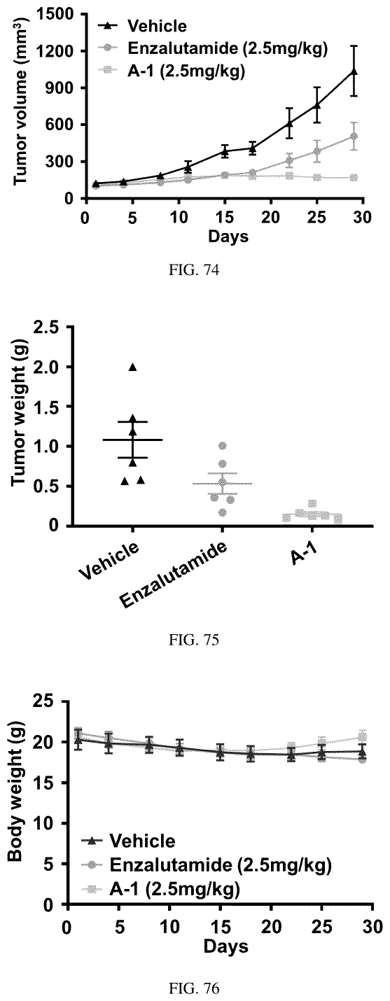

Results: As shown in FIG. 73, the dose-response curves for Enzalutamide toward full-length (FL)-AR^{WT} and FL-AR^{V684A} (IC₅₀=0.12 μM and 0.07 μM) are similar and the difference was only 1.7 times. In contrast, the dose-response curves for compound A-1 toward FL-AR^{WT} and FL-AR^{V684A} (IC₅₀=1.84 μM and 0.06 μM) exhibit significant variation, with a difference of 30.7 times. These results suggest that V684 (located in the AR-LBD dimer interface) is a key residue for the binding of compound A-1 to the AR. Overall, A-1 acts as an antagonist targeting the dimer interface of AR-LBD, as indicated by the results of biological test examples 2-5.

### Biological Test Example 6: The Efficacy of A-1 In Vivo Evaluated Using Xenograft Model

Methods: LNCaP cells were injected to the Male CB17 SCID mice (6-8 weeks old). Once tumor size reached target value, the mice were randomized into three groups with 6-8 mice per group. Direct intratumor injections of vehicle (saline), Enzalutamide (2.5 mg/kg/week), and A-1 (2.5 mg/kg/week) were administered to the established subcutaneous xenografts, individually. The mice weight and tumor volume were periodically recorded. Finally, the tumors were harvested.

Results: The tumor growth inhibition, including tumor volume (FIG. 74) and tumor weight (FIG. 75) for compound A-1 is much superior to that for Enzalutamide. Furthermore, no significant loss of body weight was observed for Enzalutamide and compound A-1 (FIG. 76). These results demonstrate A-1 possesses relatively high tumor inhibition capability and safety.

1. A thiadiazolamide compound of Formula (I), 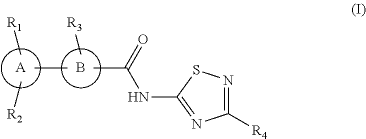
wherein:
ring A is selected from 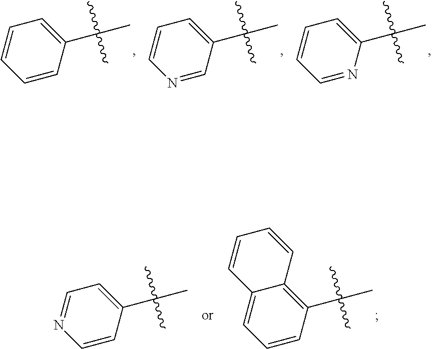 [SMILES: *c1ccccc1 ; *c1cccnc1 ; *c1ccccn1 ; *c1ccncc1 ; *c1cccc2ccccc12]
R₁ and R₂ are each independently selected from hydrogen, halogen, —NO₂, —CN, —OH, —NH₂, —SCH₃, carboxyl, ester, C₁₋₆ alkyl, C₁₋₆ fluoroalkyl, C₁₋₆ alkoxy, C₁₋₆ fluoroalkoxy, C₁₋₆ alkylamine group, C₁₋₆ dialkylamine group, C₃₋₆ cycloalkyl amine group, C₁₋₃ alkyl sulfonyl, C₁₋₃ fluoroalkyl sulfonyl, —NR₅R₆, CH₃ (CH₂), CONH— (n=1-6), —CONH₂, —CONR₅R₆, —SO₂NH₂, —SO₂NR₅R₆;
ring B is selected from 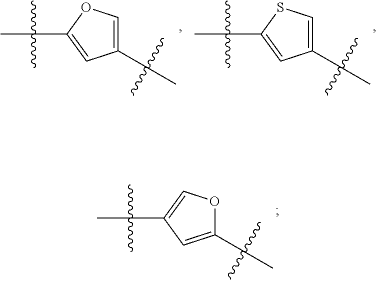 [SMILES: *c1coc(*)c1 ; *c1csc(*)c1 ; *c1coc(*)c1]
R₃ is selected from hydrogen, halogen, —NO₂, —CN, —OH, —NH₂, —SO₂NH₂, C₁₋₆ alkyl, C₁₋₆ fluoroalkyl, C₁₋₆ alkoxy, C₁₋₆ fluoroalkoxy;
R₄ is selected from hydrogen, halogen, C₁₋₆ alkyl, C₁₋₆ fluoroalkyl, C₁₋₆ alkoxy, C₁₋₆ fluoroalkoxy, C₁₋₆ alkylamine group, C₁₋₆ dialkylamine group, C₃₋₆ cycloalkyl amine group, CH₃ (CH₂), CONH— (n=1-6), —CONH₂, —CONR₅R₆, —CH₂COCH₃, —CH₂NR₅R₆, —CH₂CF₂CH₃, —CH₂C(═CF₂) CH₃, —CH₂C(═NHOH) CH₃, —CH₂C(═NHOCH₃)CH₃, —CH₂R₇, —CH₂C(R₇R₈)CH₃;
R₅ and R₆ are each independently selected from hydrogen, C₁₋₄ alkyl, C₁₋₄ cycloalkyl, or NR₅R₆ is 3-8 optionally substituted cyclic amine selected from morpholine, piperazine, methylpiperazine, pyrrolidine, piperidine, the substituent group is selected from hydrogen, C₁₋₆ alkyl, C₁₋₆ alkoxy, C₂₋₆ unsaturated aliphatic hydrocarbon group, C₃₋₈ cycloalkyl, C₃₋₈ unsaturated alicyclic group, C₃₋₈ saturated heterocyclic group, halogen, —NH₂, —CN;
R₇ and R₈ are each independently selected from hydrogen, fluoro, —OH, —NH₂, —CN, C₁₋₆ alkyl, C₁₋₆ fluoroalkyl, C₁₋₆ alkoxy, C₁₋₆ fluoroalkoxy, C₁₋₆ alkylamine group, C₁₋₆ dialkylamine group, optionally substituted C₃₋₈ cyclic amine selected from morpholine, piperazine, methylpiperazine, pyrrolidine, piperidine, the substituent group is selected from hydrogen, C₁₋₆ alkyl, C₁₋₆ alkoxy, C₂₋₆ unsaturated aliphatic hydrocarbon group, C₃₋₈ cycloalkyl, C₃₋₈ unsaturated alicyclic group, C₃₋₈ saturated heterocyclic group, halogen, —NH₂, —CN.

2. The thiadiazolamide compound according to claim 1, wherein R₁ and R₂ are are each independently selected from hydrogen, —F, —Cl, —NO₂, —CN, —CH₃, —OCH₃, —OCF₂H, —OCF₃, difluoroethoxy, trifluoroethoxy, CH₃SO₂—, CF₃SO₂—, —N(CH₃)₂, —N(CH₂CH₃)₂, CH₃CONH—, —CONH₂, —CONHCH₃, —SO₂NH₂, —SO₂NHCH₃.

3. The thiadiazolamide compound according to claim 1, wherein R³ is selected from hydrogen, —F, —Cl, —NO₂, —CH₃, —CH₂CH₃, —CF₃, —OCH₃, —OCF₂H, —OCF₃.

4. The thiadiazolamide compound according to claim 1, wherein R⁴ is selected from hydrogen, —F, —Cl, —CH₃, —N(CH₃)₂, —CONH₂, —CONH₂CH₂CH₃,

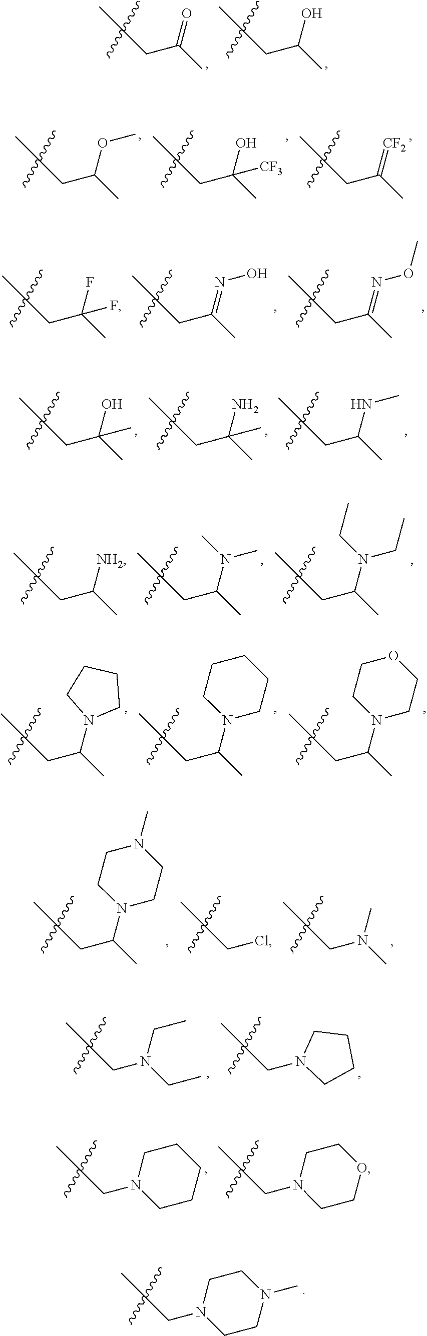

5. The thiadiazolamide compound according to claim 1, wherein the compound is

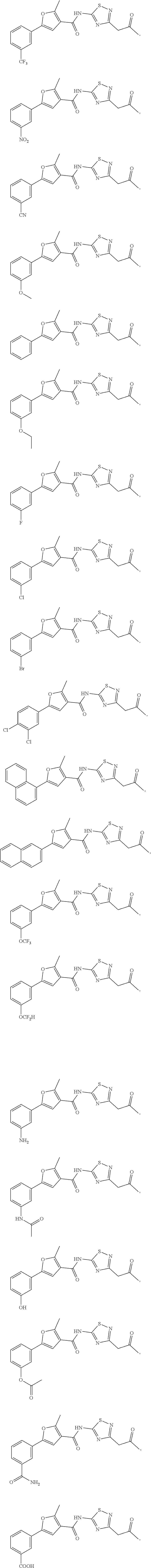

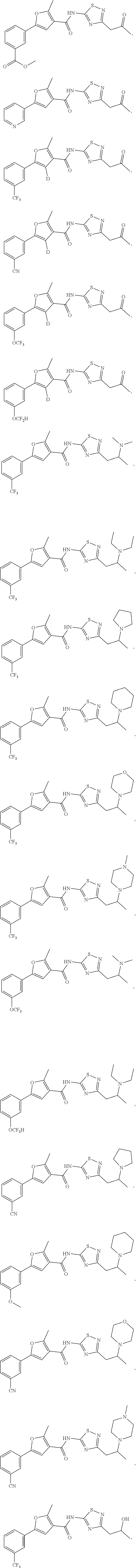

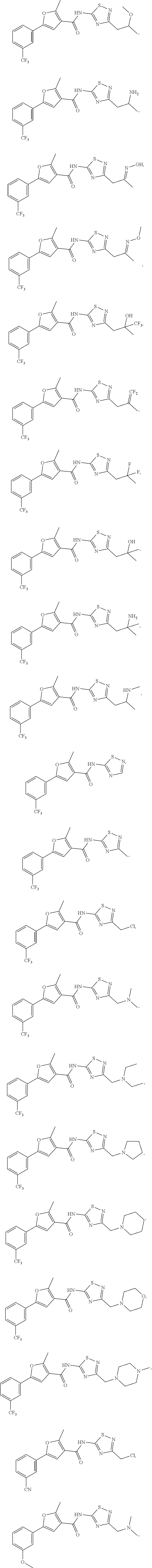

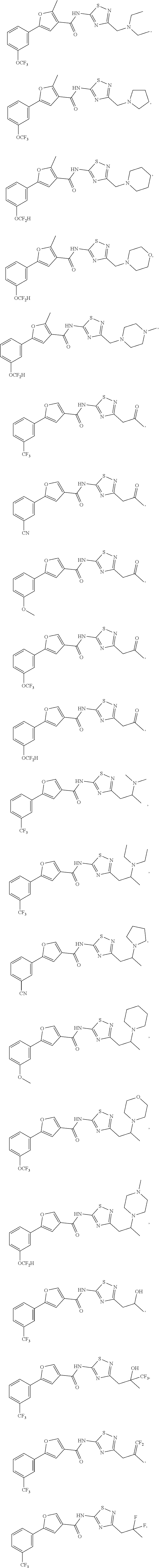

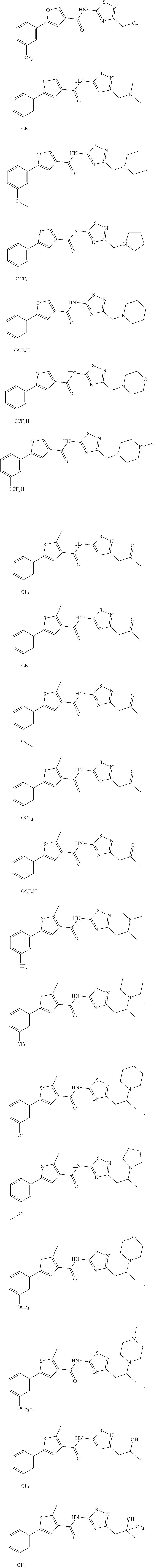

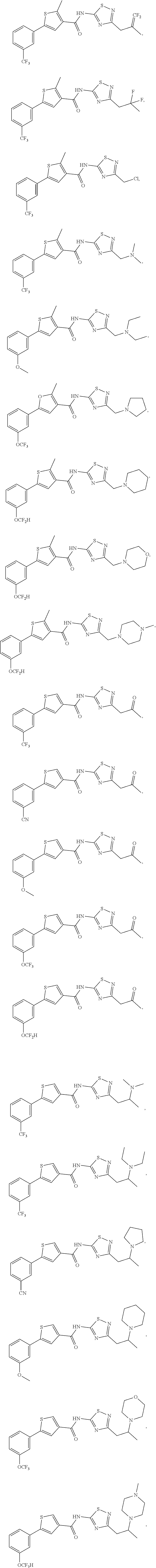

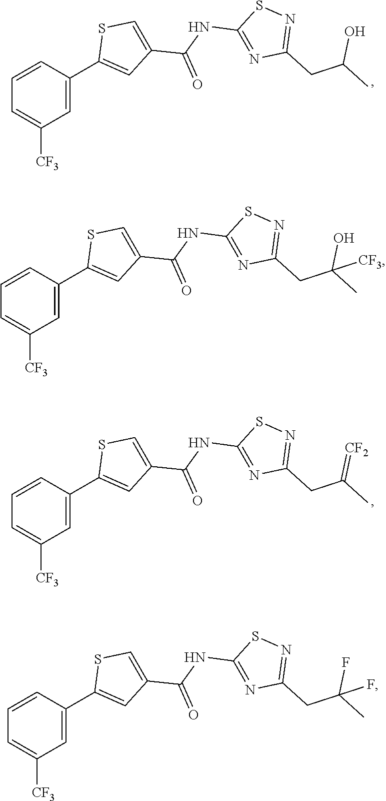

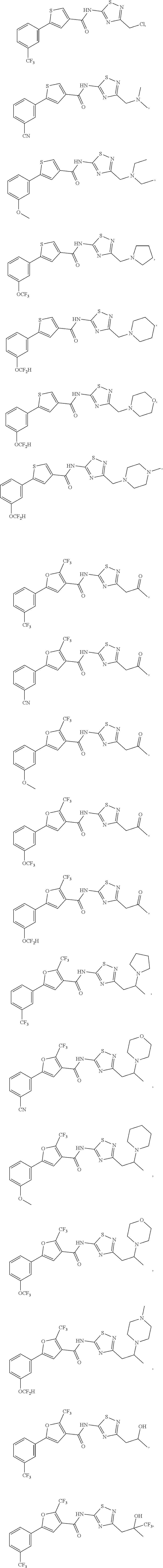

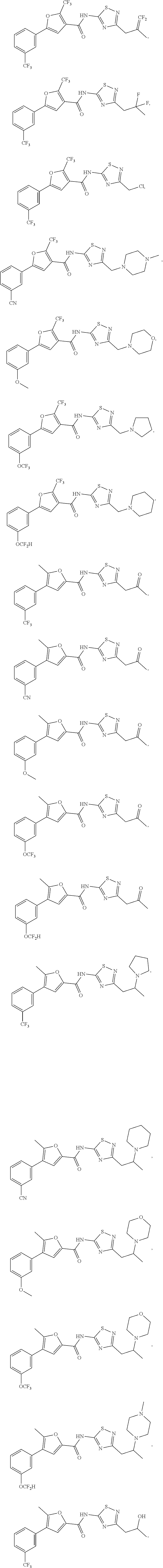

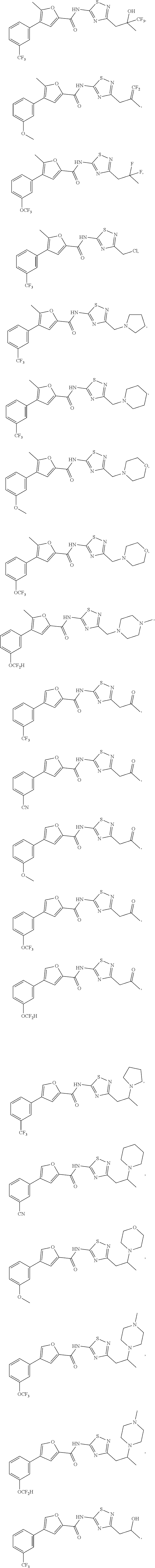

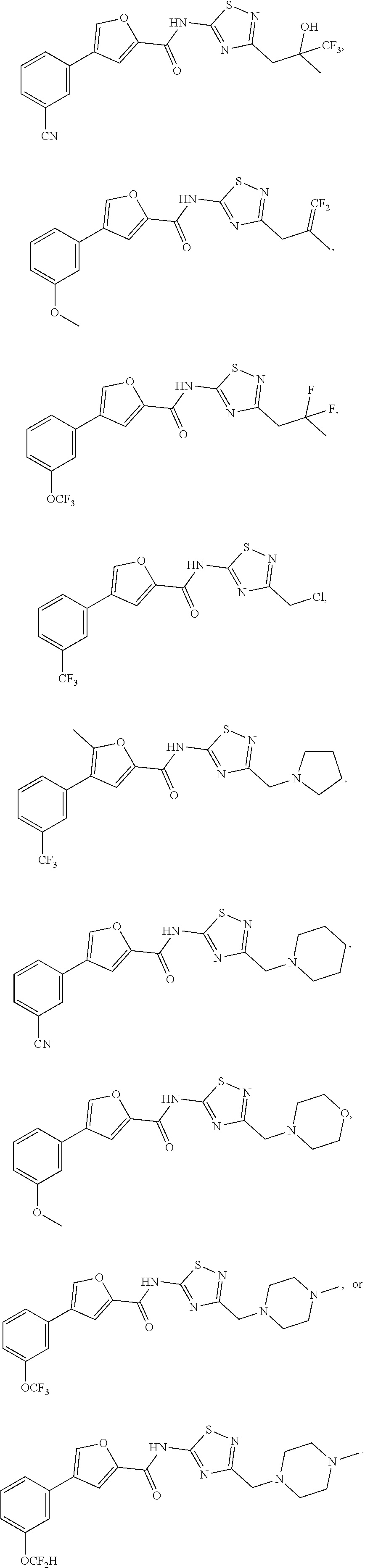

6. The thiadiazoleamide compounds according to claim 1, or their pharmaceutically acceptable salts, stereoisomers, prodrug molecules, deuterated products for use in the of preparation and treatment of prostate cancer, metastatic prostate cancer, castration-resistant prostate cancer, breast cancer, and ovarian cancer.

7. The applications as described in claim 6, the compounds inhibit tumor cell proliferation by antagonizing AR activity.

8. The applications as described in claim 7, the compounds are capable of disrupting AR-LBD dimerization, thereby possessing AR antagonstic activities.

9. A pharmaceutical composition comprising one or more thiadiazoleamide compounds as claimed in claim 1, or pharmaceutically acceptable salts, or stereoisomers, or prodrug molecules, or deuterated products, and pharmaceutically acceptable carriers.
